(19) United States
(12) Patent Application Publication
(10) Pub. No.: US 2021/0198567 A1
(43) Pub. Date: Jul. 1, 2021

(54) ORGANIC ELECTROLUMINESCENT COMPOUND, A PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Ji-Song Jun, Gyeonggi-do (KR); Young-Kwang Kim, Gyeonggi-do (KR); Kyoung-Jin Park, Gyeonggi-do (KR); So-Mi Park, Gyeonggi-do (KR)

(21) Appl. No.: 17/119,686

(22) Filed: Dec. 11, 2020

(30) Foreign Application Priority Data
Dec. 24, 2019 (KR) 10-2019-0173907

(51) Int. Cl. C09K 11/06; H01L 51/00

(52) U.S. Cl. CPC C09K 11/06; H01L 51/0072; C09K 2211/1022; H01L 51/0074; H01L 51/0073

(57) ABSTRACT

The present disclosure relates to an organic electroluminescent compound represented by formula 1, a plurality of host materials comprising a specific combination of compounds, and an organic electroluminescent device comprising the same. By comprising the organic electroluminescent compound or the specific combination of compounds according to the present disclosure as host materials, it is possible to provide an organic electroluminescent device with a longer lifetime property compared to conventional organic electroluminescent devices.

### TECHNICAL FIELD

The present disclosure relates to an organic electroluminescent compound, host materials comprising a combination of specific compounds, and an organic electroluminescent device comprising the same.

### BACKGROUND ART

An electroluminescent (EL) device is a self-light-emitting display device which has advantages in that it provides a wider viewing angle, a greater contrast ratio, and a faster response time. The first organic electroluminescent device was developed by Eastman Kodak in 1987, by using small aromatic diamine molecules and aluminum complexes as materials for forming a light-emitting layer (see Appl. Phys. Lett. 51, 913, 1987).

An organic electroluminescent device (OLED) changes electric energy into light by applying electricity to an organic electroluminescent material, and commonly comprises an anode, a cathode, and an organic layer formed between the two electrodes. The organic layer of the OLED may comprise a hole injection layer, a hole transport layer, a hole auxiliary layer, a light-emitting auxiliary layer, an electron blocking layer, a light-emitting layer, an electron buffer layer, a hole blocking layer, an electron transport layer, an electron injection layer, etc. The materials used in the organic layer can be classified into a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc., depending on their functions. In the OLED, holes from the anode and electrons from the cathode are injected into a light-emitting layer by the application of electric voltage, and excitons having high energy are produced by the recombination of the holes and electrons. The organic light-emitting compound moves into an excited state by the energy and emits light from energy when the organic light-emitting compound returns to the ground state.

The most important factor determining luminous efficiency in an OLED is light-emitting materials. A light-emitting material must have high quantum efficiency, and high electron and hole mobility, and the formed light-emitting material layer must be uniform and stable. Light-emitting materials may be categorized into blue, green, and red light-emitting materials dependent on the light-emitting color, and may further include yellow or orange light-emitting materials. In addition, light-emitting materials can also be categorized into host and dopant materials according to their functions. Recently, the development of an OLED providing high efficiency and long lifetime is an urgent issue. In particular, considering EL characteristic requirements for a middle or large-sized panel of OLED, materials showing better characteristics than conventional ones must be urgently developed. For this, as a solvent in a solid state and an energy transmitter, a host material should preferably have high purity and a molecular weight appropriate for vacuum deposition. Furthermore, a material is required to have high glass transition temperature and high pyrolysis temperature to achieve thermal stability, high electrochemical stability to achieve a long lifetime, ease of forming an amorphous thin film, good adhesion to the materials of adjacent layers, and non-migration to other layers.

At present, OLEDs primarily use phosphorescent materials having excellent luminous efficiency in panel implementation. In many applications such as TVs and lightings, the lifetime of OLED is insufficient and high efficiency of OLED is still required. Typically, the higher the luminance an OLED is, the shorter the lifetime of that same OLED. Therefore, a new light-emitting material having a long lifetime property is required for long-time use and high resolution of a display.

### DISCLOSURE OF INVENTION

### Technical Problem

The objective of the present disclosure is to provide an organic electroluminescent compound having a new structure appropriate to be applied to an organic electroluminescent device. Another objective of the present disclosure is to provide an improved host material capable of providing an organic electroluminescent device having a long lifetime property.

### Solution to Problem

The present inventors found that the above objective can be achieved by a compound represented by the following formula 1. The compound represented by the following formula 1 may be applied to an organic electroluminescent device as a plurality of host materials in combination with a compound represented by the following formula 2:

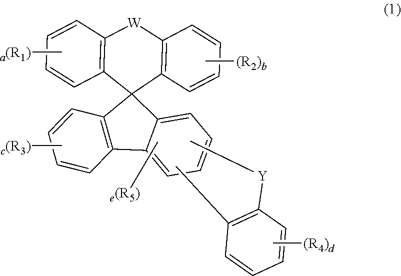

wherein

W represents a single bond, O, S, CR₆R₇, or N-L-R₈;

Y represents O, S, CR₆R₇, or N-L-R₈;

L, each independently, represents a single bond, a substituted or unsubstituted (C6-C30)arylene, or a substituted or unsubstituted (3- to 30-membered)heteroarylene;

R₁ to R₆, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arysilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or adjacent ones of R₁ to R₈ may be linked to each other to form a ring(s); and

a to d, each independently, represent an integer of 1 to 4; and e represents an integer of 1 or 2; where each of a to e is an integer of 2 or more, each of R₁, each of R₂, each of R₃, each of R₄, and each of R₅ may be the same or different;

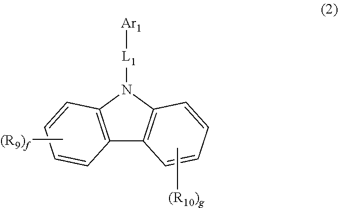

wherein

L₁ represents a single bond, or a substituted or unsubstituted (C6-C30)arylene;

Ar₁ represents a substituted or unsubstituted (C6-C30)aryl, or a substituted or unsubstituted nitrogen-containing (13- to 30-membered)heteroaryl;

R₉ and R₁₀, each independently, represent deuterium, a halogen, a cyano, a substituted or unsubstituted (C1-C30)alkyl, a substituted or unsubstituted (C6-C30)aryl, a substituted or unsubstituted (3- to 50-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or adjacent ones of R₉ and R₁₀ may be linked to each other to form a substituted or unsubstituted ring(s); with the proviso that if R₉ or R₁₀ represents a substituted (C6-C30)aryl, the substituent of the substituted (C6-C30)aryl is not a carbazolyl or a dibenzofuranyl, and if R₉ or R₁₀ forms a substituted ring, the substituent of the substituted ring does not comprise a triazinyl; and

f and g, each independently, represent an integer of 0 to 4, with the proviso that f and g are not zero at the same time; where each of f and g is an integer of 2 or more, each of R₉ and each of R₁₀ may be the same or different.

### Advantageous Effects of Invention

The organic electroluminescent compound according to the present disclosure exhibits performances appropriate to be used in an organic electroluminescent device. In addition, by comprising a specific combination of compounds according to the present disclosure as host materials, an organic electroluminescent device having longer lifetime property compared to conventional organic electroluminescent devices can be provided, and it is possible to manufacture a display system or a lighting system using the same.

### MODE FOR THE INVENTION

Hereinafter, the present disclosure will be described in detail. However, the following description is intended to explain the present disclosure, and is not meant in any way to restrict the scope of the present disclosure.

The term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, or an electron injection material, etc.

The term “a plurality of organic electroluminescent materials” in the present disclosure means an organic electroluminescent material(s) comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds which may be comprised in at least one of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. At least two compounds may be comprised in the same layer or different layers through methods used in the art, and may be mixture-evaporated or co-evaporated, or may be individually evaporated.

The term “a plurality of host materials” in the present disclosure means a host material(s) comprising a combination of at least two compounds, which may be comprised in any light-emitting layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of host materials of the present disclosure may be a combination of at least two host materials, and selectively may further comprise conventional materials comprised in an organic electroluminescent material. At least two compounds comprised in the plurality of host materials may be comprised together in one light-emitting layer or may respectively be comprised in different light-emitting layers. For example, the at least two host materials may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The alkyl may include methyl, ethyl, n-propyl, iso-propyl, n-butyl, iso-butyl, tert-butyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The cycloalkyl may include cyclopropyl, cyclobutyl, cyclopentyl, cyclohexyl, etc. The term “(3- to 7-membered)heterocycloalkyl” is meant to be a cycloalkyl having 3 to 7 ring backbone atoms, and including at least one heteroatom selected from the group consisting of B, N, O, S, Si, and P, and preferably the group consisting of O, S, and N. The heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms. The aryl may be partially saturated, and may comprise a spiro structure. The aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, etc. Specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, etc.

The term “(3- to 50-membered)heteroaryl(ene)” is an aryl or an arylene having 3 to 50 ring backbone atoms, preferably 3 to 30 ring backbone atoms, and including at least one, preferably 1 to 4 heteroatoms selected from the group consisting of B, N, O, S, Si, and P. The heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, naphthobenzofuranyl, naphthobenzothiophenyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazinyl, phenanthridinyl, benzodioxolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyl-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthrdinyl, 4-phenanthridinyl, 6-phenanthrdinyl, 7-phenanthrdinyl, 8-phenanthridinyl, 9-phenanthridinyl, 10-phenanthridinyl, 1-acridinyl, 2-acridinyl, 3-acidinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, etc. “Halogen” includes F, Cl, Br, and I.

In addition, “substituted” in the expression “substituted or unsubstituted” means that a hydrogen atom in a certain functional group is replaced with another atom or another functional group, i.e., a substituent. In the present disclosure, the substituents of the substituted alkyl, the substituted aryl(ene), the substituted heteroaryl(ene), the substituted trialkylsilyl, the substituted dialkylarylsilyl, the substituted alkyldiarylsilyl, the substituted triarylsilyl, the substituted mono- or di-alkylamino, the substituted alkylarylamino, the substituted mono- or di-arylamino, and the substituted ring(s), each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3-to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituents, each independently, are at least one selected from the group consisting of deuterium; a cyano; a (C1-C20)alkyl; a (5- to 25-membered)heteroaryl unsubstituted or substituted with a (C6-C25)aryl(s); an unsubstituted (C6-C25)aryl; and a tri(C6-C25)arylsilyl. According to another embodiment of the present disclosure, the substituents, each independently, are at least one selected from the group consisting of a cyano; a (C1-C10)alkyl; a (5- to 20-membered)heteroaryl unsubstituted or substituted with a (C6-C18)aryl(s); an unsubstituted (C6-C18)aryl; and a tri(C6-C18)arylsilyl. For example, the substituents, each independently, may be at least one selected from the group consisting of a cyano; a methyl; a phenyl; a naphthyl; a biphenyl; a naphthylphenyl; a terphenyl; a triphenylenyl; a triazinyl substituted with at least one of a phenyl(s), a naphthyl(s), and a biphenyl(s); a pyridyl substituted with a phenyl(s); a pyrimidinyl substituted with a phenyl(s); a dibenzofuranyl; a carbazolyl substituted with a phenyl(s); and a triphenylsilyl.

Herein, a ring formed by a linkage of adjacent substituents means that at least two adjacent substituents are linked to or fused with each other to form a substituted or unsubstituted mono- or polycyclic (3- to 30-membered) alicyclic or aromatic ring, or the combination thereof; preferably, a substituted or unsubstituted mono- or polycyclic (3- to 26-membered) alicyclic or aromatic ring, or the combination thereof; and more preferably, a mono- or polycyclic (5- to 25-membered) aromatic ring unsubstituted or substituted with at least one of a (C6-C18)aryl(s) and a (3- to 20-membered)heteroaryl(s). In addition, the formed ring may contain at least one heteroatom selected from B, N, O, S, Si, and P, preferably at least one heteroatom selected from N, O, and S. For example, the ring may be a benzene ring; an indole ring substituted with at least one of a phenyl(s), a biphenyl(s), a naphthyl(s), a naphthylphenyl(s), a phenylnaphthyl(s), a terphenyl(s), a triphenylenyl(s), a phenylpyridyl(s), and a phenylpyrimidinyl(s); a spiro[indene-xanthene] ring unsubstituted or substituted with a phenylcarbazolyl(s); a xanthene ring unsubstituted or substituted with a phenylcarbazolyl(s), etc.

In the present disclosure, the heteroaryl, the heteroarylene, and the heterocycloalkyl, each independently, may contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arysilyl, a substituted or unsubstituted (C1-C30)alkyldi(C6-C30)arylsilyl, a substituted or unsubstituted tri(C6-C30)arysilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylamino, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

The plurality of host materials of the present disclosure comprises a first host material and a second host material, in which the first host material comprises the compound represented by formula 1 and the second host material comprises the compound represented by formula 2. According to one embodiment of the present disclosure, the compound represented by formula 1 and the compound represented by formula 2 are different from each other.

In formula 1, W represents a single bond, O, S, CR₆R₇, or N-L-R₈. According to one embodiment of the present disclosure, W represents a single bond, O, or S.

In formula 1, Y represents O, S, CR₆R₇, or N-L-R₈. According to one embodiment of the present disclosure, Y represents O, S, or N-L-R₈.

In formula 1, L, each independently, represents a single bond, a substituted or unsubstituted (C6-C30)arylene, or a substituted or unsubstituted (3- to 30-membered)heteroarylene. According to one embodiment of the present disclosure, L, each independently, represents a single bond, a substituted or unsubstituted (C6-C25)arylene, or a substituted or unsubstituted (5- to 25-membered)heteroarylene. According to another embodiment of the present disclosure, L, each independently, represents a single bond, an unsubstituted (C6-C18)arylene, or a (5- to 20-membered)heteroarylene unsubstituted or substituted with a (C6-C18)aryl(s). For example, L, each independently, may be a single bond, a phenylene, or a naphthylene.

In formula 1, R₁ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or adjacent ones of R₁ to R₈ may be linked to each other to form a ring(s). According to one embodiment of the present disclosure, at least one of R₁ to R₈ comprises a substituted or unsubstituted (3- to 30-membered)heteroaryl containing at least one nitrogen. For example, at least one of R₁ to R₈ comprises a substituted triazinyl, a substituted quinazolinyl, a substituted quinoxalinyl, a substituted quinolyl, a substituted carbazolyl, etc. According to another embodiment of the present disclosure, at least one of R₁ to R₆ comprises a substituted or unsubstituted (3- to 30-membered)heteroaryl containing at least one nitrogen. According to one embodiment of the present disclosure, R₁ to R₈, each independently, represent hydrogen, a substituted or unsubstituted (C6-C25)aryl, or a substituted or unsubstituted (5- to 25-membered)heteroaryl; or adjacent ones of R₁ to R₈ may be linked to each other to form a ring(s). According to another embodiment of the present disclosure, R₁ to R₈, each independently, represent hydrogen, a (C6-C18)aryl unsubstituted or substituted with a (3- to 30-membered)heteroaryl(s), or a (5- to 20-membered)heteroaryl unsubstituted or substituted with at least one of a (C6-C18)aryl(s) and a (5- to 20-membered)heteroaryl(s); or adjacent ones of R₁ to R₈ may be linked to each other to form a ring(s). For example, R₁, R₂, and R₅, each independently, may be hydrogen, a phenyl, or a triazinyl substituted with a phenyl(s); R₈ may be hydrogen, a phenyl, a carbazolyl substituted with a phenyl(s), or a triazinyl substituted with at least one of a phenyl(s), a biphenyl(s), and a dibenzofuranyl(s), or two adjacent R₃'s may be linked to each other to form a benzene ring(s); R₄ may be hydrogen, a substituted or unsubstituted phenyl, a substituted naphthyl, a substituted pyridyl, a substituted triazinyl, a quinolyl substituted with a phenyl(s), a quinazolyl substituted with a phenyl(s), a quinoxalinyl substituted with a phenyl(s), or a carbazolyl substituted with a phenyl(s), or two adjacent R₄'s may be linked to each other to form a benzene ring(s), in which the substituent(s) of the substituted phenyl, the substituted naphthyl, and the substituted pyridyl, each independently, may be a diphenyltriazinyl, a phenylbiphenyltriazinyl, or a phenylnaphthyltriazinyl, and the substituent(s) of the substituted triazinyl may be at least one of a phenyl, a naphthyl, a biphenyl, a terphenyl, a naphthylphenyl, a phenylnaphthyl, and a dibenzofuranyl; R₈ may be an unsubstituted phenyl, an unsubstituted naphthyl, an unsubstituted biphenyl, a substituted triazinyl, a substituted quinazolinyl, a substituted quinoxalinyl, or an unsubstituted dibenzofuranyl, in which the substituent(s) of the substituted triazinyl, the substituted quinazolinyl, and the substituted quinoxalinyl, each independently, may be at least one of a phenyl, a naphthyl, a biphenyl, and a dibenzofuranyl.

In formula 1, a to d, each independently, represent an integer of 1 to 4; and e represents an integer of 1 or 2; where each of a to e is an integer of 2 or more, each of R₁, each of R₂, each of R₃, each of R₄, and each of R₅ may be the same or different. According to one embodiment of the present disclosure, a, b, and e may be an integer of 1; and c and d may be an integer of 1 or 2.

According to one embodiment of the present disclosure, the formula 1 may be represented by at least one of the following formulas 1-1 to 1-4.

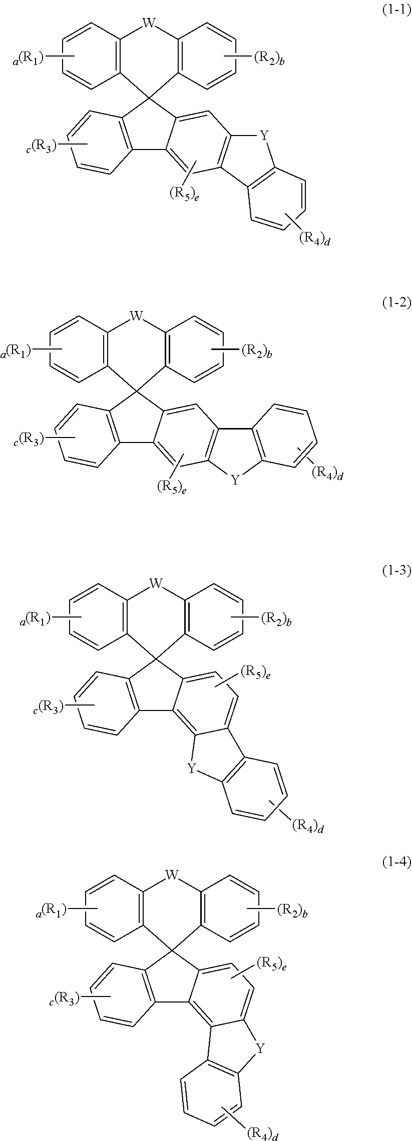

In formulas 1-1 to 1-4, W, Y, R₁ to R₅, and a to e are as defined in formula 1.

In formula 2, L represents a single bond, or a substituted or unsubstituted (C6-C30)arylene. According to one embodiment of the present disclosure, L₁ represents a single bond, or a substituted or unsubstituted (C6-C25)arylene. According to another embodiment of the present disclosure, L represents a single bond, or an unsubstituted (C6-C18)arylene. For example, L₁ may be a single bond, a phenylene, a naphthylene, or a biphenylene.

In formula 2, Ar₁ represents a substituted or unsubstituted (C6-C30)aryl, or a substituted or unsubstituted (3- to 30-membered)heteroaryl containing at least one nitrogen (N). According to one embodiment of the present disclosure, Ar₁ represents a substituted or unsubstituted (C6-C30)aryl, or a substituted or unsubstituted (13- to 30-membered)heteroaryl containing at least one nitrogen (N). According to another embodiment of the present disclosure, Ar₁ represents an unsubstituted (C6-C25)aryl, or a (13- to 25-membered)heteroaryl unsubstituted or substituted with a (C6-C18)aryl(s) and containing at least one nitrogen (N). For example, Ar₁ may be a substituted or unsubstituted phenyl, a naphthyl, a biphenyl, a terphenyl, a triphenylenyl, a dimethylfluorenyl, a diphenylfluorenyl, or a carbazolyl unsubstituted or substituted with a phenyl(s), in which the substituent of the substituted phenyl may be at least one of a cyano(s), a methyl(s), and a triphenylsilyl(s).

In formula 2, R₉ and R₁₀, each independently, represent hydrogen, deuterium, a halogen, a cyano, a substituted or unsubstituted (C1-C30)alkyl, a substituted or unsubstituted (C6-C30)aryl, a substituted or unsubstituted (3- to 50-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or adjacent ones of R₉ and R₁₀ may be linked to each other to form a ring(s), in which any one of R₁₀ represents a substituted or unsubstituted (3- to 50-membered)heteroaryl, or two or more adjacent ones of R₁₀'s may be linked to each other to form a ring(s). If R₉ or R₁₀ represents a substituted (C6-C30)aryl, the substituent of the substituted aryl may not be a (3- to 30-membered)heteroaryl, for example, may not be a carbazolyl or a dibenzofuranyl. If R₉ or R₁₀ represents a substituted or unsubstituted carbazolyl, R₉ or R₁₀ may be linked to the backbone at a 9 position of the carbazolyl. According to one embodiment of the present disclosure, if R₉ or R₁₀ forms a substituted ring, the substituent of the substituted ring does not comprise a triazinyl. For example, the substituent of the substituted ring may not be a diphenyltriazinyl, or a phenyl substituted with a diphenyltriazinyl(s). According to one embodiment of the present disclosure, R₉ and R₁₀, each independently, represent hydrogen, a substituted or unsubstituted (C6-C25)aryl, or a substituted or unsubstituted (5- to 40-membered)heteroaryl; or adjacent ones of R₉ and R₁₀ may be linked to each other to form a ring(s). According to another embodiment of the present disclosure, one of R₉ and R₁₀ represents hydrogen, an unsubstituted (C6-C18)aryl, or a (5- to 20-membered)heteroaryl unsubstituted or substituted with a (C6-C18)aryl(s), and the other of R₉ and R₁₀ represents a (5- to 35-membered)heteroaryl unsubstituted or substituted with a (C6-C18)aryl(s) or a (5- to 20-membered)heteroaryl(s), or adjacent ones may be linked to each other to form a ring(s). For example, R₉ may be hydrogen, a phenyl, or a carbazolyl substituted with a phenyl(s); and R₁₀ may be a substituted or unsubstituted carbazolyl, or a 33-membered heteroaryl containing nitrogen and oxygen and substituted with a phenyl(s), or two or more adjacent ones of R₁₀ may be linked to each other to form a substituted indole ring or a substituted or unsubstituted spiro[indene-xanthene] ring, in which the substituent of the substituted carbazolyl may be at least one of a phenyl, a naphthyl, a biphenyl, a terphenyl, a triphenylenyl, and a pyridyl substituted with a phenyl(s), and the substituent of the substituted indole ring may be at least one of a phenyl, a biphenyl, a naphthyl, a naphthylphenyl, a phenylnaphthyl, a terphenyl, a triphenylenyl, a pyridyl substituted with a phenyl(s), and a pyrimidinyl substituted with a phenyl(s), and the substituent of the substituted spiro[indene-xanthene] ring may be a carbazolyl substituted with a phenyl(s).

In formula 2, f and g, each independently, represent an integer of 0 to 4; where each of f and g is an integer of 2 or more, each of R₉ and each of R₁₀ may be the same or different. According to one embodiment of the present disclosure, f and g are not zero (0) at the same time. According to another embodiment of the present disclosure, f may be an integer of 1, and g may be an integer of 1 or 2.

According to one embodiment of the present disclosure, the formula 2 may be represented by at least one of the following formulas 2-1 to 2-3.

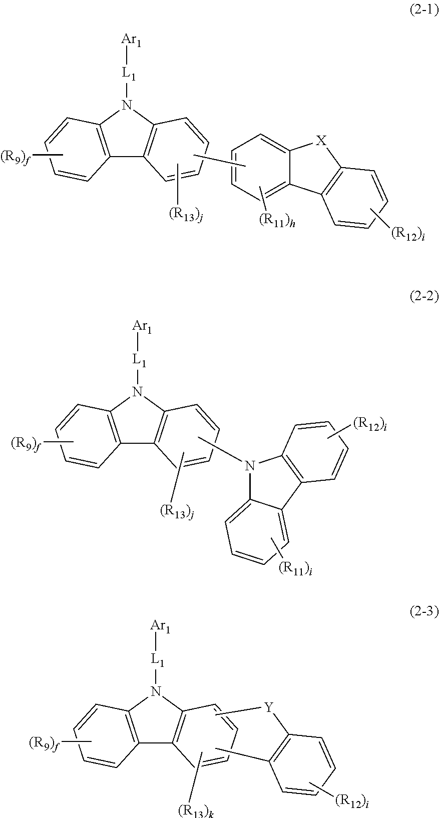

In formulas 2-1 and 2-3, X and Y, each independently, represent N-L₂-Ar₂, O, S, or CR₁₄CR₁₅. According to one embodiment of the present disclosure, X represents O, S, or CR₁₄CR₁₅, and Y represents N-L₂-Ar₂, O, S, or CR₁₄CR₁₅. According to another embodiment of the present disclosure, X and Y, each independently, represent N-L₂-Ar₂ or CR₁₄CR₁₅. For example, Y may be N-L₂-Ar₂.

L₂ represents a single bond, or a substituted or unsubstituted (C6-C30)arylene. According to one embodiment of the present disclosure, L₂ represents a single bond, or an unsubstituted (C6-C25)arylene. For example, L₂ may be a single bond, a phenylene, a naphthylene, or a biphenylene.

Ar₂ represents a substituted or unsubstituted (C6-C30)aryl, or a substituted or unsubstituted (3- to 30-membered)heteroaryl containing at least one nitrogen (N). According to one embodiment of the present disclosure, Ar₂ represents a substituted or unsubstituted (C6-C30)aryl. According to another embodiment of the present disclosure, Ar₂ represents a substituted or unsubstituted (C6-C25)aryl. According to further embodiment of the present disclosure, Ar₂ represents a (C6-C18)aryl unsubstituted or substituted with at least one of a cyano(s), a (C1-C6)alkyl(s), and a tri(C6-C18)arylsilyl(s). For example, Ar₂ may be a substituted phenyl, a naphthyl, a biphenyl, a terphenyl, a triphenylenyl, a dimethylfluorenyl, or a diphenylfluorenyl, in which the substituent of the substituted phenyl may be at least one of a cyano(s), a methyl(s), and a triphenylsilyl(s).

In formulas 2-1 to 2-3, R₁₁ to R₁₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or two or more adjacent R₁₁'s may be linked to each other to form a ring(s), two or more adjacent R₁₂'s may be linked to each other to form a ring(s), two or more adjacent R₁₃'s may be linked to each other to form a ring(s), and R₁₄ and R₁₅ may be linked to each other to form a ring(s). According to one embodiment of the present disclosure, in formula 2-3, R₁₂ is not a triazinyl. According to one embodiment of the present disclosure, R₁₁ to R₁₅, each independently, represent hydrogen, a substituted or unsubstituted (C1-C20)alkyl, a substituted or unsubstituted (C6-C25)aryl, or a substituted or unsubstituted (5- to 20-membered)heteroaryl; or two or more adjacent R₁₂'s may be linked to each other to form a ring(s), and R₁₄ and R₁₅ may be linked to each other to form a ring(s). According to another embodiment of the present disclosure, R₁₁ to R₁₅, each independently, represent hydrogen, an unsubstituted (C1-C10)alkyl, an unsubstituted (C6-C18)aryl, or a (5- to 18-membered)heteroaryl unsubstituted or substituted with an (C6-C18)aryl(s); or two or more adjacent R₁₂'s may be linked to each other to form a ring(s), and R₁₄ and R₁₅ may be linked to each other to form a ring(s). For example, R₁₁ and R₁₃ may be hydrogen; R₁₂ may be hydrogen, a phenyl, or a carbazolyl unsubstituted or substituted with a phenyl(s), or two or more adjacent R₁₂'s may be linked to each other to form a spiro[indene-xanthene] ring or an indole ring substituted with a phenyl(s); and R₁₄ and R₁₅ may be linked to each other to form a xanthene ring unsubstituted or substituted with a phenylcarbazolyl(s).

In formulas 2-1 to 2-3, i and l, each independently, represent an integer of 1 to 4; h and j, each independently, represent an integer of 1 to 3; and k represents an integer of 1 or 2; where each of h to l is an integer of 2 or more, each of R₁₁, each of R₁₂, and each of R₁₃ may be the same or different. According to one embodiment of the present disclosure, h, j, and k may be an integer of 1, and i may be an integer of 1 or 2.

In formulas 2-1 to 2-3, L₁, Ar₁, R₉ and f are as defined in formula 2.

According to one embodiment of the present disclosure, the formula 2 may be represented by the following formula 3.

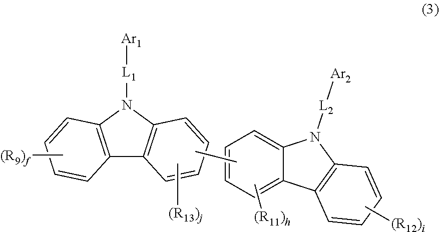

In formula 3, L₁ and L₂, each independently, represent a single bond, or a substituted or unsubstituted (C6-C30)arylene. According to one embodiment of the present disclosure, L₁ and L₂, each independently, represent a single bond, or a substituted or unsubstituted (C6-C25)arylene. According to another embodiment of the present disclosure, L₁ and L₂, each independently, represent a single bond, or an unsubstituted (C6-C18)arylene. For example, L₁ and L₂, each independently, may be a single bond, a phenylene, a naphthylene, or a biphenylene.

In formula 3, Ar₁ and Ar₂, each independently, represent a substituted or unsubstituted (C6-C30)aryl. According to one embodiment of the present disclosure, Ar₁ and Ar₂, each independently, represent a substituted or unsubstituted (C6-C25)aryl. According to another embodiment of the present disclosure, Ar₁ and Ar₂, each independently, represent a (C6-C25)aryl unsubstituted or substituted with at least one of a cyano(s), a (C1-C6)alkyl(s), and a tri(C6-C18)arylsilyl(s). For example, Ar₁ and Ar₂, each independently, may be a substituted or unsubstituted phenyl, a naphthyl, a biphenyl, a terphenyl, a triphenylenyl, a dimethylfluorenyl, or a diphenylfluorenyl, in which the substituent of the substituted phenyl may be at least one of a cyano(s), a methyl(s), and a triphenylsilyl(s).

In formula 3, R₉ and R₁₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or two or more adjacent R's may be linked to each other to form a ring(s), two or more adjacent R₁₁'s may be linked to each other to form a ring(s), two or more adjacent R₁₂'s may be linked to each other to form a ring(s), and two or more adjacent R₁₃'s may be linked to each other to form a ring(s). According to one embodiment of the present disclosure, R₉ and R₁₁ to R₁₃, each independently, represent hydrogen, a substituted or unsubstituted (C1-C20)alkyl, a substituted or unsubstituted (C6-C25)aryl, or a substituted or unsubstituted (5- to 25-membered)heteroaryl. According to another embodiment of the present disclosure, R₉ and R₁₁ to R₁₃, each independently, represent hydrogen, an unsubstituted (C1-C10)alkyl, an unsubstituted (C6-C18)aryl, or a (5- to 20-membered)heteroaryl unsubstituted or substituted with an (C6-C18)aryl(s). For example, R₉ and R₁₂, each independently, may be hydrogen, a methyl, a phenyl, a carbazolyl substituted with a phenyl(s); and R₁₁ and R₁₃ may be hydrogen.

In formula 3, f and i, each independently, represent an integer of 1 to 4; and h and j, each independently, represent an integer of 1 to 3; where f, h, i, and j is an integer of 2 or more, each of R₉, each of R₁₁, each of R₁₂, and each of R₁₃ may be the same or different.

Specifically, the compound represented by formula 1 may be exemplified by the following compounds, but is not limited thereto.

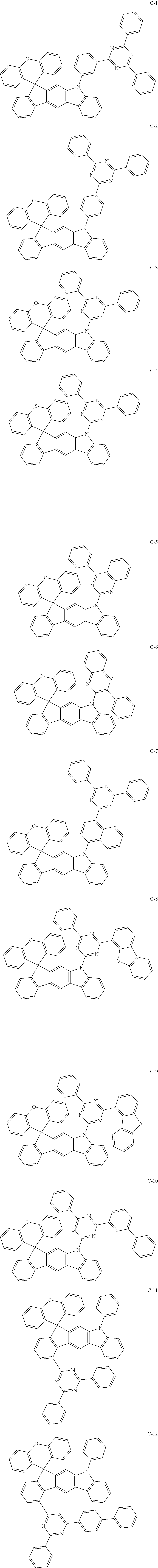

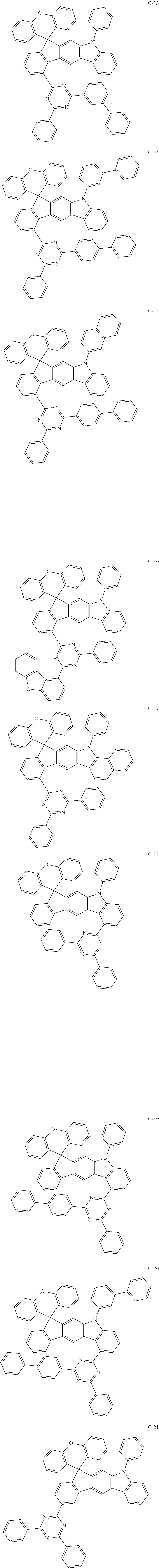

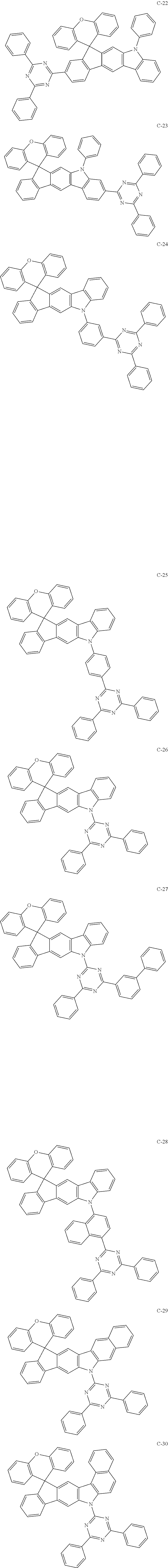

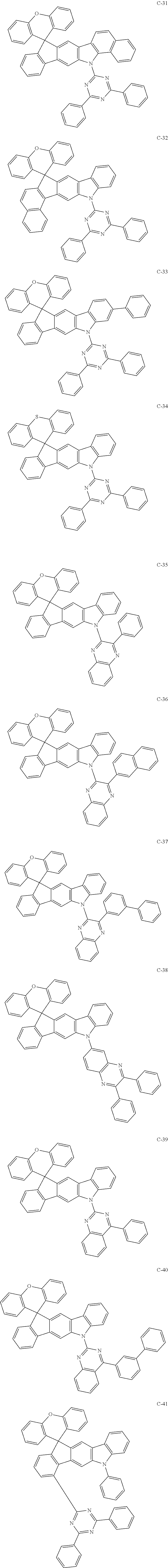

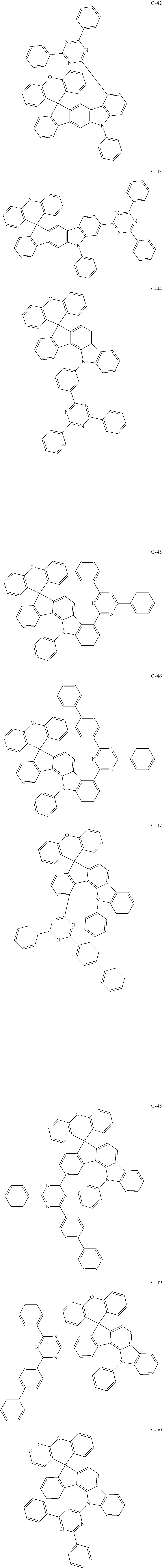

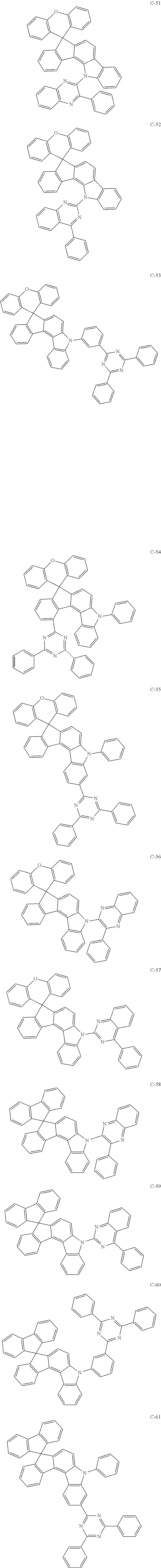

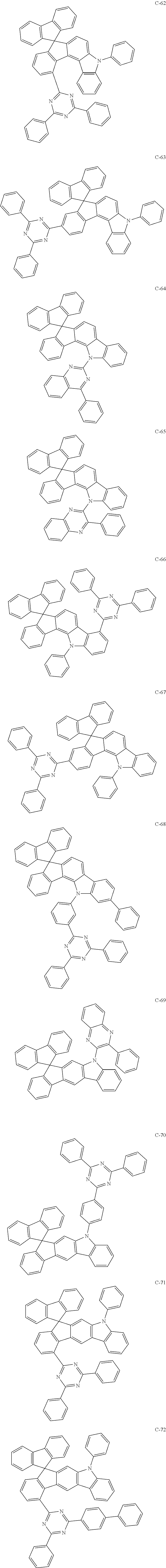

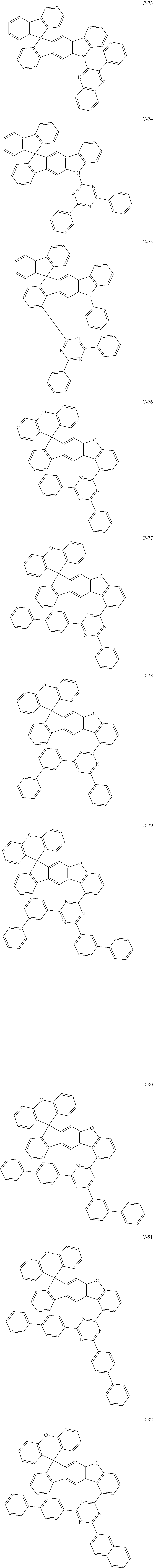

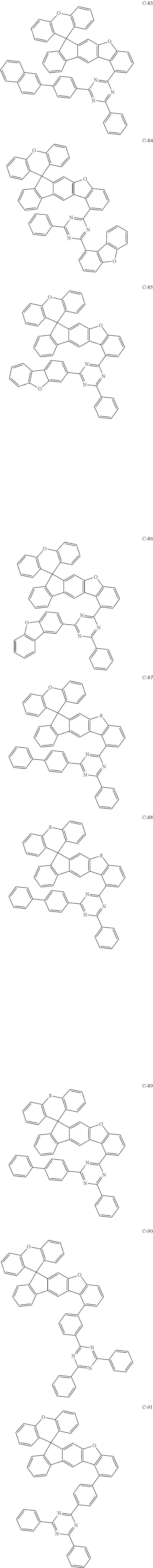

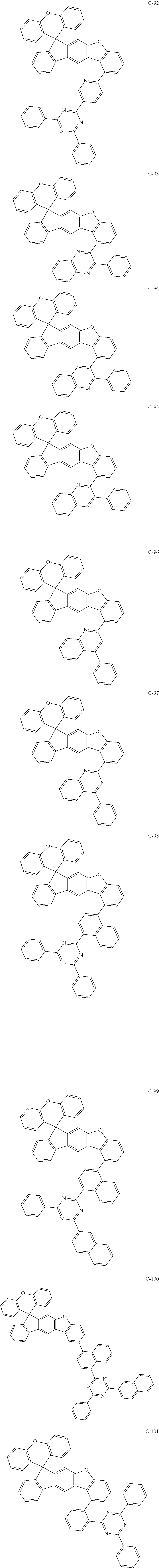

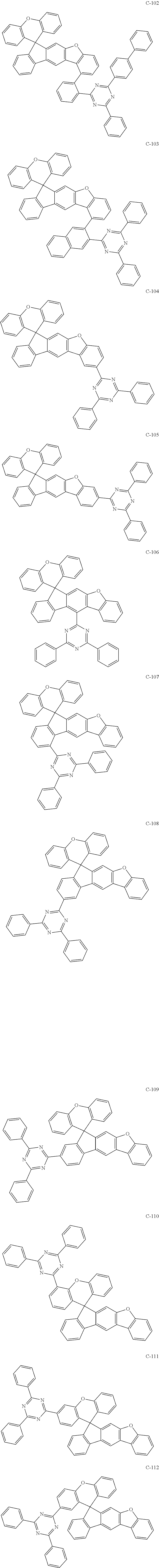

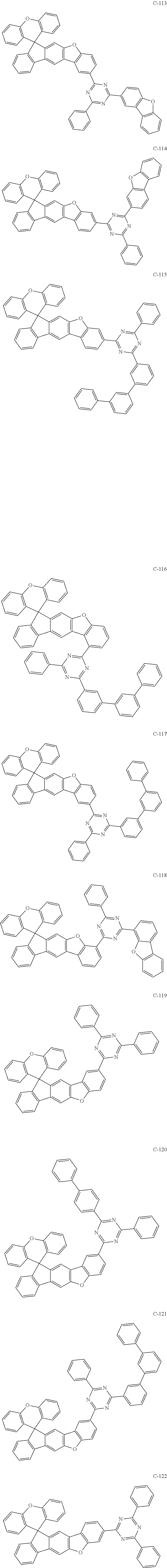

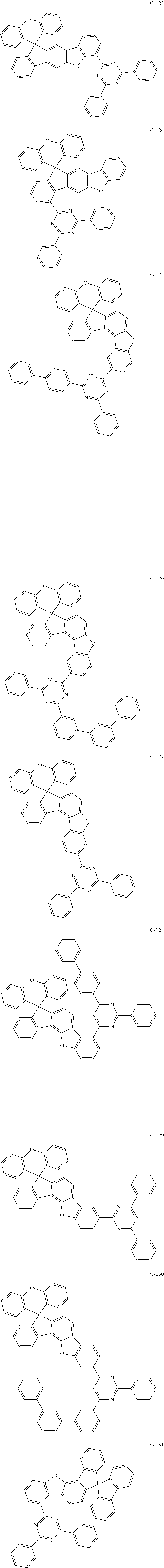

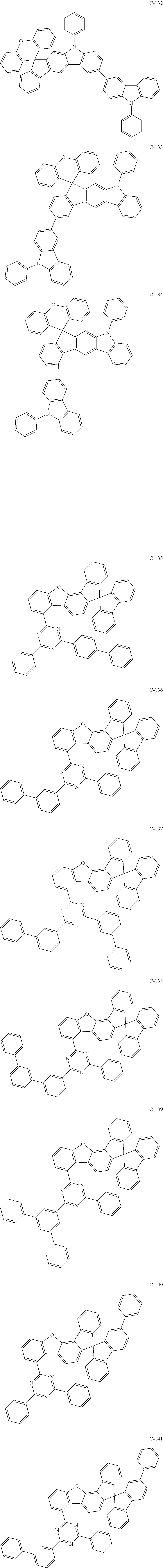

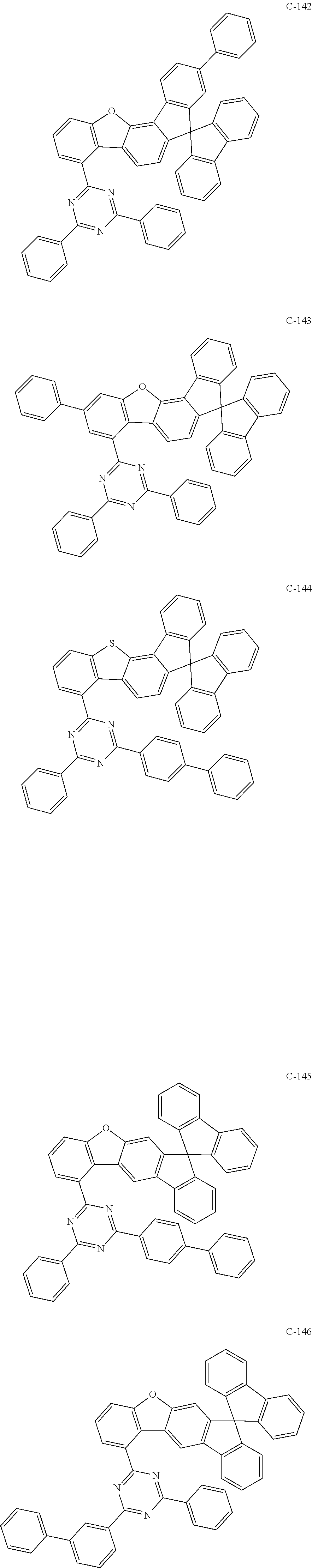

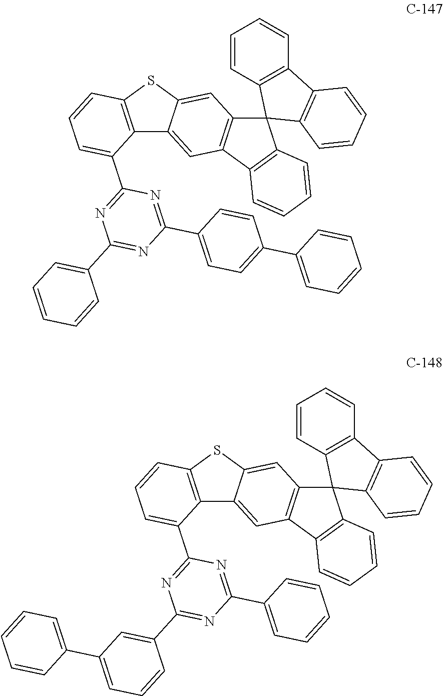 [SMILES: c1ccc(-c2ccc(-c3nc(-c4ccccc4)nc(-c4cccc5sc6cc7c(cc6c45)-c4ccccc4C74c5ccccc5-c5ccccc54)n3)cc2)cc1 ; c1ccc(-c2cccc(-c3nc(-c4ccccc4)nc(-c4cccc5sc6cc7c(cc6c45)-c4ccccc4C74c5ccccc5-c5ccccc54)n3)c2)cc1]

Specifically, the compound represented by formula 2 may be exemplified by the following compounds, but is not limited thereto.

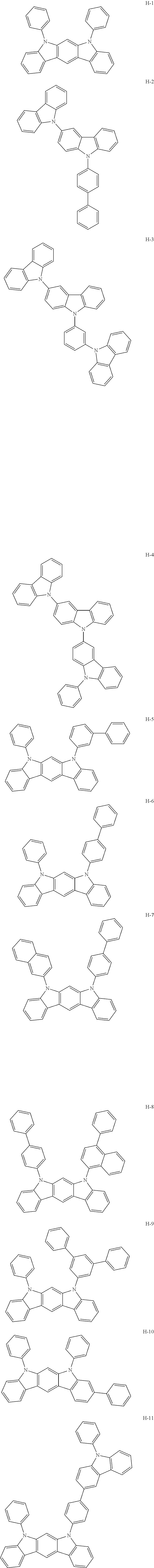

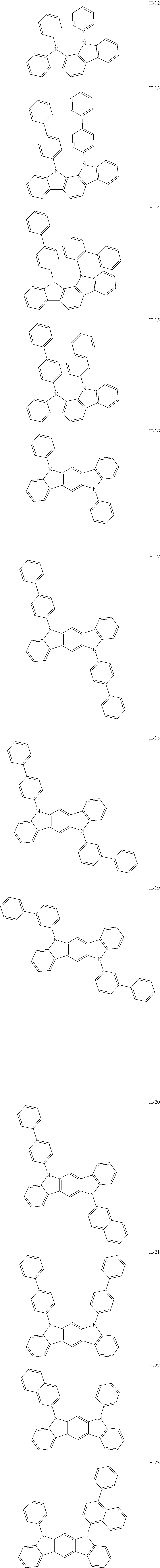

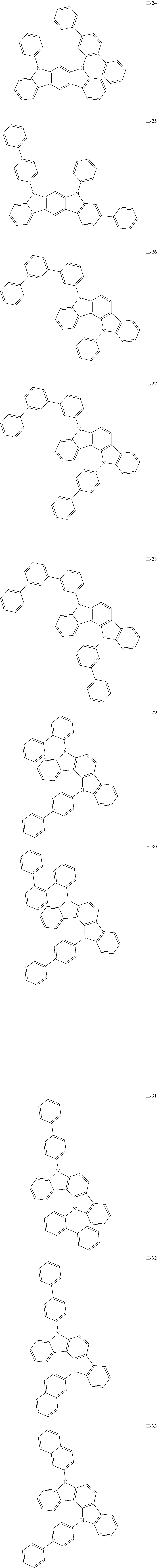

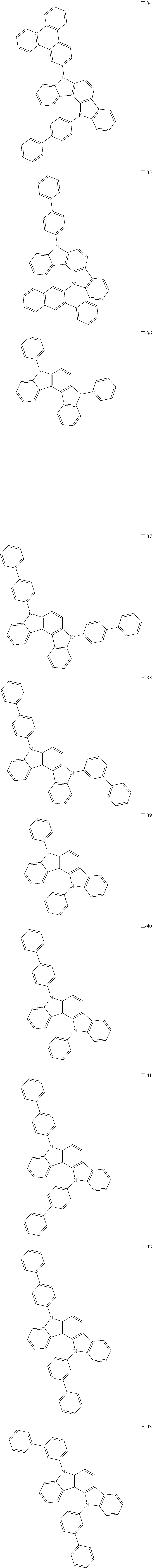

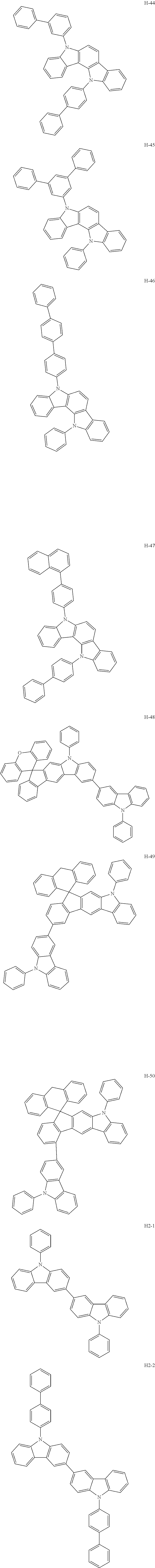

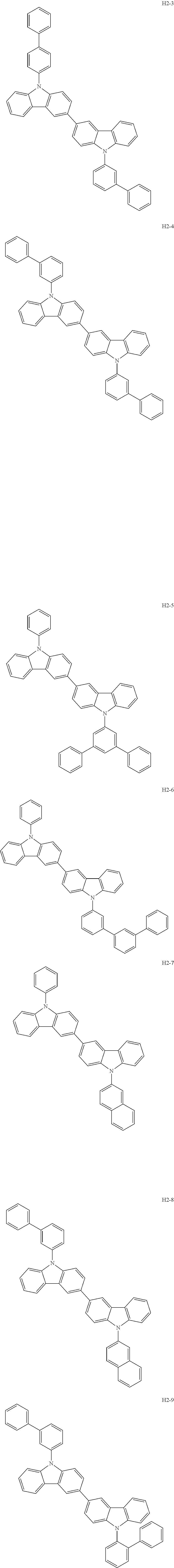

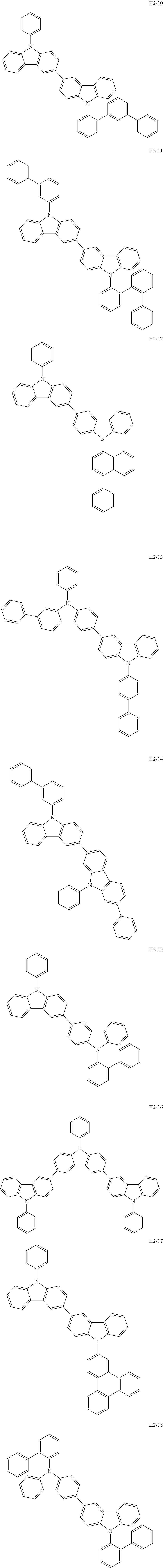

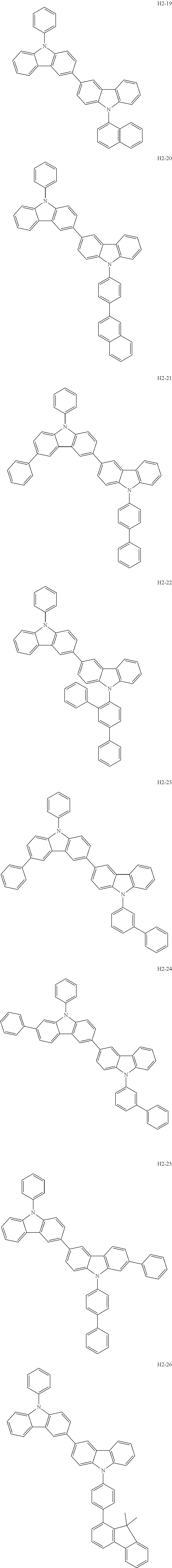

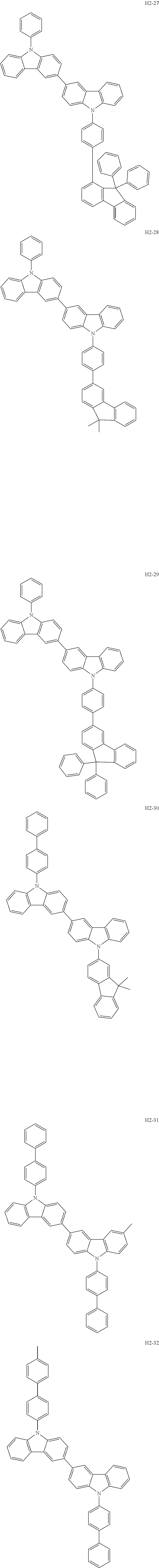

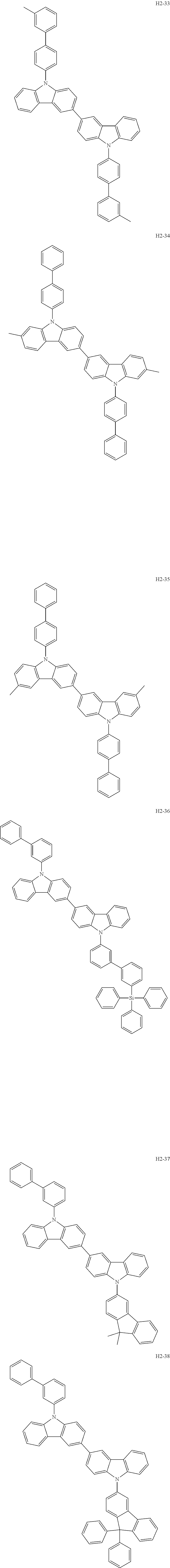

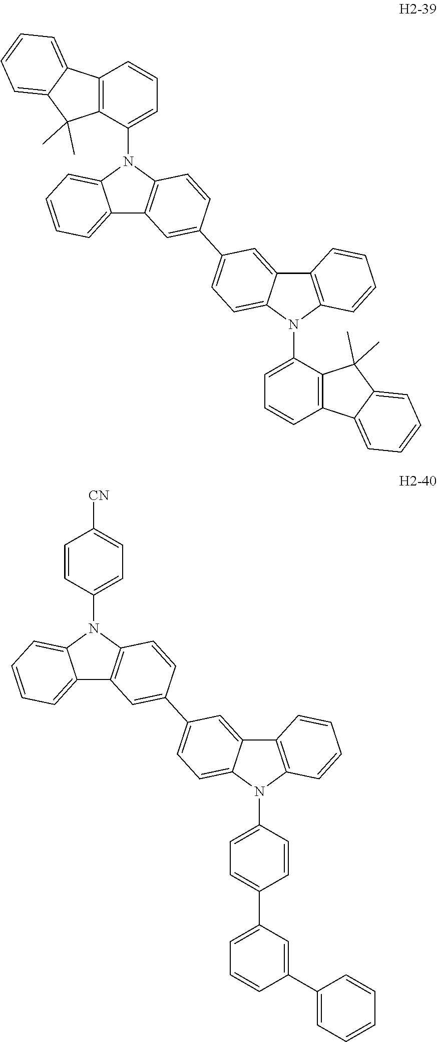 [SMILES: CC1(C)c2ccccc2-c2cccc(-n3c4ccccc4c4cc(-c5ccc6c(c5)c5ccccc5n6-c5cccc6c5C(C)(C)c5ccccc5-6)ccc43)c21 ; N#Cc1ccc(-n2c3ccccc3c3cc(-c4ccc5c(c4)c4ccccc4n5-c4ccc(-c5cccc(-c6ccccc6)c5)cc4)ccc32)cc1]

The combination of at least one of compounds C-1 to C-148 and at least one of compounds H-1 to H-50 and H2-1 to H2-40 may be used in an organic electroluminescent device. According to one embodiment of the present disclosure, the combination of at least one of compounds C-1 to C-148 and at least one of compounds H-1 to H-50 may be used in an organic electroluminescent device. According to another embodiment of the present disclosure, the combination of at least one of compounds C-11 to C-23, C-41 to C-43, C-45 to C-49, C-54, C-55, C-61 to C-63, C-66, C-67, C-71, C-72, C-75 to C-131, and C-133 to C-148 and at least one of compounds H2-1 to H2-40 may be used in an organic electroluminescent device.

The present disclosure provides an organic electroluminescent compound represented by formula 1, in which at least one of R₁ to R₅ comprises a substituted or unsubstituted (3- to 30-membered)heteroaryl containing at least one nitrogen. According to one embodiment of the present disclosure, if Y represents N-L-R₈, at least one of R₃ to R₅ comprises a substituted or unsubstituted (3- to 30-membered)heteroaryl containing at least one nitrogen. According to one embodiment of the present disclosure, if Y represents N-L-R₈, R₄ is not a 9-phenylcarbazolyl or a (9-carbazolyl)phenyl. According to one embodiment of the present disclosure, in the organic electroluminescent compound, at least one of R₁ to R₅ may comprise a (5- to 25-membered)heteroaryl containing at least one nitrogen and substituted with at least one of a (C6-C18)aryl(s) and a (3- to 30-membered)heteroaryl(s). According to another embodiment of the present disclosure, in the organic electroluminescent compound, at least one of R₁ to R₅ may comprise a substituted or unsubstituted (6-to 10-membered)heteroaryl containing at least one nitrogen. Specifically, in the organic electroluminescent compound, at least one of R₁ to R₅ may comprise a substituted or unsubstituted triazinyl, a substituted or unsubstituted quinolyl, a substituted or unsubstituted quinazolinyl, a substituted or unsubstituted quinoxalinyl, or a substituted or unsubstituted carbazolyl. More specifically, in the organic electroluminescent compound, at least one of R₁ to R₅ may comprise a substituted triazinyl, a quinolyl substituted with a phenyl(s), a quinazolinyl substituted with a phenyl(s), a quinoxalinyl substituted with a phenyl(s), or a carbazolyl substituted with a phenyl(s), in which the substituent of the substituted triazinyl may be at least one, preferably two, selected from the group consisting of a phenyl, a naphthyl, a biphenyl, a terphenyl, a naphthylphenyl, a phenylnaphthyl, and a dibenzofuranyl. For example, in the organic electroluminescent compound, at least one of R₁ to R₅ may comprise a substituted triazinyl, a phenyl substituted with a substituted triazinyl(s), a naphthyl substituted with a substituted triazinyl(s), a pyridyl substituted with a substituted triazinyl(s), a quinolyl substituted with a phenyl(s), a quinazolinyl substituted with a phenyl(s), a quinoxalinyl substituted with a phenyl(s), or a carbazolyl substituted with a phenyl(s), in which the substituent of the substituted triazinyl may be two selected from the group consisting of a phenyl, a naphthyl, a biphenyl, a terphenyl, a naphthylphenyl, a phenylnaphthyl, and a dibenzofuranyl, and the two substituents may be the same or different.

According to one embodiment of the present disclosure, in the organic electroluminescent compound, R₈ may be an unsubstituted (C6-C18)aryl, for example, a phenyl, a naphthyl, a biphenyl, etc.

The organic electroluminescent compound may specifically be exemplified as compounds C-11 to C-23, C-41 to C-43, C-45 to C-49, C-54, C-55, C-61 to C-63, C-66, C-67, C-71, C-72, C-75 to C-131, and C-133 to C-148, but is not limited thereto.

The present disclosure may provide an organic electroluminescent device comprising the organic electroluminescent compound, in which the organic electroluminescent compound may be comprised in a light-emitting layer. In addition, the present disclosure may provide a plurality of host materials comprising the organic electroluminescent compound as a first host material and the compound represented by formula 2, for example, the compound represented by formula 3, as a second host material, and an organic electroluminescent device comprising the same.

The compounds represented by formulas 1 to 3 and 2-1 to 2-3 according to the present disclosure may be prepared by a synthetic method known to one skilled in the art, and for example, the compound represented by formula 1 may be prepared as shown in the following reaction schemes 1 and 2, and the compound represented by formula 2 may be prepared as shown in the following reaction schemes 3 to 5, but is not limited thereto.

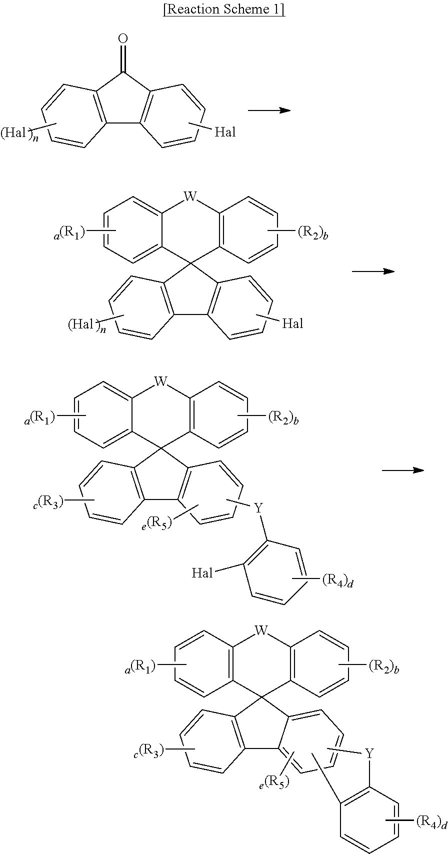

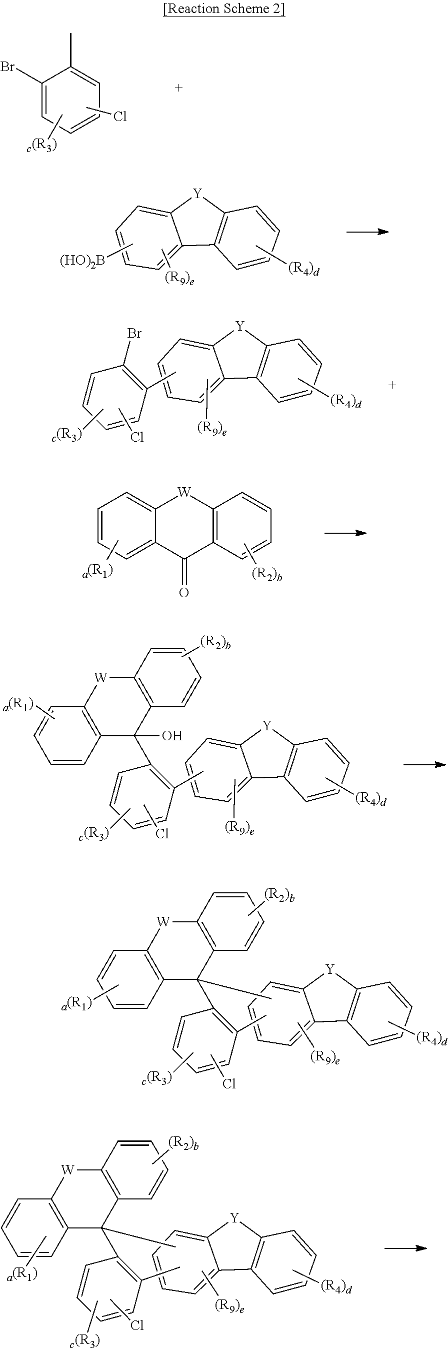

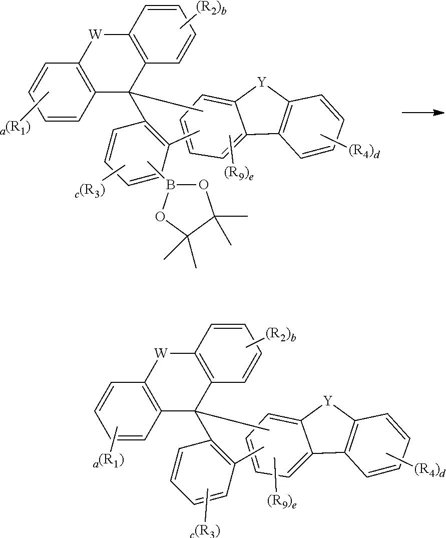

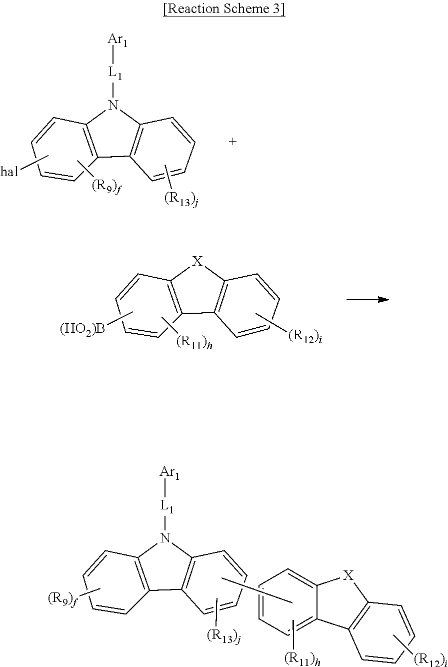

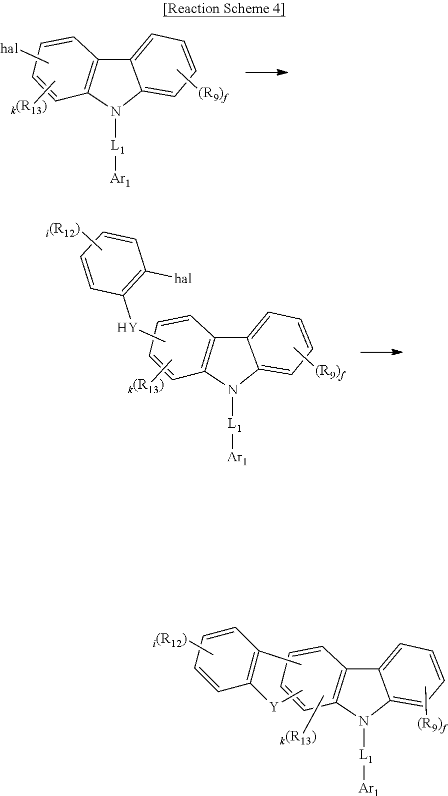

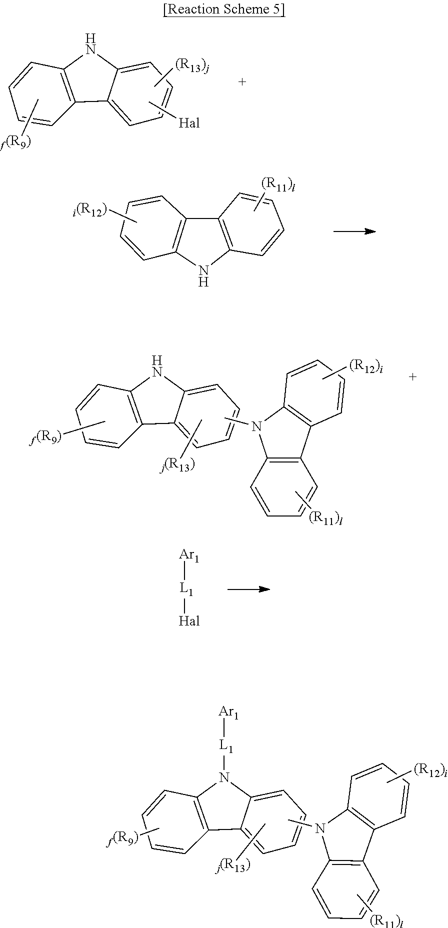

In reaction schemes 1 to 5, W, Y, X, R₁ to R₅, L₁, Ar₁, R₉, R₁₁ to R₁₃, a to f, and h to l are as defined in formulas 1, 2, and 2-1 to 2-3, and Hal represents I, Br, Cl, ONf (nonafluorobutanesulfonyl) or OTf (triflate).

Although illustrative synthesis examples of the compounds represented by formulas 1 and 2 of the present disclosure are described above, one skilled in the art will be able to readily understand that all of them are based on a Buchwald-Hartwig cross-coupling reaction, an N-arylation reaction, a Miyaura borylation reaction, a Suzuki cross-coupling reaction, a Pd(II)-catalyzed oxidative cyclization reaction, a Heck reaction, a Cyclic Dehydration reaction, an SN₁ substitution reaction, an SN₂ substitution reaction, a Phosphine-mediated reductive cyclization reaction, a Ullmann reaction, a Wittig reaction, etc., and the reactions above proceed even when substituents which are defined in formulas 1 and 2 above, but are not specified in the specific synthesis examples, are bonded.

The organic electroluminescent device according to the present disclosure may comprise an anode, a cathode, and at least one organic layer between the anode and cathode in which the organic layer may comprise a plurality of organic electroluminescent materials, including the compound represented by formula 1 as the first organic electroluminescent material, and the compound represented by formula 2 as the second organic electroluminescent material. According to one embodiment of the present disclosure, the organic electroluminescent device according to the present disclosure may comprise an anode, a cathode, and at least one light-emitting layer between the anode and cathode in which the light-emitting layer may comprise a plurality of host materials including the compound represented by formula 1 as the first host material, and the compound represented by formula 2 as the second host material.

The light-emitting layer includes a host and a dopant, in which the host includes a plurality of host materials, in which the plurality of host materials comprise a first host material and a second host material. The first host material may consist of the compound represented by formula 1 alone or may consist of at least one the compound represented by formula 1, and may further include conventional materials included in organic electroluminescent devices. The second host material may consist of the compound represented by formula 2 alone or may consist of at least one the compound represented by formula 2, and may further include conventional materials included in organic electroluminescent devices. The weight ratio of the first host compound and the second host compound is about 1:99 to about 99:1, preferably about 10:90 to about 90:10, more preferably about 30:70 to about 70:30, even more preferably about 40:60 to about 60:40, and still more preferably about 50:50.

Herein, the light-emitting layer is a layer from which light is emitted, and may be a single layer or a multi-layer of which two or more layers are stacked. All of the first host material and the second host material may be included in one layer, or the first host material and the second host material may be included in respective different light-emitting layers. According to one embodiment of the present disclosure, the doping concentration of the dopant compound with respect to the host compound in the light-emitting layer may be less than about 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound besides the plurality of host materials of the present disclosure as at least one of a hole injection material, a hole transport material, a hole auxiliary material, a light-emitting material, a light-emitting auxiliary material, and an electron blocking material. Further, according to one embodiment of the present disclosure, the organic electroluminescent device of the present disclosure may further comprise an azine-based compound besides the plurality of host materials of the present disclosure as at least one of an electron transport material, an electron injection material, an electron buffer material, and a hole blocking material.

In the organic electroluminescent device of the present disclosure, a hole injection layer, a hole transport layer, an electron blocking layer, or a combination thereof can be used between the anode and the light-emitting layer. The hole injection layer may be multilayers in order to lower the hole injection barrier (or hole injection voltage) from the anode to the hole transport layer or the electron blocking layer, wherein each of the multilayers may use two compounds simultaneously. The hole transport layer or the electron blocking layer may also be multilayers.

In addition, an electron buffer layer, a hole blocking layer, an electron transport layer, an electron injection layer, or a combination thereof can be used between the light-emitting layer and the cathode. The electron buffer layer may be multilayers in order to control the electron injection and improve the interfacial properties between the light-emitting layer and the electron injection layer, wherein each of the multilayers may use two compounds simultaneously. The hole blocking layer or the electron transport layer may also be multilayers, wherein each of the multilayers may use a plurality of compounds.

The dopant comprised in the organic electroluminescent device of the present disclosure may be at least one phosphorescent or fluorescent dopant, and is preferably at least one phosphorescent dopant. The phosphorescent dopant material applied to the organic electroluminescent device of the present disclosure is not particularly limited, but may be preferably selected from the metallated complex compounds of iridium (Ir), osmium (Os), copper (Cu), and platinum (Pt), more preferably selected from ortho-metallated complex compounds of iridium (Ir), osmium (Os), copper (Cu), and platinum (Pt), and even more preferably ortho-metallated iridium complex compounds.

The dopant comprised in the organic electroluminescent device of the present disclosure may comprise a compound represented by the following formula 101, but is not limited thereto.

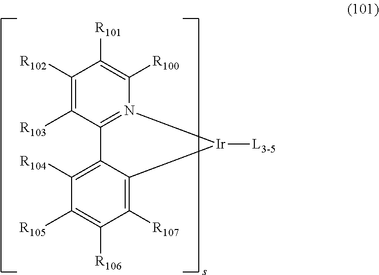

In formula 101, L is selected from the following structures 1 and 2:

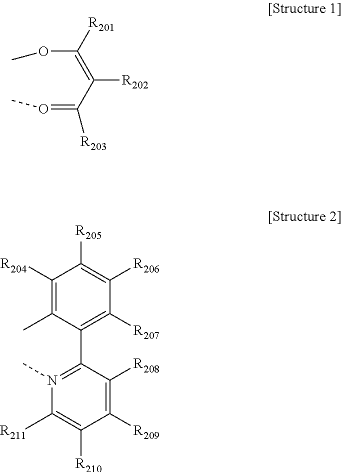

R₁₀₀ to R₁₀₃, each independently, represent hydrogen, deuterium, a halogen, a (C1-C30)alkyl unsubstituted or substituted with deuterium(s) or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent to form a ring(s), e.g., a substituted or unsubstituted, quinoline, benzofuropyridine, benzothienopyridine, indenopyridine, benzofuroquinoline, benzothienoquinoline, or indenoquinoline ring, together with pyridine;

R₁₀₄ to R₁₀₇, each independently, represent hydrogen, deuterium, a halogen, a (C1-C30)alkyl unsubstituted or substituted with deuterium(s) or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent to form a ring(s), e.g., a substituted or unsubstituted, naphthalene, fluorene, dibenzothiophene, dibenzofuran, indenopyridine, benzofuropyridine, or benzothienopyridine ring, together with benzene;

R₂₀₁ to R₂₁₁, each independently, represent hydrogen, deuterium, a halogen, a (C1-C30)alkyl unsubstituted or substituted with deuterium(s) or a halogen(s), a substituted or unsubstituted (C3-C30)cycloalkyl, or a substituted or unsubstituted (C6-C30)aryl; or may be linked to an adjacent substituent to form a ring(s); and

s represents an integer of 1 to 3.

The specific examples of the dopant compound are as follows, but are not limited thereto.

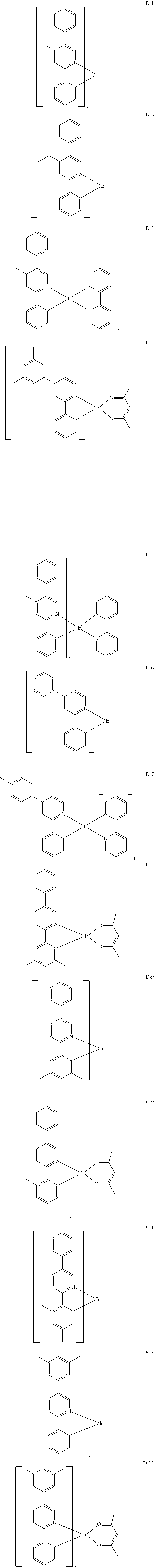

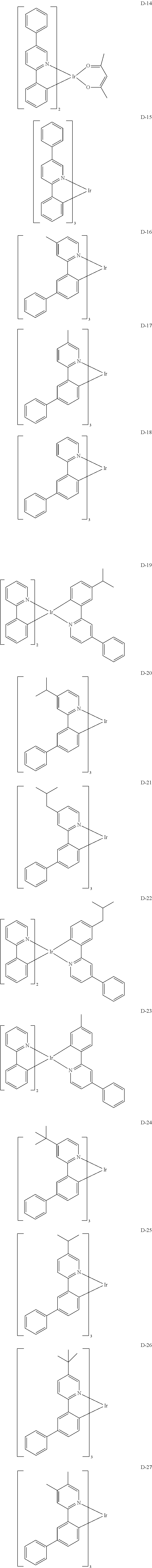

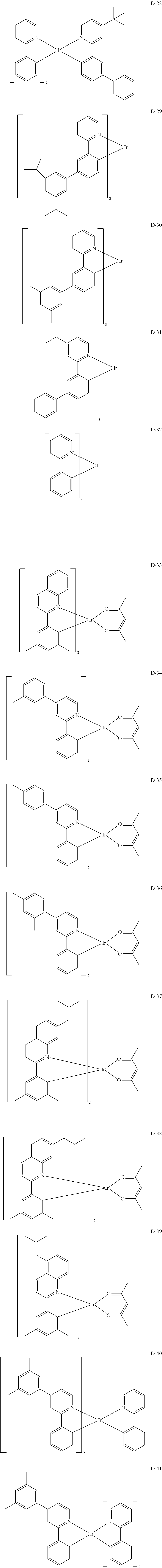

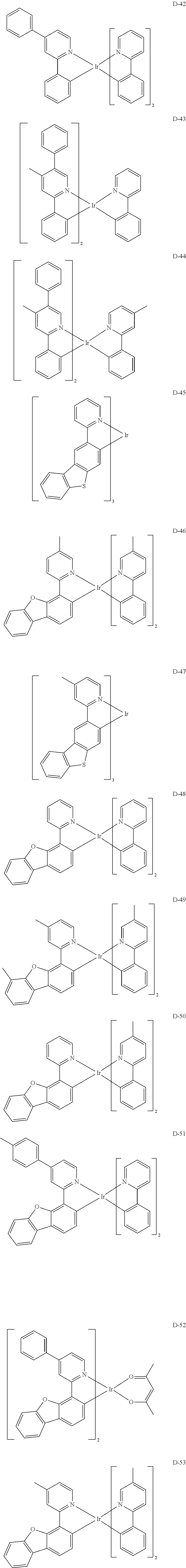

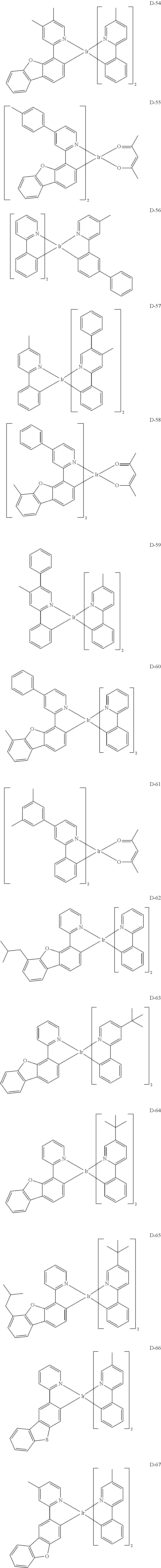

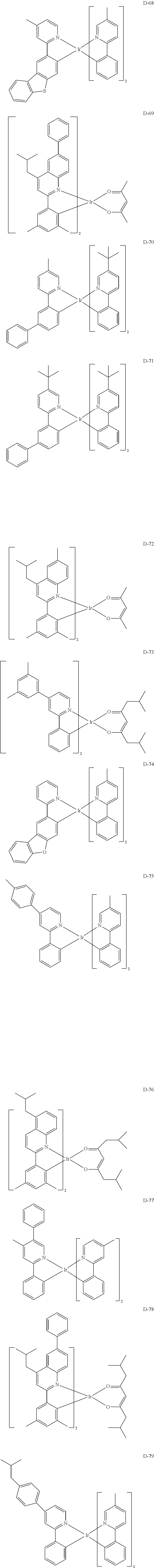

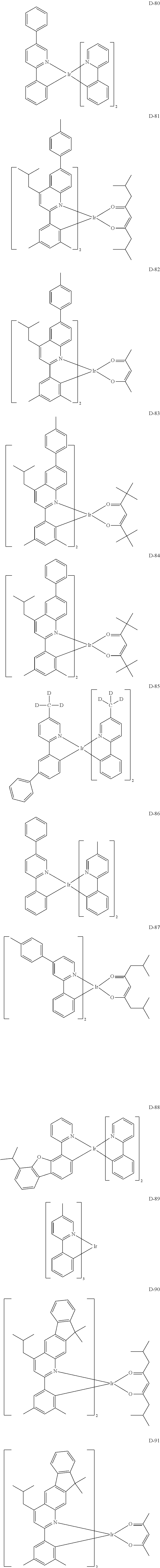

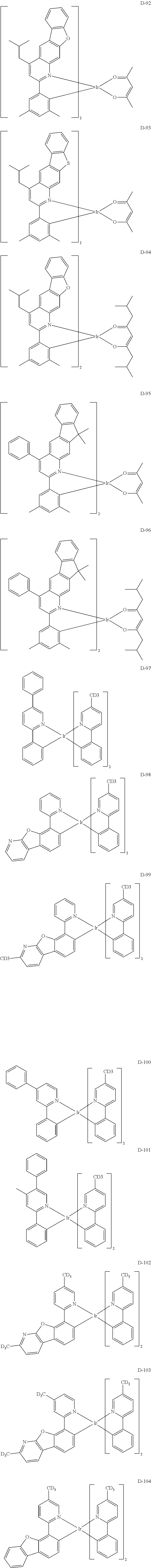

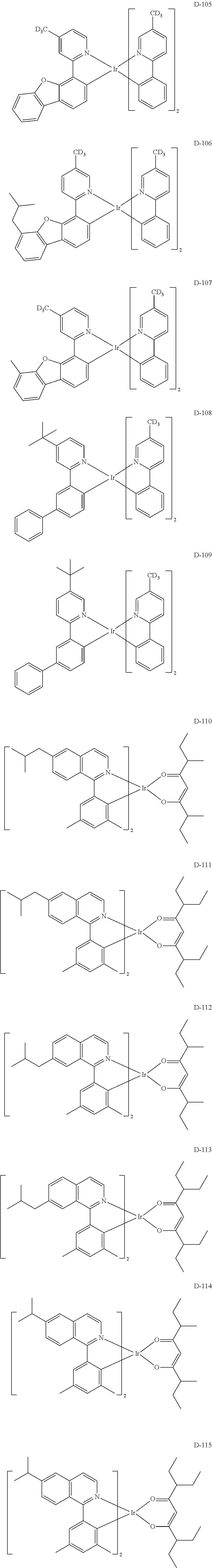

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any solvent where the materials forming each layer can be dissolved or diffused, and where there are no problems in film-formation capability.

The compounds represented by formulas 1 and 2 of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before evaporating them, and a current is applied to the cell to evaporate the materials. Further, if the first and the second host compounds are present in the same layer or different layers in an organic electroluminescent device, the two host compounds may individually form films. For example, the second host compound may be deposited after depositing the first host compound.

The present disclosure may provide a display device by using the organic electroluminescent compound represented by formula 1, or the plurality of host materials including the compound represented by formula 1 and the compound represented by formula 2. That is, it is possible to manufacture a display system or a lighting system by using the organic electroluminescent compound of the present disclosure or the plurality of host materials of the present disclosure. Specifically, it is possible to produce a display system, e.g., a display system for smart phones, tablets, notebooks, PCs, TVs, or cars; or a lighting system, e.g., an outdoor or indoor lighting system, by using the organic electroluminescent compound or the present disclosure or the plurality of host materials of the present disclosure.

Hereinafter, the preparation method of the compounds according to the present disclosure and the properties thereof will be explained in detail with reference to the representative compounds of the present disclosure. However, the present disclosure is not limited by the following examples.

### Example 1: Preparation of Compound C-11

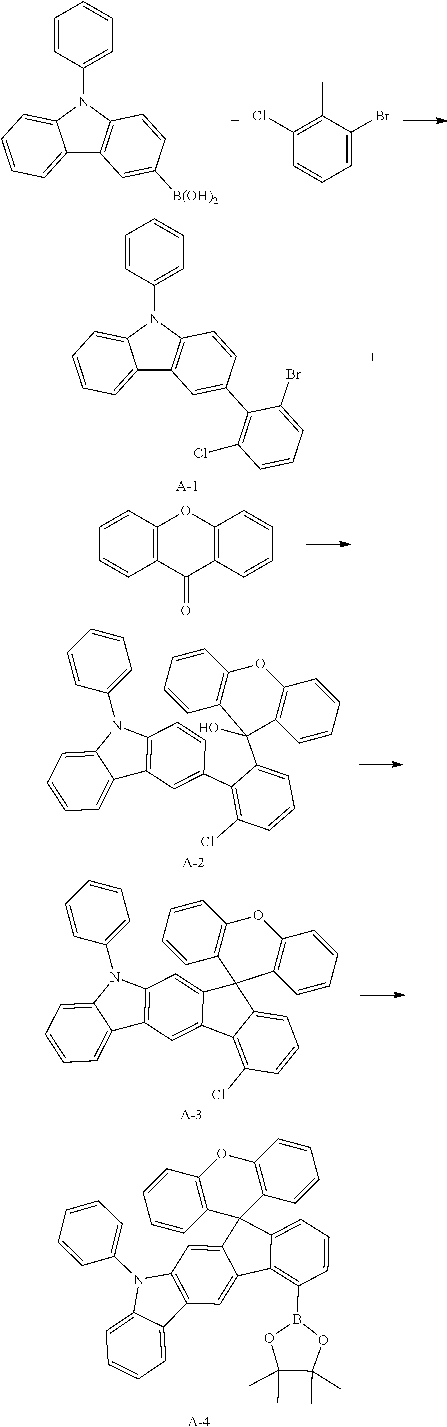

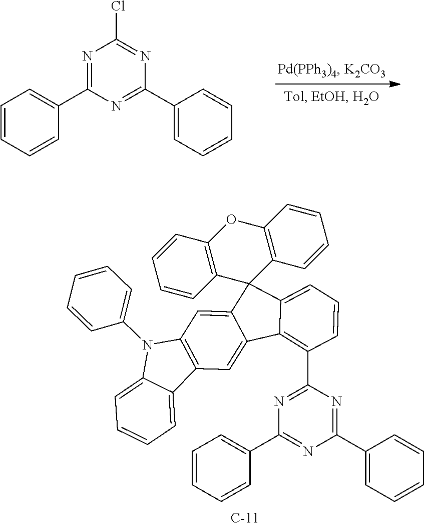

Synthesis of Compound A-1

9-phenyl-9H-carbazol-3-yl-boronic acid (51 g, 178 mmol), 1-bromo-3-chloro-2-iodobenzene (60 g, 189 mmol), Pd(PPh₃)₄ (2.1 g, 1.82 mmol), and potassium carbonate (65 g, 473 mmol) were dissolved in 170 mL of distilled water, 400 mL of toluene, and 400 mL of THE in flask, and the mixture was reacted at 70° C. for 4 hours. After completion of the reaction, an organic layer was extracted with ethyl acetate, and residual moisture was removed by using magnesium sulfate. The residue was dried and separated by column chromatography to obtain compound A-1 (46 g, yield: 57%).

Synthesis of Compound A-2

Compound A-1 (39 g, 90 mmol) and 350 mL of THF were added to a flask under a nitrogen atmosphere, and the mixture was cooled to −78° C. Next, n-butyl lithium (40 mL, 99 mmol) was slowly added dropwise to the mixture. Xanthon (16 g, 81 mmol) was then added dropwise, and the mixture was reacted for 2 hours. After completion of the reaction, water was added to the mixture to terminate the reaction, an organic layer was extracted with ethyl acetate, and residual moisture was removed by using magnesium sulfate. The residue was dried and separated by column chromatography to obtain compound A-2 (40 g, yield: 80%).

Synthesis of Compound A-3

Compound A-2 (46 g, 83 mmol), sulfuric acid (24 g, 250 mmol), and 850 mL of toluene were added to a flask under nitrogen atmosphere, and the mixture was reacted at 80° C. and for 2 hours. After completion of the reaction, the mixture was neutralized, an organic layer was extracted with ethyl acetate, and residual moisture was removed by using magnesium sulfate. The residue was dried and separated by column chromatography to obtain compound A-3 (19 g, yield: 50%).

Synthesis of Compound A-4

Compound A-3 (21 g, 40 mmol), bis(pinacolato)diboron (12 g, 50 mmol), Pd₂(dba)₃ (0.45 g, 0.5 mmol), potassium acetate (7.8 g, 80 mmol), tricyclohexylphosphine (0.43 g, 1.5 mmol), and 400 mL of toluene were added to a flask, and the mixture was reacted under reflux for 6 hours. After completion of the reaction, distilled water was added to the mixture to terminate the reaction, an organic layer was extracted with ethyl acetate, and residual moisture was removed by using magnesium sulfate. The residue was dried and separated by column chromatography to obtain compound A-4 (15 g, yield: 61%).

Synthesis of Compound C-11

In a flask, compound A-4 (5 g, 8 mmol), 2-chloro-4,6-diphenyl-1,3,5-triazine (3 g, 10 mmol), Pd(PPh₃)₄ (0.46 g, 0.4 mmol), and potassium carbonate (2.7 g, 20 mmol) were dissolved in 32 mL of toluene, 8 mL of distilled water and 8 mL of ethanol, and the mixture was reacted under reflux for 5 hours. After completion of the reaction, an organic layer was extracted with ethyl acetate and the residual moisture was removed by using magnesium sulfate. The residue was separated by column chromatography to obtain compound C-11 (2.1 g, yield: 36%).

| | MW | M.P. |
|---|---|---|
| C-11 | 565.63 | 272° C. |

### Example 2: Preparation of Compound C-12

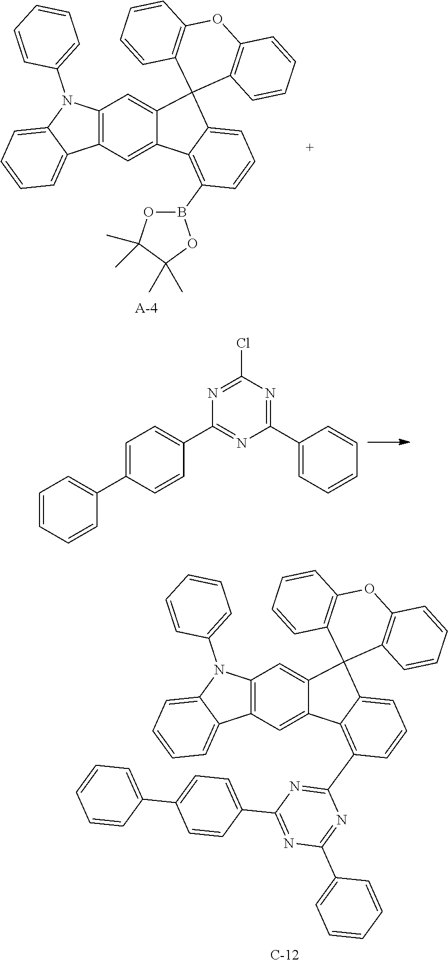

In a flask, compound A-4 (5 g, 8 mmol), 2-([1,1′-biphenyl]-4-yl)-4-chloro-6-phenyl-1,3,5-triazine (3.4 g, 10 mmol), Pd(PPh₃)₄ (0.46 g, 0.4 mmol), and potassium carbonate (2.7 g, 20 mmol) were dissolved in 32 mL of toluene, 8 mL of distilled water and 8 mL of ethanol, and the mixture was reacted under reflux for 5 hours. After completion of the reaction, an organic layer was extracted with ethyl acetate and the residual moisture was removed by using magnesium sulfate. The residue was separated by column chromatography to obtain compound C-12 (2.9 g, yield: 45%).

| | MW | M.P. |
|---|---|---|
| C-12 | 804.95 | 250° C. |

### Example 3: Preparation of Compound C-131

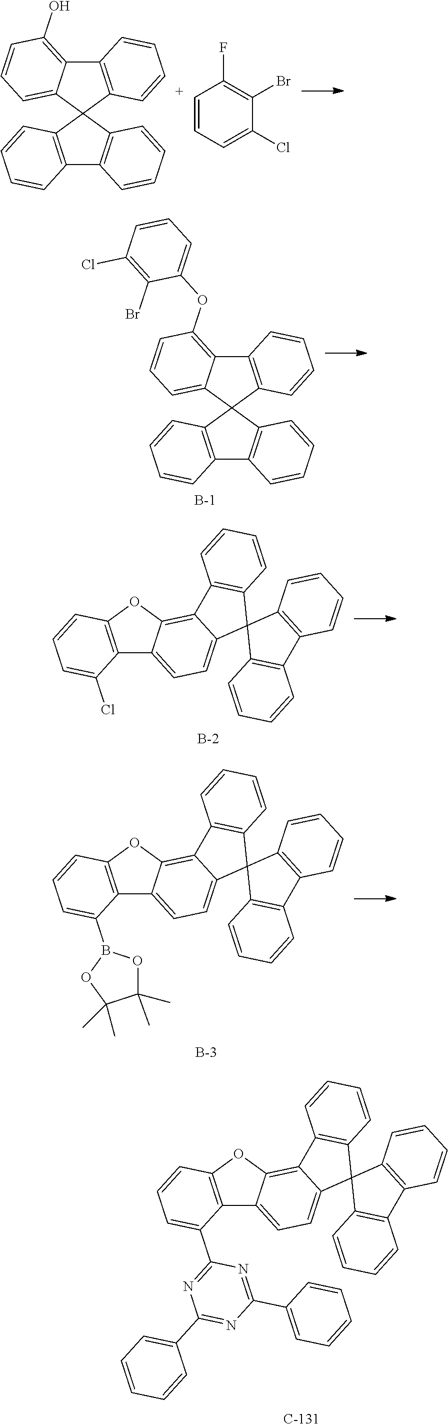

Synthesis of Compound B-1

9,9′-spirobi[9H-fluoren]-4-ol (114 g, 343 mmol), 2-bromo-1-chloro-3-fluorobenzene (144 g, 686 mmol), potassium carbonate (224 g, 686 mmol), and 1 L of N-methyl-2-pyrrolidone (NMP) were added to a flask, and the mixture was reacted at 150° C. for 3 hours. After completion of the reaction, an organic layer was extracted with ethyl acetate, and residual moisture was removed by using magnesium sulfate. The residue was dried and separated by column chromatography to obtain compound B-1 (155 g, yield: 86%).

Synthesis of Compound B-2

Compound B-1 (155 g, 300 mmol), Pd(OAc)₂ (3.3 g, 15 mmol), tricyclohexylphosphine (8.4 g, 30 mmol), potassium carbonate (124 g, 900 mmol), and 1.5 L of dimethylacetamide (DMAc) were added to a flask, and the mixture was reacted at 150° C. for 10 hours. After completion of the reaction, an organic layer was extracted with ethyl acetate, and residual moisture was removed by using magnesium sulfate. The residue was dried and separated by column chromatography to obtain compound B-2 (95 g, yield: 72%).

Synthesis of Compound B-3

Compound B-2 (35 g, 79 mmol), potassium acetate (15 g, 158 mmol), bis(pinacolato)diboron (30 g, 119 mmol), Pd(dba)₂ (0.9 g, 2%), tricyclohexylphosphine (0.9 g, 4 mol %), 18-crown-6 (0.3 g, 1 mol %), and 600 mL of toluene were added to a flask, and the mixture was reacted at 110° C. for 8 hours. After completion of the reaction, an organic layer was extracted with ethyl acetate, and residual moisture was removed by using magnesium sulfate. The residue was dried and separated by column chromatography to obtain compound B-3 (12 g, yield: 30%).

Synthesis of Compound C-131

Compound B-3 (5 g, 9.4 mmol), 2-chloro-4,6-diphenyl-1,3,5-triazine (3.5 g, 13 mmol), Pd(PPh₃)₄ (0.5 g, 0.47 mmol), potassium carbonate (3.2 g, 23 mmol), 50 mL of toluene, 12 mL of ethanol, and 12 mL of distilled water were added to a flask, and the mixture was reacted under reflux for 4 hours. After completion of the reaction, an organic layer was extracted with ethyl acetate and the residual moisture was removed by using magnesium sulfate. The residue was separated by column chromatography to obtain compound C-131 (5.2 g, yield: 87%).

| | MW | M.P. |
|---|---|---|
| C-131 | 637.74 | 166° C. |

### Example 4: Preparation of Compound C-76

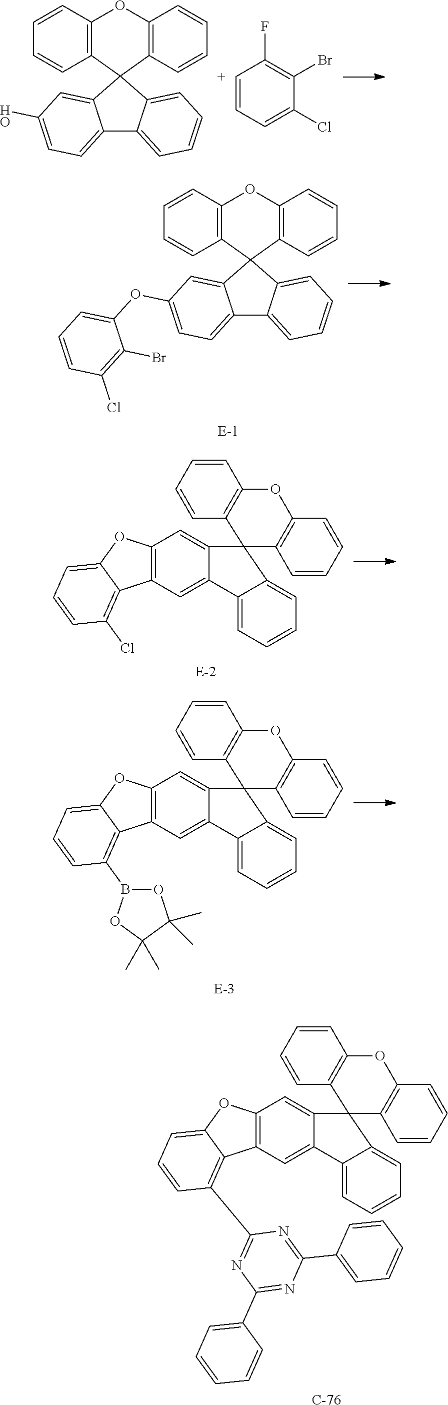

Synthesis of Compound E-1

Spiro[fluorene-9,9′-xanthen]-2-ol (90 g, 258 mmol), 2-bromo-1-chloro-3-fluorobenzene (54 g, 258 mmol), potassium carbonate (53 mg, 387 mmol), and 900 mL of NMP were added to a flask, and the mixture was refluxed at 160° C. for 4 hours. After completion of the reaction, an organic layer was extracted with ethyl acetate, and residual moisture was removed by using magnesium sulfate. The residue was dried and separated by column chromatography to obtain compound E-1 (97 g, yield: 70%).

Synthesis of Compound E-2

Compound E-1 (100 g, 186 mmol), Pd(OAc)₂ (2 g, 9.3 mmol), tricyclohexylphosphine (5.2 g, 18 mmol), potassium carbonate (77 g, 558 mmol), and 1.5 L of DMAc were added to a flask, and the mixture was reacted at 150° C. for 10 hours. After completion of the reaction, an organic layer was extracted with ethyl acetate, and residual moisture was removed by using magnesium sulfate. The residue was dried and separated by column chromatography to obtain compound E-2 (43 g, yield: 50%).

Synthesis of Compound E-3

Compound E-2 (43 g, 94 mmol), potassium acetate (27 g, 282 mmol), bis(pinacolato)diboron (31 g, 122 mmol), Pd(dba)₂ (1 g, 0.2% mol), tricyclohexylphosphine (1 g, 0.4 mol %), 18-crown-6 (0.5 g, 0.2 mol %), and 1.2 L of toluene were added to a flask, and the mixture was reacted at 110° C. for 8 hours. After completion of the reaction, an organic layer was extracted with ethyl acetate, and residual moisture was removed by using magnesium sulfate. The residue was dried and separated by column chromatography to obtain compound E-3 (39 g, yield: 75%).

Synthesis of Compound C-76

Compound E-3 (10 g, 18 mmol), 2-chloro-4,6-diphenyl-1,3,5-triazine (4.9 g, 18 mmol), Pd(PPh₃)₄ (0.4 g, 4% mol), potassium carbonate (9.8 g, 45 mmol), 100 mL of toluene, 25 mL of ethanol, and 25 mL of distilled water were added to a flask, and the mixture was reacted under reflux for 4 hours. After completion of the reaction, an organic layer was extracted with ethyl acetate and the residual moisture was removed by using magnesium sulfate. The residue was separated by column chromatography to obtain compound C-76 (5.1 g, yield: 43%).

| | MW | M.P. |
|---|---|---|
| C-76 | 653.73 | 300° C. |

Hereinafter, the properties of the organic electroluminescent device (OLED) according to one embodiment of the present disclosure will be explained. However, the following examples merely illustrate the properties of an OLED according to the present disclosure in detail, but the present disclosure is not limited to the following examples.

### Device Examples 1-1 to 1-5: Producing an OLED Comprising a First Host Compound and a Second Host Compound According to the Present Disclosure

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropanol. The ITO substrate was then mounted on a substrate holder of a vacuum vapor deposition apparatus. Compound H-1 shown in Table 4 below was introduced into a cell of the vacuum vapor deposition apparatus, and compound HT-1 shown in Table 4 below was introduced into another cell of the vacuum vapor deposition apparatus. The two materials were evaporated at different rates, and compound H-1 was deposited in a doping amount of 3 wt % based on the total amount of compound HI-1 and compound HT-1 to form a first hole injection layer having a thickness of 10 nm on the ITO substrate. Next, compound HT-1 was deposited on the first hole injection layer to form a first hole transport layer having a thickness of 80 nm. Compound HT-2 was then introduced into another cell of the vacuum vapor deposition apparatus and was evaporated by applying an electric current to the cell, thereby forming a second hole transport layer having a thickness of 30 nm on the first hole transport layer. After forming the hole injection layer and the hole transport layers, a light-emitting layer was formed thereon as follows: The first host compound and the second host compound shown in Table 1 below were introduced into two cells of the vacuum vapor depositing apparatus as hosts, and compound D-50 was introduced into another cell as a dopant. The two host materials were evaporated at a different rate of 2:1 and the dopant material was simultaneously evaporated at a different rate, and the dopant was deposited in a doping amount of 10 wt % based on the total amount of the hosts and the dopant to form a light-emitting layer having a thickness of 40 nm on the second hole transport layer. Compound ETL-1 and compound EIL-1 were evaporated in a weight ratio of 40:60 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, an OLED was produced. All the materials used for producing the OLED were purified by vacuum sublimation at 10⁻⁶ torr.

The light-emitting color and the time taken for luminance to decrease from 100% to 50% (lifetime; T50) at a luminance of 20,000 nit of the OLEDs produced in Device Examples 1-1 to 1-5 are provided in Table 1 below.

TABLE 1

| | | | Light- | |
|---|---|---|---|---|
| | First | Second | Emitting | Lifetime |
| | Host | Host | Color | (T50) [hr] |
| Device Example | C-11 | H-45 | Green | 281 |
| 1-1 | | | | |
| Device Example | C-12 | H-45 | Green | 404 |
| 1-2 | | | | |
| Device Example | C-131 | H-45 | Green | 866 |
| 1-3 | | | | |
| Device Example | C-76 | H-45 | Green | 344 |
| 1-4 | | | | |
| Device Example | C-1 | H-45 | Green | 514 |
| 1-5 | | | | |

### Device Example 2-1: Producing an OLED Comprising a Compound According to the Present Disclosure

An OLED was produced in the same manner as in Device Example 1-1, except that a light-emitting layer was formed as follows: compound C-131 was introduced into one cell of the vacuum vapor depositing apparatus as a host, and compound D-50 was introduced into another cell as a dopant. The two materials were evaporated at different rates to deposit the dopant in a doping amount of 10 wt % based on the total amount of the host and dopant, thereby forming a light-emitting layer having a thickness of 40 nm on the second hole transport layer.

### Comparative Example 2-1: Producing an OLED Comprising a Comparative Compound as a Host

An OLED was produced in the same manner as in Device Example 2-1, except that the compound shown in Table 2 below was used instead of compound C-131 as a host of a light-emitting layer.

The light-emitting color and the time taken for luminance to decrease from 100% to 50% (lifetime; T50) at a luminance of 20,000 nit of the OLEDs produced in Device Example 2-1 and Comparative Example 2-1 are provided in Table 2 below.

TABLE 2

| | | Light-Emitting | Lifetime |
|---|---|---|---|
| | Host | Color | (T50) [hr] |
| Device Example | C-131 | Green | 139 |
| 2-1 | | | |
| Comparative | C-3 | Green | 77 |
| Example 2-1 | | | |

### Device Examples 3-1 to 3-4: Producing an OLED Comprising a First Host Material and a Second Host Material According to the Present Disclosure

An OLED was produced in the same manner as in Device Example 1-1, except that the first host compound and the second host compound as shown in Table 3 below were used.

### Comparative Example 3-1: Producing an OLED Comprising a Comparative Compound as a Host

An OLED was produced in the same manner as in Device Example 3-1, except that the compound shown in Table 3 below was used as a first host of a light-emitting layer.

The light-emitting color and the time taken for luminance to decrease from 100% to 50% (lifetime; T50) at a luminance of 20,000 nit of the OLEDs produced in Device Examples 3-1 to 3-4 and Comparative Example 3-1 are provided in Table 3 below.

TABLE 3

| | | | Light- | |
|---|---|---|---|---|
| | First | Second | Emitting | Lifetime |
| | Host | Host | Color | (T50) [hr] |
| Device Example | C-11 | H2-2 | Green | 527 |
| 3-1 | | | | |
| Device Example | C-12 | H2-2 | Green | 786 |
| 3-2 | | | | |
| Device Example | C-131 | H2-2 | Green | 1674 |
| 3-3 | | | | |
| Device Example | C-76 | H2-2 | Green | 994 |
| 3-4 | | | | |
| Comparative | C-3 | H2-2 | Green | 358 |
| Example 3-1 | | | | |

From Table 2 above, it can be confirmed that the OLEDs comprising the compound according to the present disclosure as a host material exhibited longer lifetime properties compared to conventional organic electroluminescent devices. That is, it can be seen that organic electroluminescent compounds according to the present disclosure have better luminous properties than conventional materials. In addition, from Tables 1 and 3 above, it can be confirmed that the device using a specific combination of compounds according to the present disclosure as a plurality of host materials exhibited improved lifetime properties.

The compounds used in the Device Examples and the Comparative Examples are shown in Table 4 below.

TABLE 4

| | |
|---|---|
| Hole Injection Layer/ Hole Transport Layer | 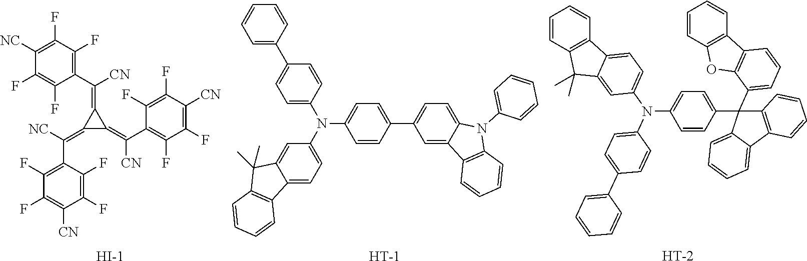 |
| Electron Transport Layer/ Electron Injection Layer | 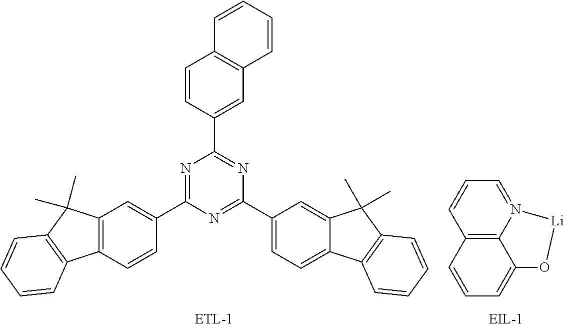 |

1. A plurality of host materials comprising a first host material comprising a compound represented by the following formula 1, and a second host material comprising a compound represented by the following formula 2, wherein the compound represented by the following formula 1 and the compound represented by the following formula 2 are different from each other: 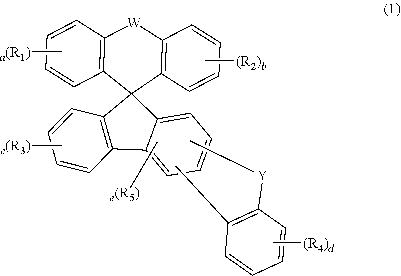
wherein
W represents a single bond, O, S, CR₆R₇, or N-L-R₈;
Y represents O, S, CR₆R₇, or N-L-R₈;
L, each independently, represents a single bond, a substituted or unsubstituted (C6-C30)arylene, or a substituted or unsubstituted (3- to 30-membered)heteroarylene;
R₁ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arysilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or adjacent ones of R₁ to R₈ may be linked to each other to form a ring(s); and
a to d, each independently, represent an integer of 1 to 4; and e represents an integer of 1 or 2; where each of a to e is an integer of 2 or more, each of R₁, each of R₂, each of R₃, each of R₄, and each of R₅ may be the same or different; 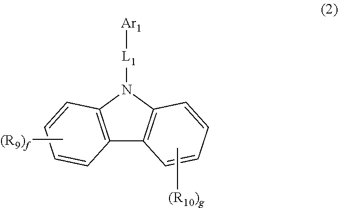
wherein
L₁ represents a single bond, or a substituted or unsubstituted (C6-C30)arylene;
Ar₁ represents a substituted or unsubstituted (C6-C30)aryl, or a substituted or unsubstituted nitrogen-containing (13- to 30-membered)heteroaryl;
R₉ and R₁₀, each independently, represent deuterium, a halogen, a cyano, a substituted or unsubstituted (C1-C30)alkyl, a substituted or unsubstituted (C6-C30)aryl, a substituted or unsubstituted (3- to 50-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or adjacent ones of R₉ and R₁₀ may be linked to each other to form a substituted or unsubstituted ring(s); with the proviso that if R₉ or R₁₀ represents a substituted (C6-C30)aryl, the substituent of the substituted (C6-C30)aryl is not a carbazolyl or a dibenzofuranyl; if R₉ or R₁₀ forms a substituted ring, the substituent of the substituted ring does not comprise a triazinyl; and if R₉ or R₁₀ represents a substituted or unsubstituted carbazolyl, R₉ or R₁₀ is linked to the backbone at a 9 position of the carbazolyl; and
f and g, each independently, represent an integer of 0 to 4, with the proviso that f and g are not zero at the same time; where each of f and g is an integer of 2 or more, each of R₉ and each of R₁₀ may be the same or different.

2. The plurality of host materials according to claim 1, wherein the formula 1 is represented by any one of the following formulas 1-1 to 1-4: 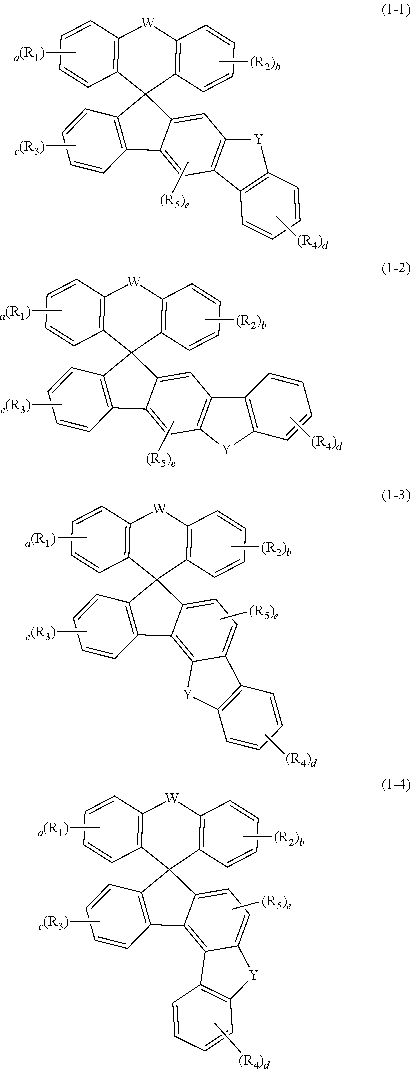
wherein W, Y, R₁ to R₅, and a to e are as defined in claim 1.

3. The plurality of host materials according to claim 1, wherein the formula 2 is represented by any one of the following formulas 2-1 to 2-3. 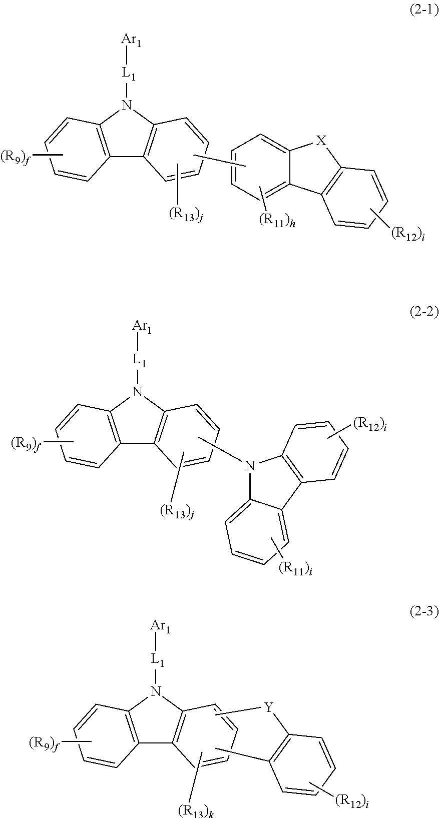
wherein,
X represents O, S, or CR₁₄CR₁₅;
Y represents N-L₂-Ar₂, O, S, or CR₁₄CR₁₅;
L₂ represents a single bond, or a substituted or unsubstituted (C6-C30)arylene;
Ar₂ represents a substituted or unsubstituted (C6-C30)aryl;
R₁₁ to R₁₅, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arysilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or two or more adjacent R₁₁'s may be linked to each other to form a ring(s), two or more adjacent R₁₂'s may be linked to each other to form a ring(s), two or more adjacent R₁₃'s may be linked to each other to form a ring(s), and R₁₄ and R₁₅ may be linked to each other to form a ring(s);
with the proviso that, in formula 2-3, R₁₂ does not comprise a triazinyl;
i and l, each independently, represent an integer of 1 to 4; h and j, each independently, represent an integer of 1 to 3; and k represents an integer of 1 or 2; where each of h to l is an integer of 2 or more, each of R₁₁, each of R₁₂, and each of R₁₃ may be the same or different; and
L₁, Ar₁, R₉ and f are as defined in claim 1.

4. The plurality of host materials according to claim 1, wherein the substituents of the substituted alkyl, the substituted aryl(ene), the substituted heteroaryl(ene), the substituted trialkylsilyl, the substituted dialkylarylsilyl, the substituted alkyldiarylsilyl, the substituted triarylsilyl, the substituted mono- or di-alkylamino, the substituted alkylarylamino, the substituted mono- or di-arylamino, and the substituted ring(s), each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; a (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with a (3- to 30-membered)heteroaryl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; an amino; a mono- or di-(C1-C30)alkylamino; a mono- or di-(C2-C30)alkenylamino; a mono- or di-(C6-C30)arylamino unsubstituted or substituted with a (C1-C30)alkyl(s); a mono- or di-(3- to 30-membered)heteroarylamino; a (C1-C30)alkyl(C2-C30)alkenylamino; a (C1-C30)alkyl(C6-C30)arylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

5. The plurality of host materials according to claim 1, wherein the compound represented by formula 1 is at least one selected from the following compounds:

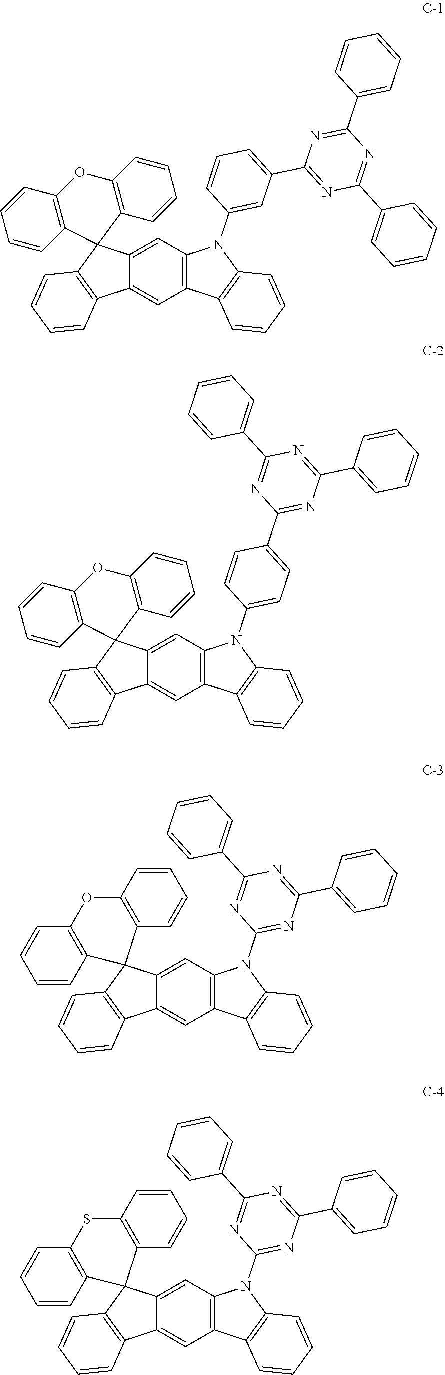 [SMILES: c1ccc(-c2nc(-c3ccccc3)nc(-c3cccc(-n4c5ccccc5c5cc6c(cc54)C4(c5ccccc5Oc5ccccc54)c4ccccc4-6)c3)n2)cc1 ; c1ccc(-c2nc(-c3ccccc3)nc(-c3ccc(-n4c5ccccc5c5cc6c(cc54)C4(c5ccccc5Oc5ccccc54)c4ccccc4-6)cc3)n2)cc1 ; c1ccc(-c2nc(-c3ccccc3)nc(-n3c4ccccc4c4cc5c(cc43)C3(c4ccccc4Oc4ccccc43)c3ccccc3-5)n2)cc1 ; c1ccc(-c2nc(-c3ccccc3)nc(-n3c4ccccc4c4cc5c(cc43)C3(c4ccccc4Sc4ccccc43)c3ccccc3-5)n2)cc1]

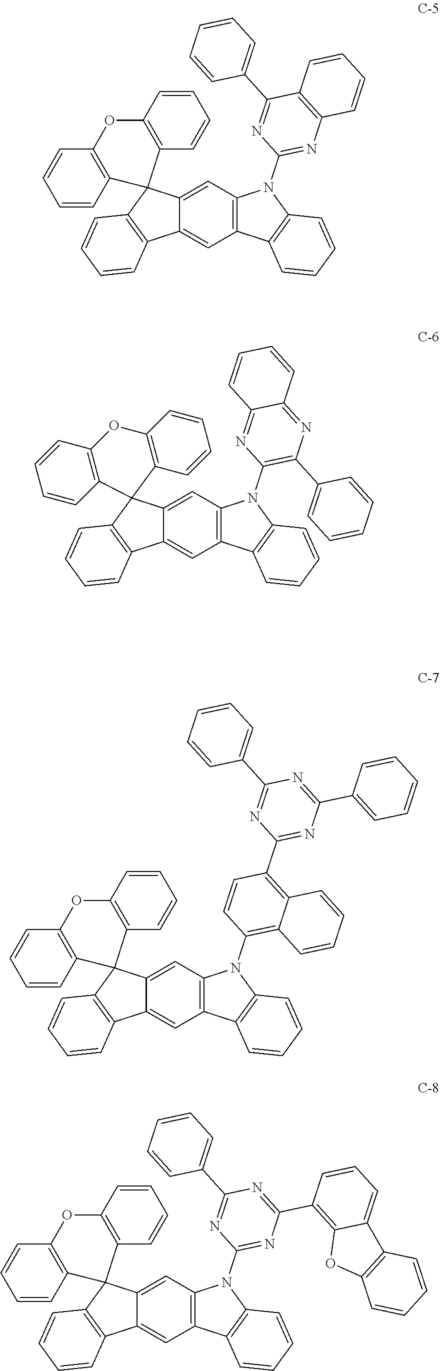 [SMILES: c1ccc(-c2nc(-n3c4ccccc4c4cc5c(cc43)C3(c4ccccc4Oc4ccccc43)c3ccccc3-5)nc3ccccc23)cc1 ; c1ccc(-c2nc3ccccc3nc2-n2c3ccccc3c3cc4c(cc32)C2(c3ccccc3Oc3ccccc32)c2ccccc2-4)cc1 ; c1ccc(-c2nc(-c3ccccc3)nc(-c3ccc(-n4c5ccccc5c5cc6c(cc54)C4(c5ccccc5Oc5ccccc54)c4ccccc4-6)c4ccccc34)n2)cc1 ; c1ccc(-c2nc(-c3cccc4c3oc3ccccc34)nc(-n3c4ccccc4c4cc5c(cc43)C3(c4ccccc4Oc4ccccc43)c3ccccc3-5)n2)cc1]

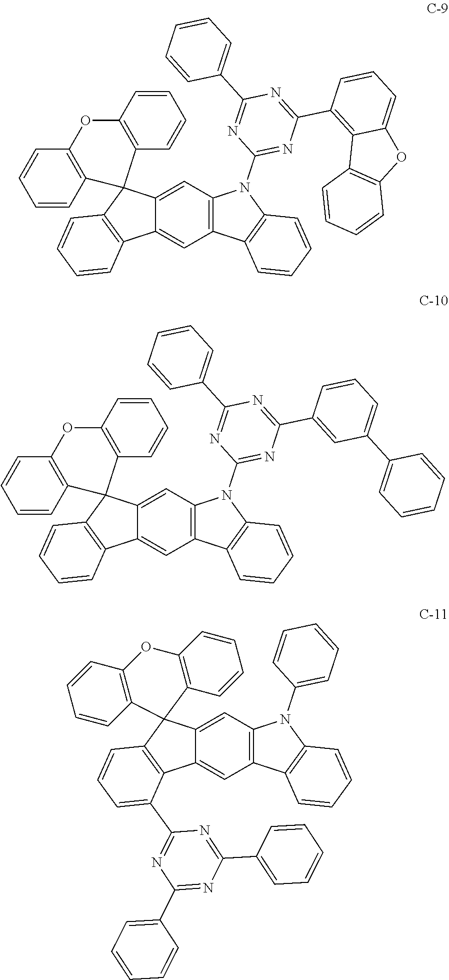 [SMILES: c1ccc(-c2nc(-c3cccc4oc5ccccc5c34)nc(-n3c4ccccc4c4cc5c(cc43)C3(c4ccccc4Oc4ccccc43)c3ccccc3-5)n2)cc1 ; c1ccc(-c2cccc(-c3nc(-c4ccccc4)nc(-n4c5ccccc5c5cc6c(cc54)C4(c5ccccc5Oc5ccccc54)c4ccccc4-6)n3)c2)cc1 ; c1ccc(-c2nc(-c3ccccc3)nc(-c3cccc4c3-c3cc5c6ccccc6n(-c6ccccc6)c5cc3C43c4ccccc4Oc4ccccc43)n2)cc1]

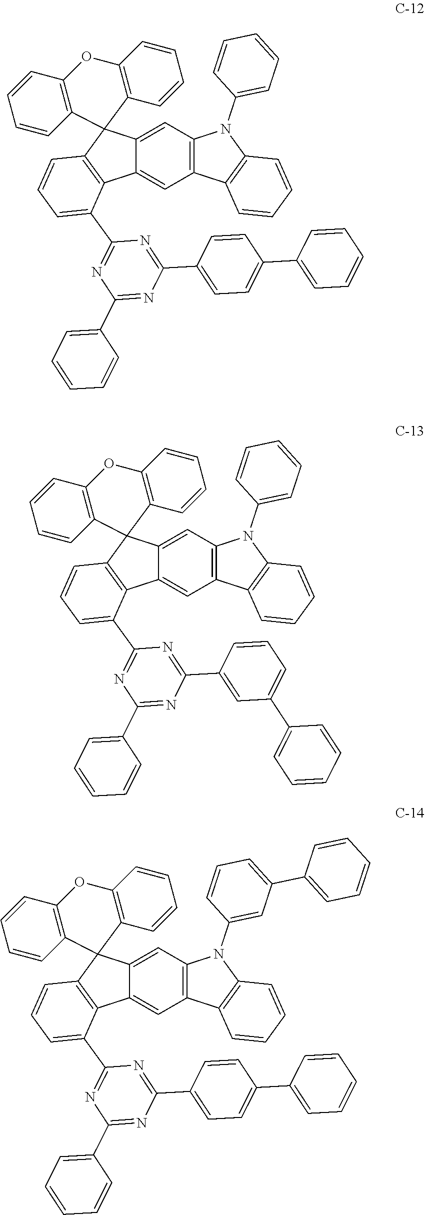 [SMILES: c1ccc(-c2ccc(-c3nc(-c4ccccc4)nc(-c4cccc5c4-c4cc6c7ccccc7n(-c7ccccc7)c6cc4C54c5ccccc5Oc5ccccc54)n3)cc2)cc1 ; c1ccc(-c2cccc(-c3nc(-c4ccccc4)nc(-c4cccc5c4-c4cc6c7ccccc7n(-c7ccccc7)c6cc4C54c5ccccc5Oc5ccccc54)n3)c2)cc1 ; c1ccc(-c2ccc(-c3nc(-c4ccccc4)nc(-c4cccc5c4-c4cc6c7ccccc7n(-c7cccc(-c8ccccc8)c7)c6cc4C54c5ccccc5Oc5ccccc54)n3)cc2)cc1]

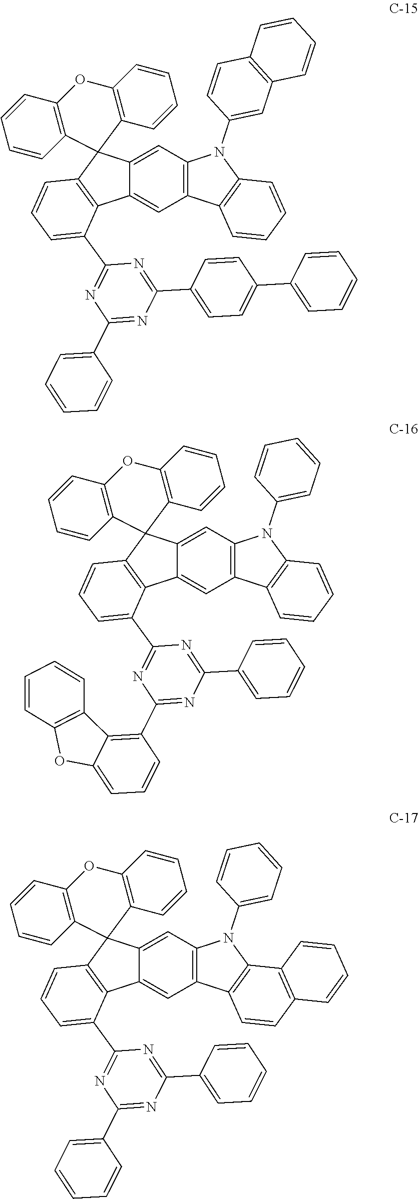 [SMILES: c1ccc(-c2ccc(-c3nc(-c4ccccc4)nc(-c4cccc5c4-c4cc6c7ccccc7n(-c7ccc8ccccc8c7)c6cc4C54c5ccccc5Oc5ccccc54)n3)cc2)cc1 ; c1ccc(-c2nc(-c3cccc4c3-c3cc5c6ccccc6n(-c6ccccc6)c5cc3C43c4ccccc4Oc4ccccc43)nc(-c3cccc4oc5ccccc5c34)n2)cc1 ; c1ccc(-c2nc(-c3ccccc3)nc(-c3cccc4c3-c3cc5c6ccc7ccccc7c6n(-c6ccccc6)c5cc3C43c4ccccc4Oc4ccccc43)n2)cc1]

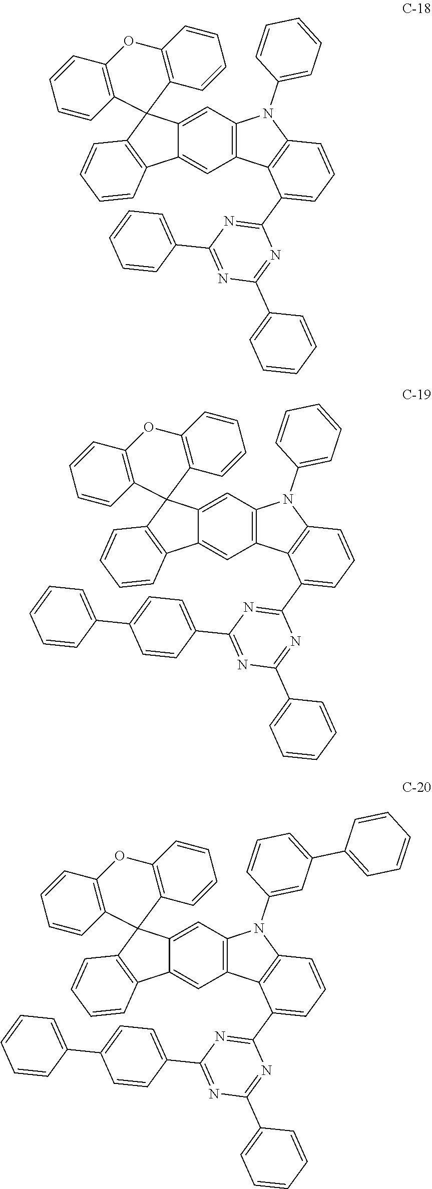

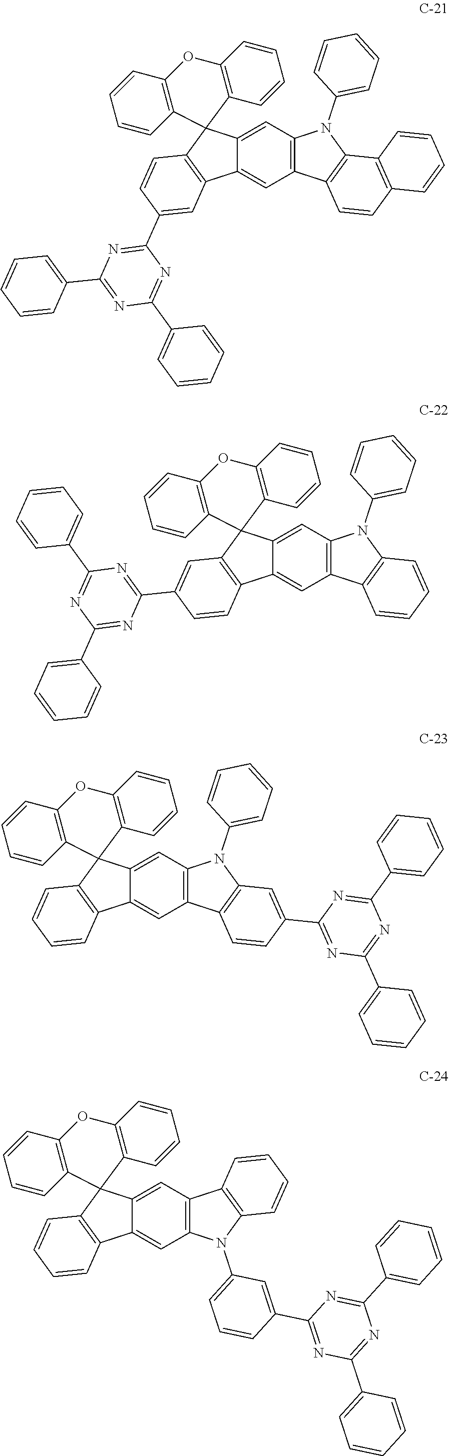 [SMILES: c1ccc(-c2nc(-c3ccccc3)nc(-c3ccc4c(c3)-c3cc5c6ccc7ccccc7c6n(-c6ccccc6)c5cc3C43c4ccccc4Oc4ccccc43)n2)cc1 ; c1ccc(-c2nc(-c3ccccc3)nc(-c3ccc4c(c3)C3(c5ccccc5Oc5ccccc53)c3cc5c(cc3-4)c3ccccc3n5-c3ccccc3)n2)cc1 ; c1ccc(-c2nc(-c3ccccc3)nc(-c3ccc4c5cc6c(cc5n(-c5ccccc5)c4c3)C3(c4ccccc4Oc4ccccc43)c3ccccc3-6)n2)cc1 ; c1ccc(-c2nc(-c3ccccc3)nc(-c3cccc(-n4c5ccccc5c5cc6c(cc54)-c4ccccc4C64c5ccccc5Oc5ccccc54)c3)n2)cc1]

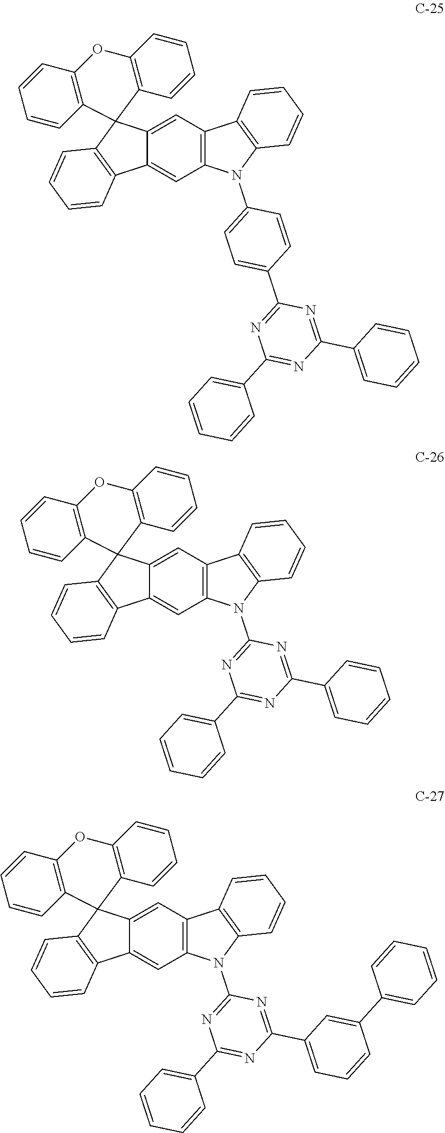

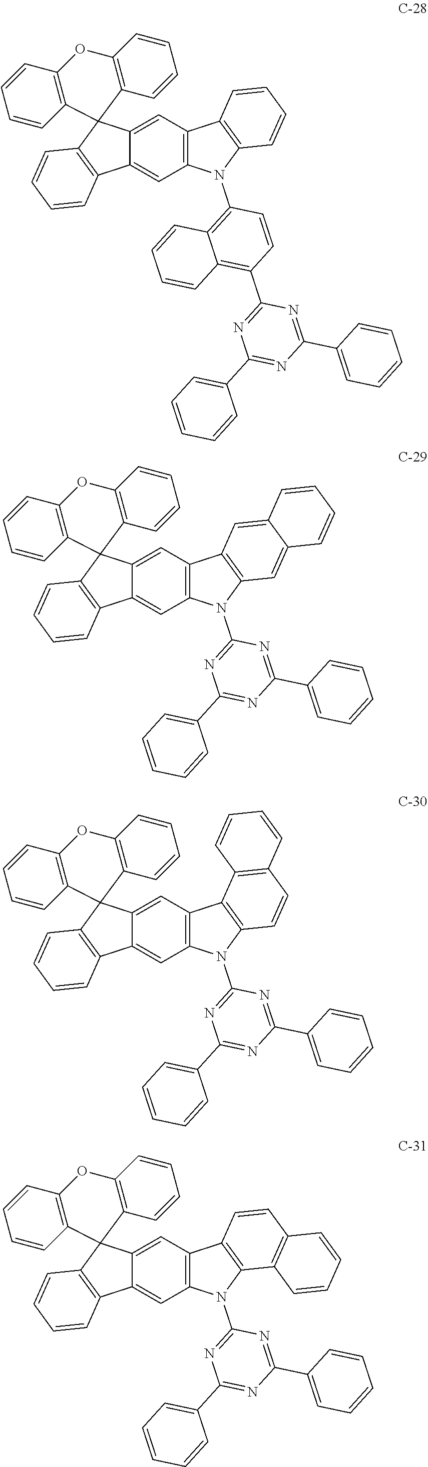

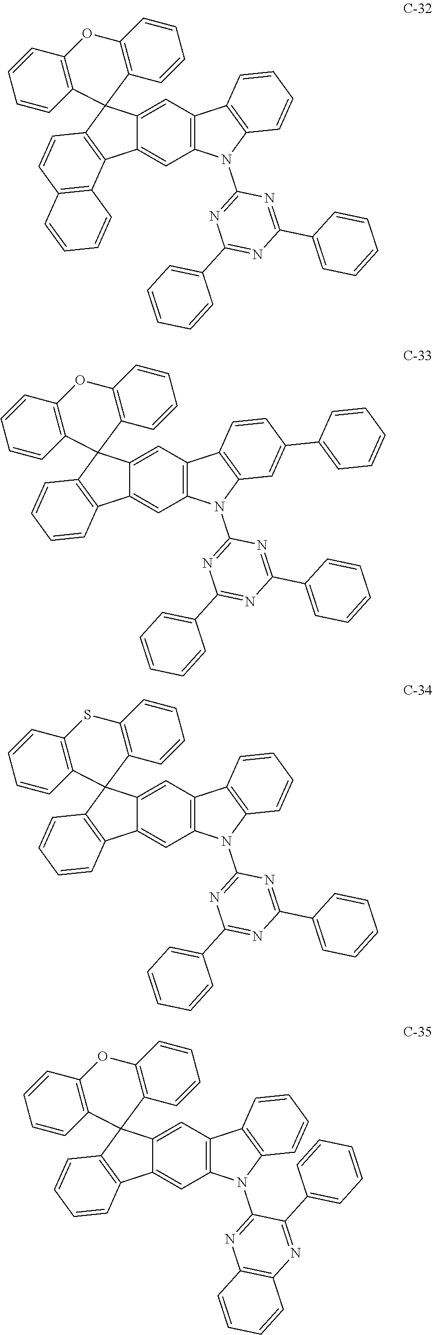

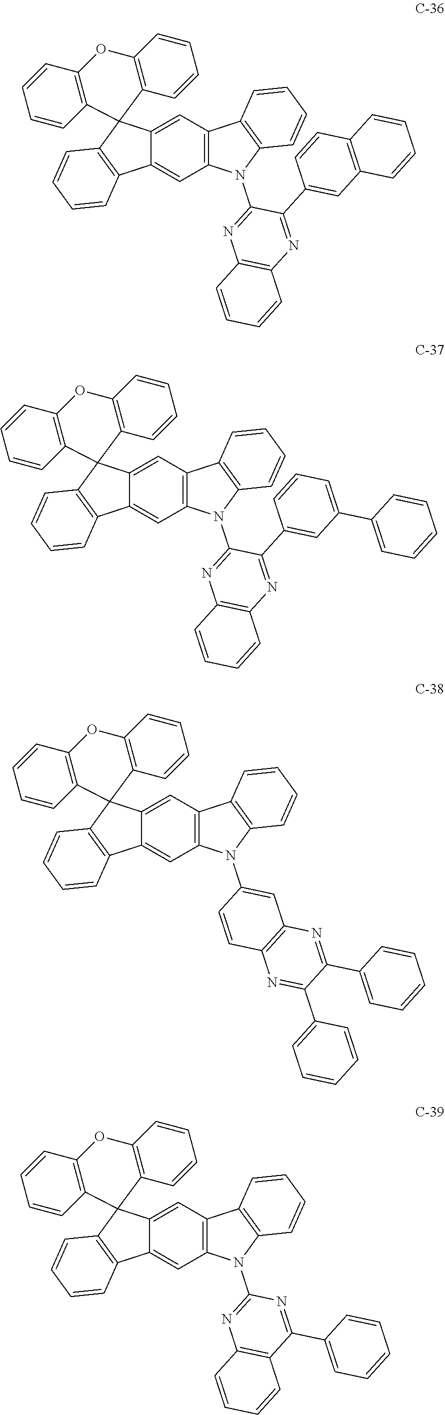 [SMILES: c1ccc2c(c1)Oc1ccccc1C21c2ccccc2-c2cc3c(cc21)c1ccccc1n3-c1nc2ccccc2nc1-c1ccc2ccccc2c1 ; c1ccc(-c2cccc(-c3nc4ccccc4nc3-n3c4ccccc4c4cc5c(cc43)-c3ccccc3C53c4ccccc4Oc4ccccc43)c2)cc1 ; c1ccc(-c2nc3ccc(-n4c5ccccc5c5cc6c(cc54)-c4ccccc4C64c5ccccc5Oc5ccccc54)cc3nc2-c2ccccc2)cc1 ; c1ccc(-c2nc(-n3c4ccccc4c4cc5c(cc43)-c3ccccc3C53c4ccccc4Oc4ccccc43)nc3ccccc23)cc1]

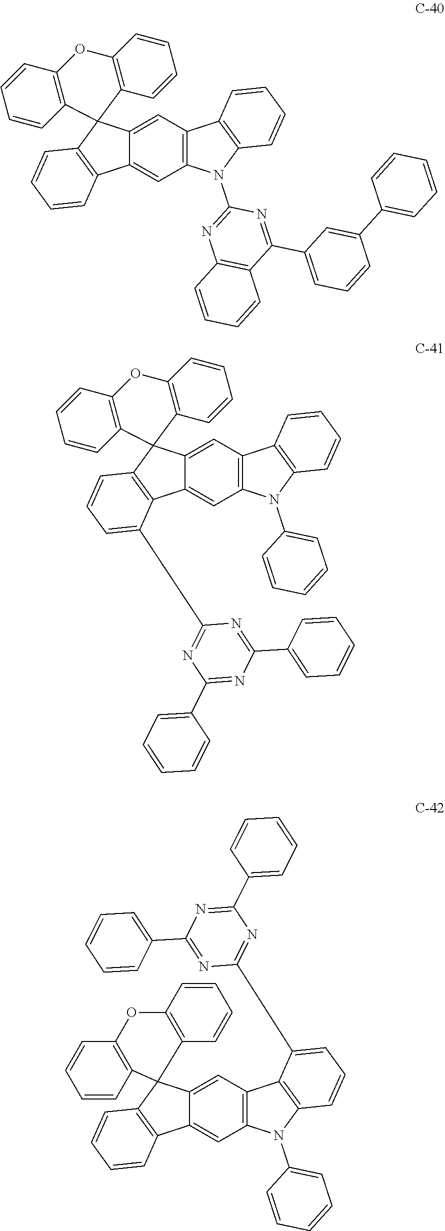

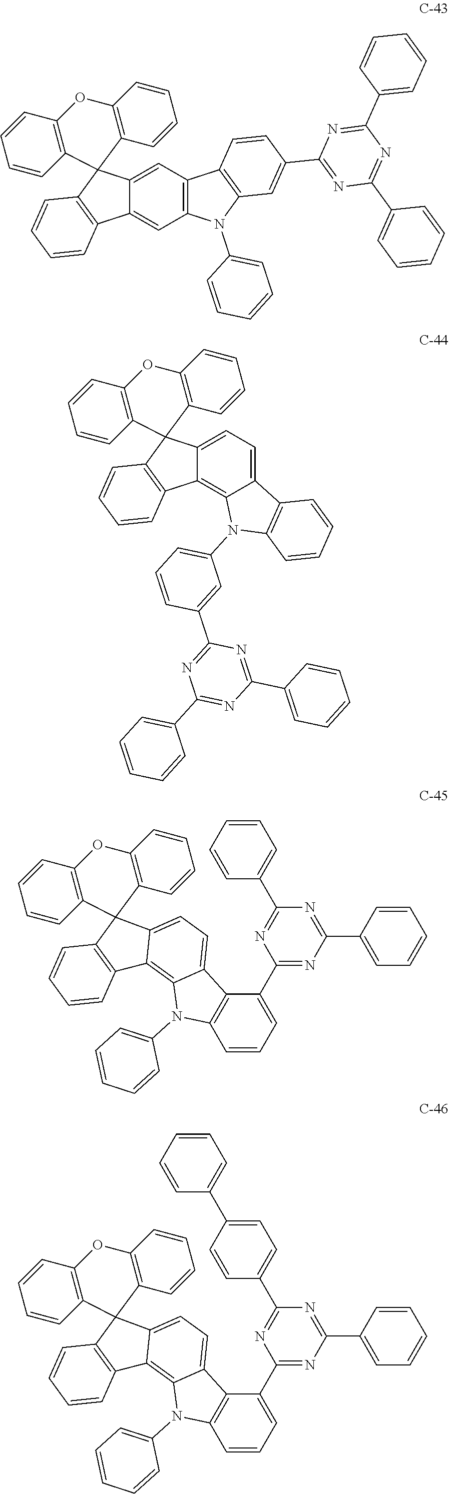

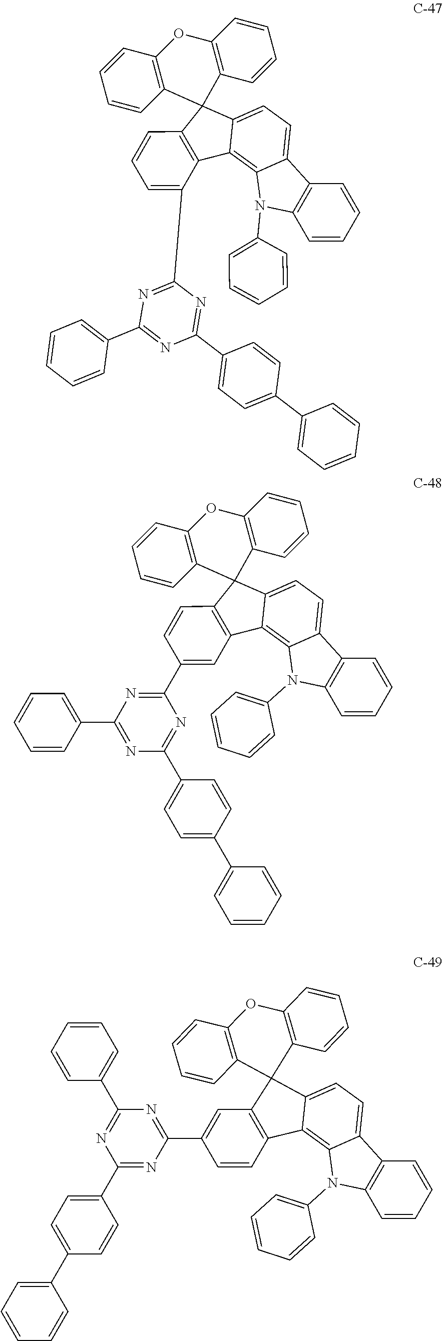

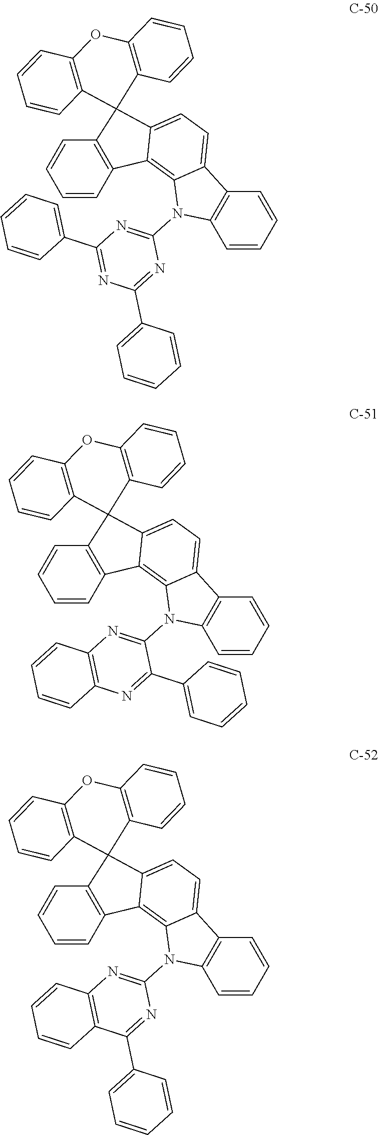

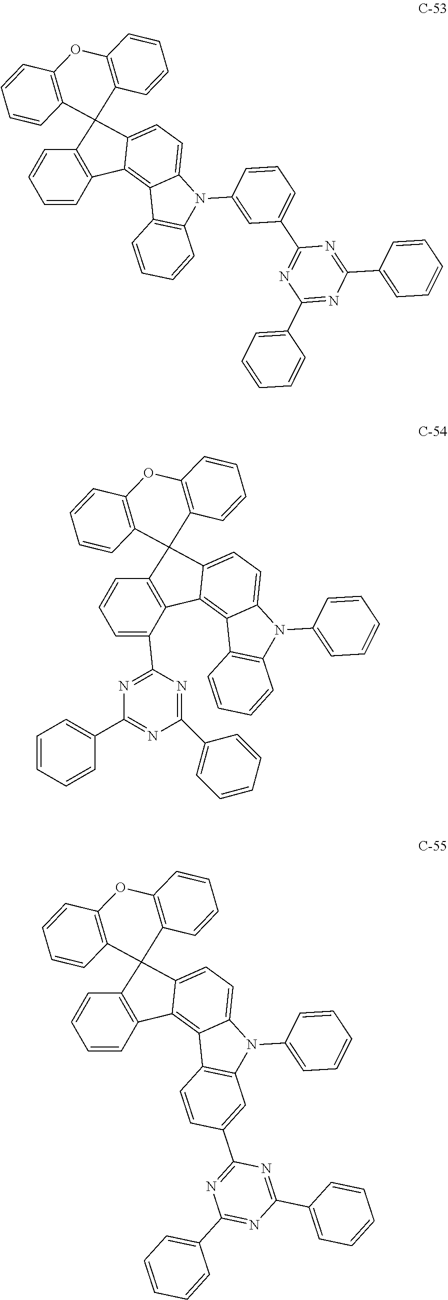 [SMILES: c1ccc(-c2nc(-c3ccccc3)nc(-c3cccc(-n4c5ccccc5c5c6c(ccc54)C4(c5ccccc5Oc5ccccc54)c4ccccc4-6)c3)n2)cc1 ; c1ccc(-c2nc(-c3ccccc3)nc(-c3cccc4c3-c3c(ccc5c3c3ccccc3n5-c3ccccc3)C43c4ccccc4Oc4ccccc43)n2)cc1 ; c1ccc(-c2nc(-c3ccccc3)nc(-c3ccc4c5c6c(ccc5n(-c5ccccc5)c4c3)C3(c4ccccc4Oc4ccccc43)c3ccccc3-6)n2)cc1]

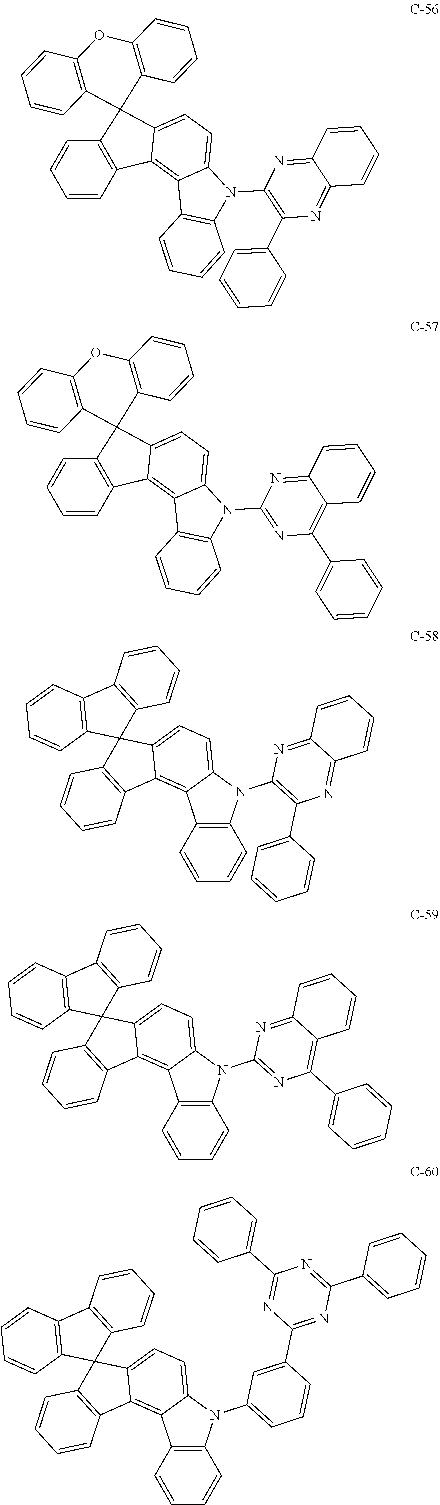

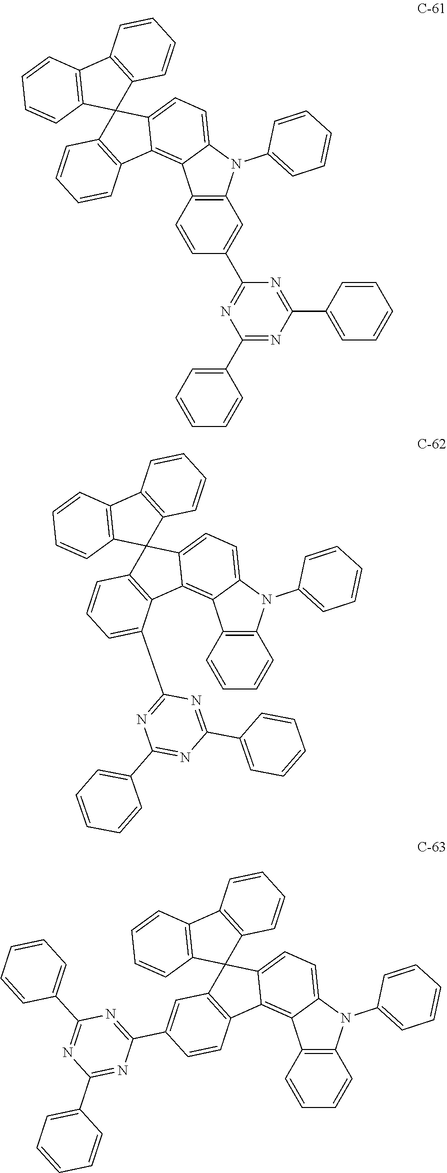

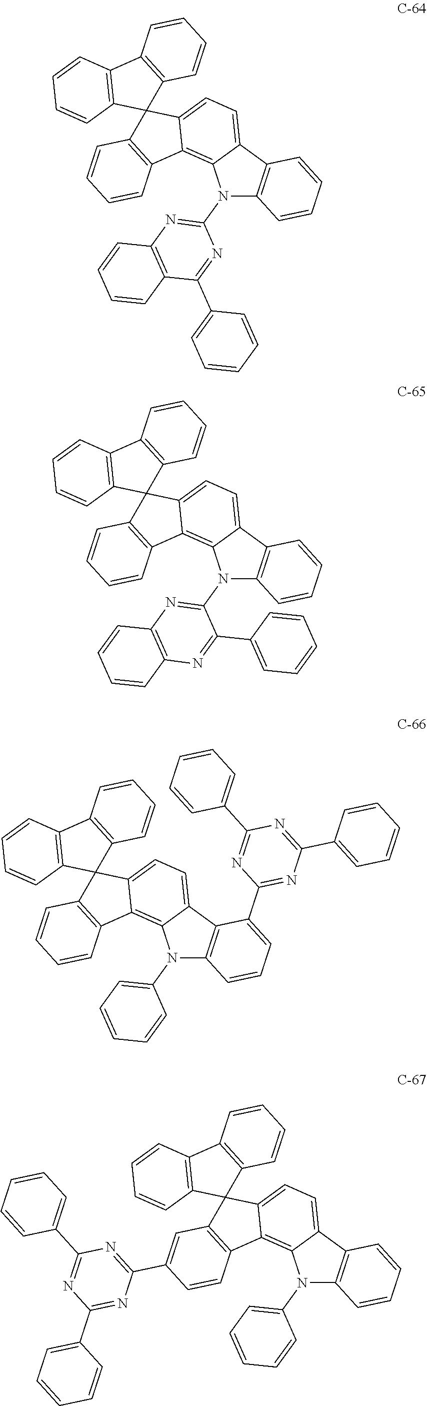

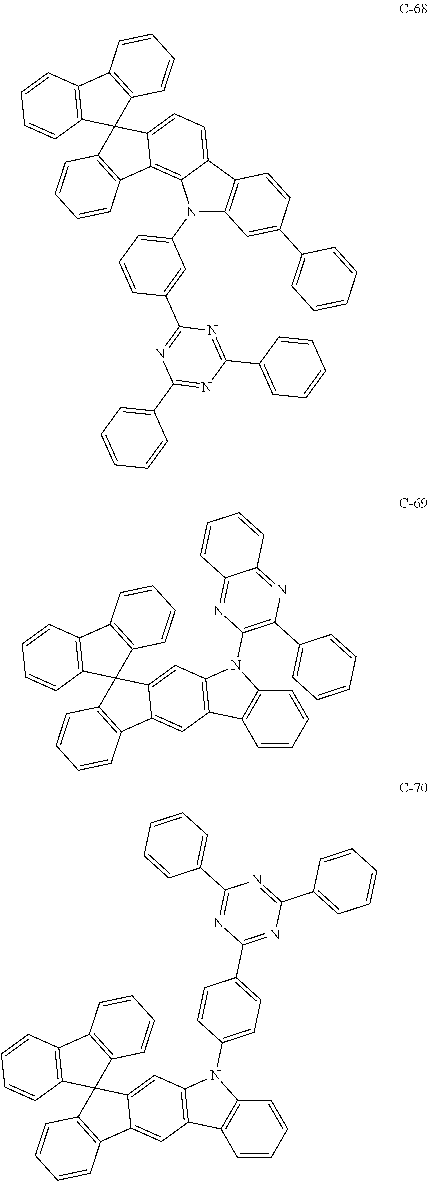 [SMILES: c1ccc(-c2ccc3c4ccc5c(c4n(-c4cccc(-c6nc(-c7ccccc7)nc(-c7ccccc7)n6)c4)c3c2)-c2ccccc2C52c3ccccc3-c3ccccc32)cc1 ; c1ccc(-c2nc3ccccc3nc2-n2c3ccccc3c3cc4c(cc32)C2(c3ccccc3-c3ccccc32)c2ccccc2-4)cc1 ; c1ccc(-c2nc(-c3ccccc3)nc(-c3ccc(-n4c5ccccc5c5cc6c(cc54)C4(c5ccccc5-c5ccccc54)c4ccccc4-6)cc3)n2)cc1]

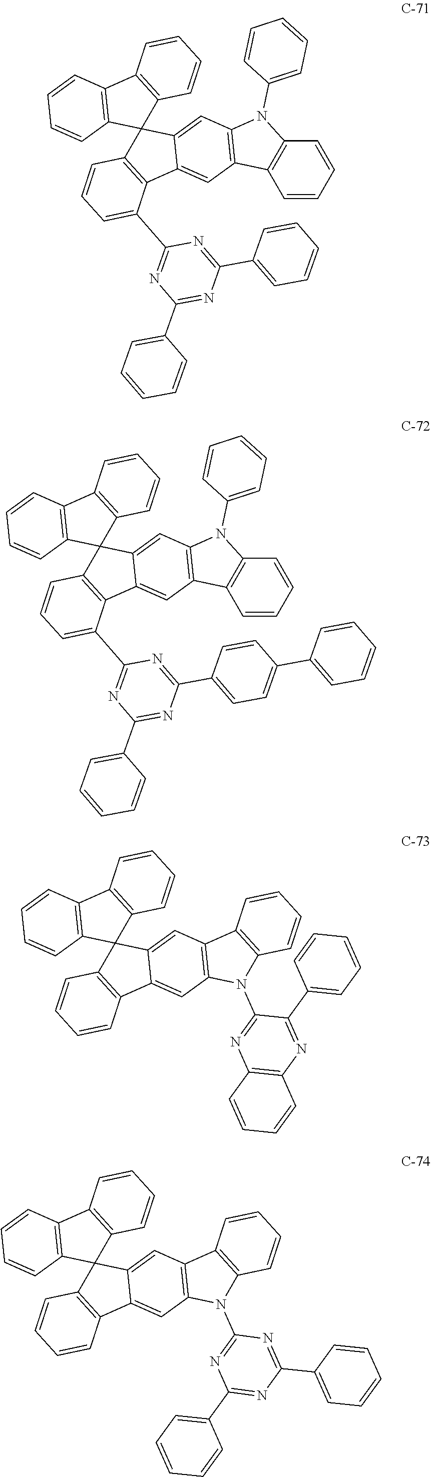

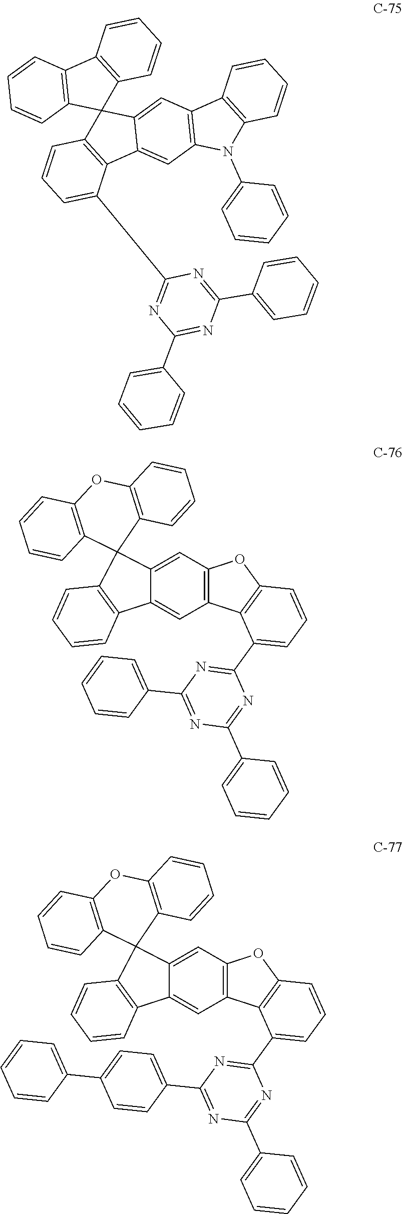

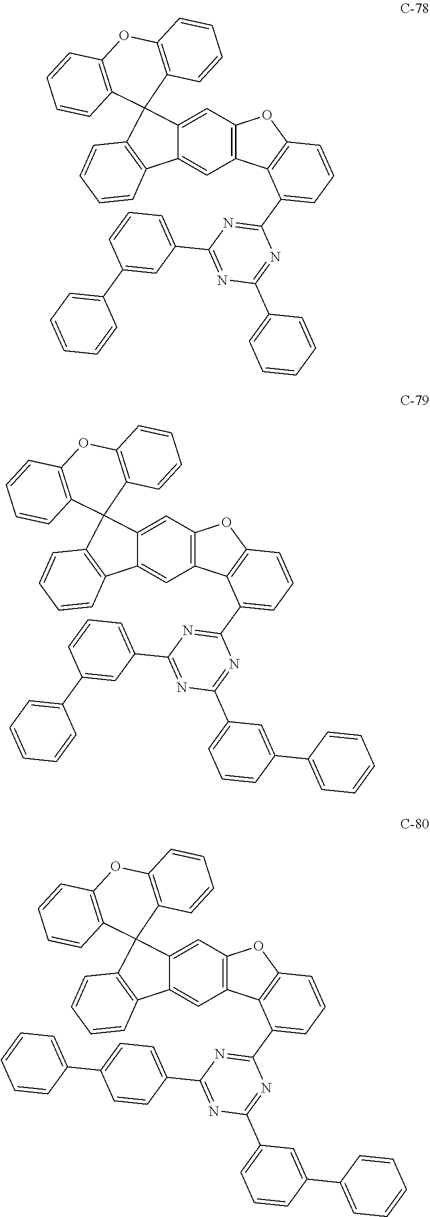 [SMILES: c1ccc(-c2cccc(-c3nc(-c4ccccc4)nc(-c4cccc5oc6cc7c(cc6c45)-c4ccccc4C74c5ccccc5Oc5ccccc54)n3)c2)cc1 ; c1ccc(-c2cccc(-c3nc(-c4cccc(-c5ccccc5)c4)nc(-c4cccc5oc6cc7c(cc6c45)-c4ccccc4C74c5ccccc5Oc5ccccc54)n3)c2)cc1 ; c1ccc(-c2ccc(-c3nc(-c4cccc(-c5ccccc5)c4)nc(-c4cccc5oc6cc7c(cc6c45)-c4ccccc4C74c5ccccc5Oc5ccccc54)n3)cc2)cc1]

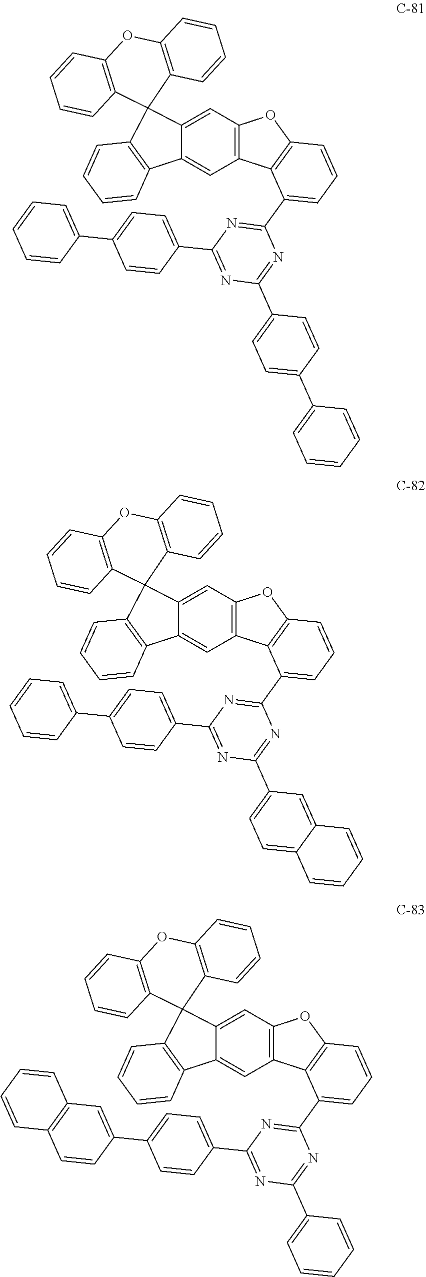 [SMILES: c1ccc(-c2ccc(-c3nc(-c4ccc(-c5ccccc5)cc4)nc(-c4cccc5oc6cc7c(cc6c45)-c4ccccc4C74c5ccccc5Oc5ccccc54)n3)cc2)cc1 ; c1ccc(-c2ccc(-c3nc(-c4ccc5ccccc5c4)nc(-c4cccc5oc6cc7c(cc6c45)-c4ccccc4C74c5ccccc5Oc5ccccc54)n3)cc2)cc1 ; c1ccc(-c2nc(-c3ccc(-c4ccc5ccccc5c4)cc3)nc(-c3cccc4oc5cc6c(cc5c34)-c3ccccc3C63c4ccccc4Oc4ccccc43)n2)cc1]

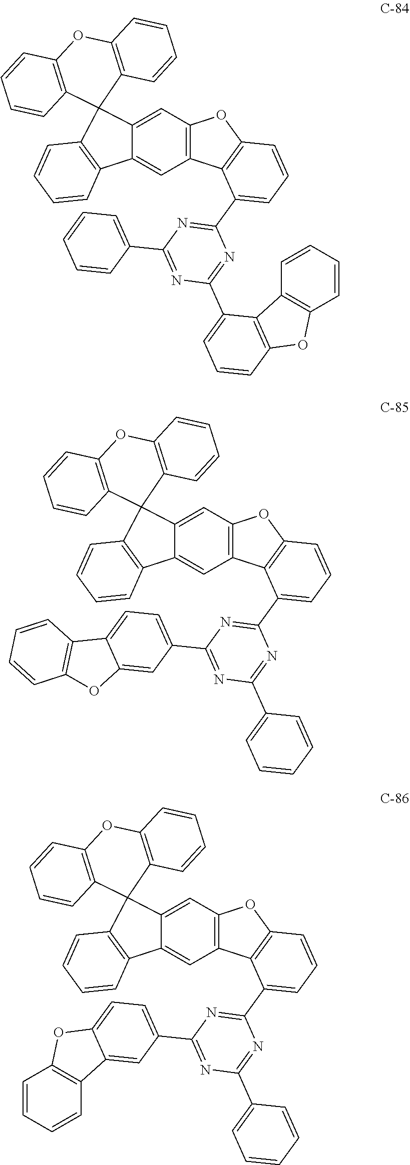 [SMILES: c1ccc(-c2nc(-c3cccc4oc5ccccc5c34)nc(-c3cccc4oc5cc6c(cc5c34)-c3ccccc3C63c4ccccc4Oc4ccccc43)n2)cc1 ; c1ccc(-c2nc(-c3ccc4c(c3)oc3ccccc34)nc(-c3cccc4oc5cc6c(cc5c34)-c3ccccc3C63c4ccccc4Oc4ccccc43)n2)cc1 ; c1ccc(-c2nc(-c3ccc4oc5ccccc5c4c3)nc(-c3cccc4oc5cc6c(cc5c34)-c3ccccc3C63c4ccccc4Oc4ccccc43)n2)cc1]

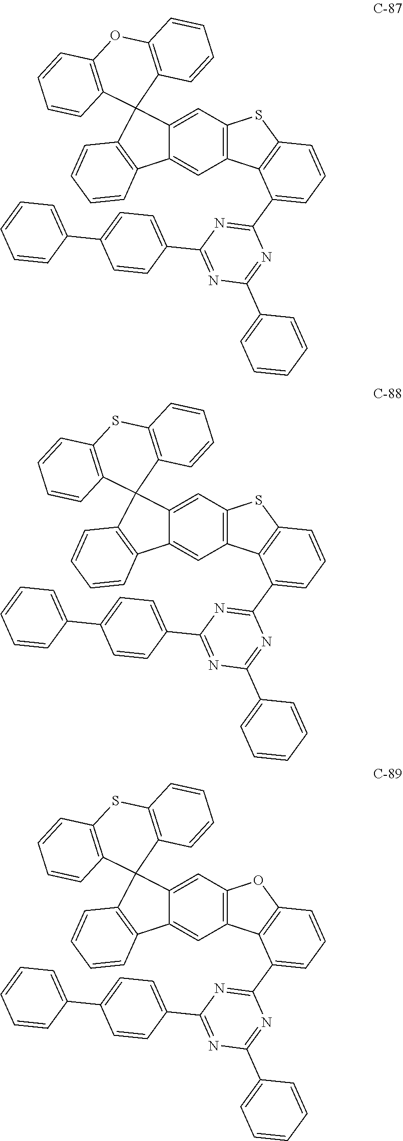 [SMILES: c1ccc(-c2ccc(-c3nc(-c4ccccc4)nc(-c4cccc5sc6cc7c(cc6c45)-c4ccccc4C74c5ccccc5Oc5ccccc54)n3)cc2)cc1 ; c1ccc(-c2ccc(-c3nc(-c4ccccc4)nc(-c4cccc5sc6cc7c(cc6c45)-c4ccccc4C74c5ccccc5Sc5ccccc54)n3)cc2)cc1 ; c1ccc(-c2ccc(-c3nc(-c4ccccc4)nc(-c4cccc5oc6cc7c(cc6c45)-c4ccccc4C74c5ccccc5Sc5ccccc54)n3)cc2)cc1]

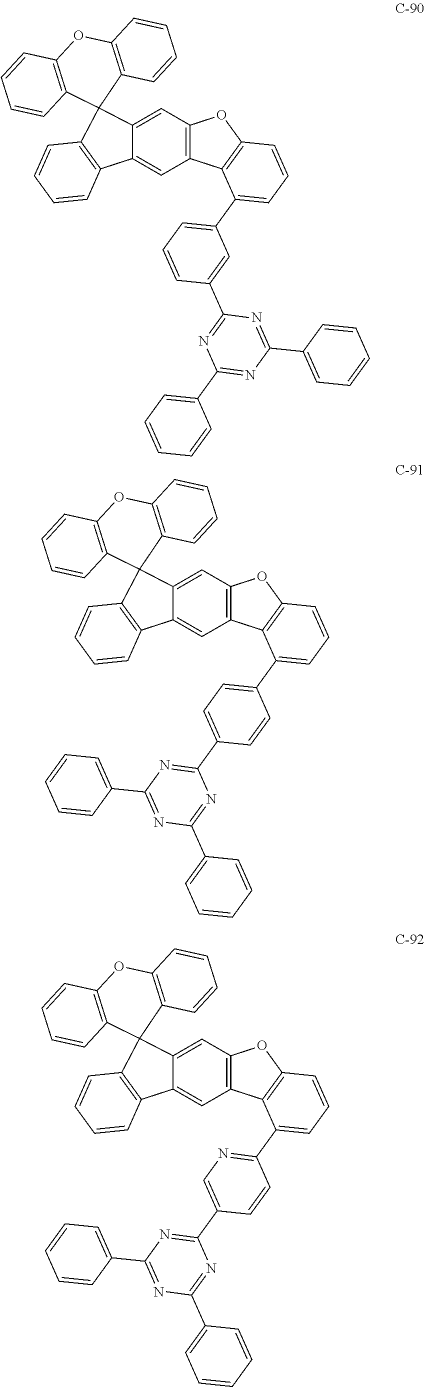

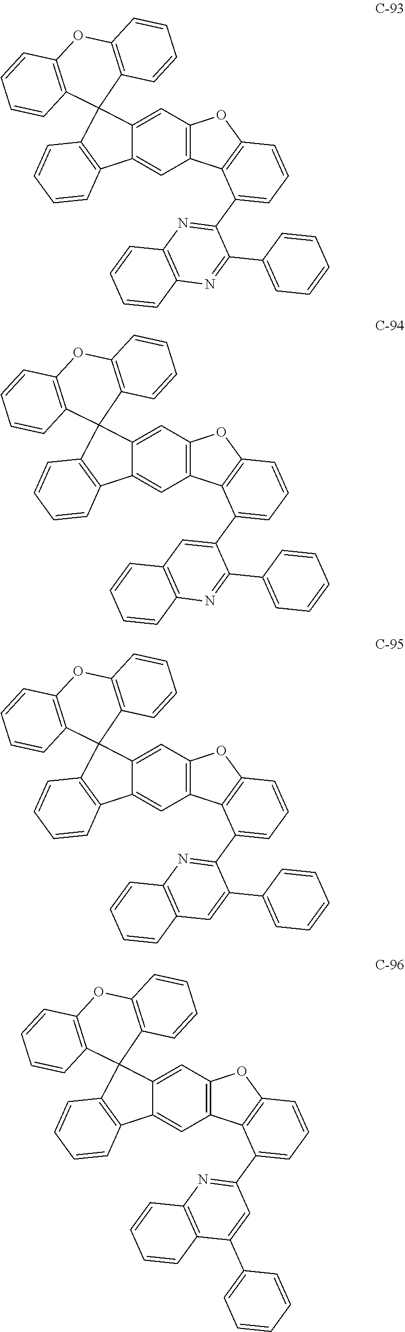

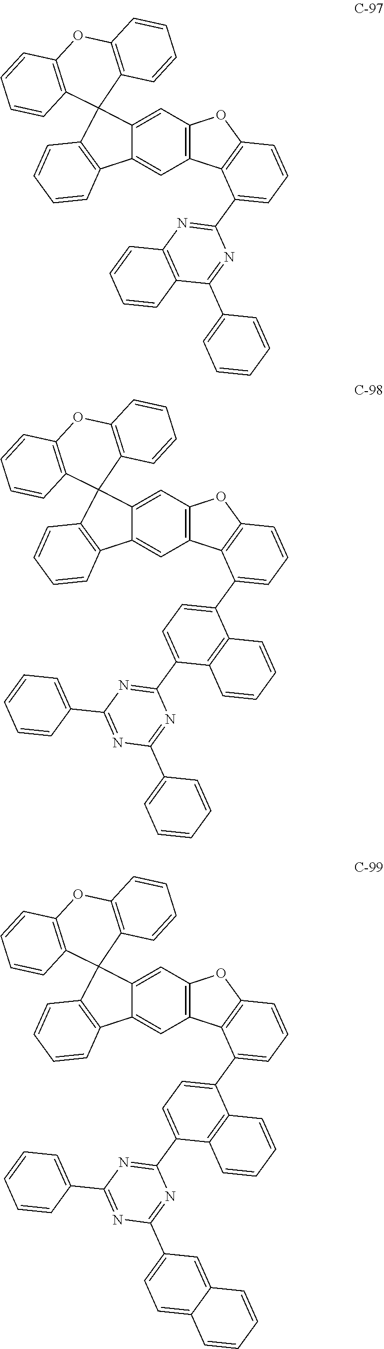 [SMILES: c1ccc(-c2nc(-c3cccc4oc5cc6c(cc5c34)-c3ccccc3C63c4ccccc4Oc4ccccc43)nc3ccccc23)cc1 ; c1ccc(-c2nc(-c3ccccc3)nc(-c3ccc(-c4cccc5oc6cc7c(cc6c45)-c4ccccc4C74c5ccccc5Oc5ccccc54)c4ccccc34)n2)cc1 ; c1ccc(-c2nc(-c3ccc4ccccc4c3)nc(-c3ccc(-c4cccc5oc6cc7c(cc6c45)-c4ccccc4C74c5ccccc5Oc5ccccc54)c4ccccc34)n2)cc1]

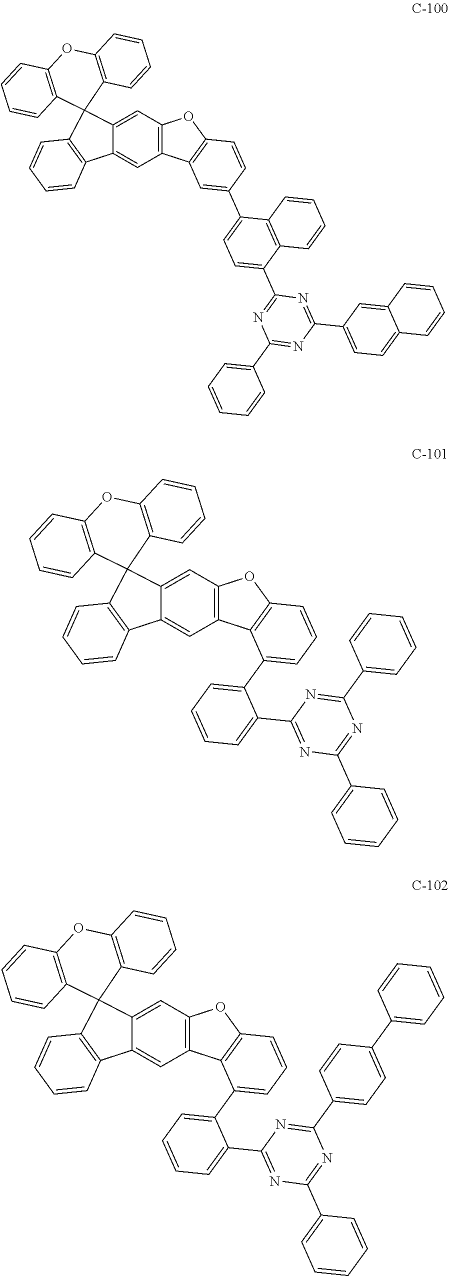 [SMILES: c1ccc(-c2nc(-c3ccc4ccccc4c3)nc(-c3ccc(-c4ccc5oc6cc7c(cc6c5c4)-c4ccccc4C74c5ccccc5Oc5ccccc54)c4ccccc34)n2)cc1 ; c1ccc(-c2nc(-c3ccccc3)nc(-c3ccccc3-c3cccc4oc5cc6c(cc5c34)-c3ccccc3C63c4ccccc4Oc4ccccc43)n2)cc1 ; c1ccc(-c2ccc(-c3nc(-c4ccccc4)nc(-c4ccccc4-c4cccc5oc6cc7c(cc6c45)-c4ccccc4C74c5ccccc5Oc5ccccc54)n3)cc2)cc1]

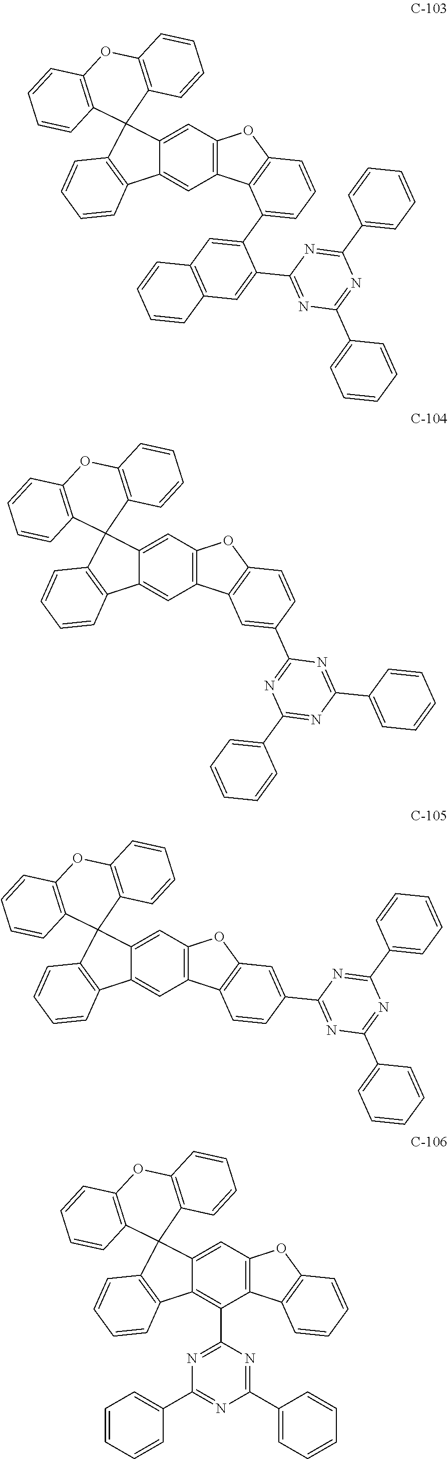 [SMILES: c1ccc(-c2nc(-c3ccccc3)nc(-c3cc4ccccc4cc3-c3cccc4oc5cc6c(cc5c34)-c3ccccc3C63c4ccccc4Oc4ccccc43)n2)cc1 ; c1ccc(-c2nc(-c3ccccc3)nc(-c3ccc4oc5cc6c(cc5c4c3)-c3ccccc3C63c4ccccc4Oc4ccccc43)n2)cc1 ; c1ccc(-c2nc(-c3ccccc3)nc(-c3ccc4c(c3)oc3cc5c(cc34)-c3ccccc3C53c4ccccc4Oc4ccccc43)n2)cc1 ; c1ccc(-c2nc(-c3ccccc3)nc(-c3c4c(cc5oc6ccccc6c35)C3(c5ccccc5Oc5ccccc53)c3ccccc3-4)n2)cc1]

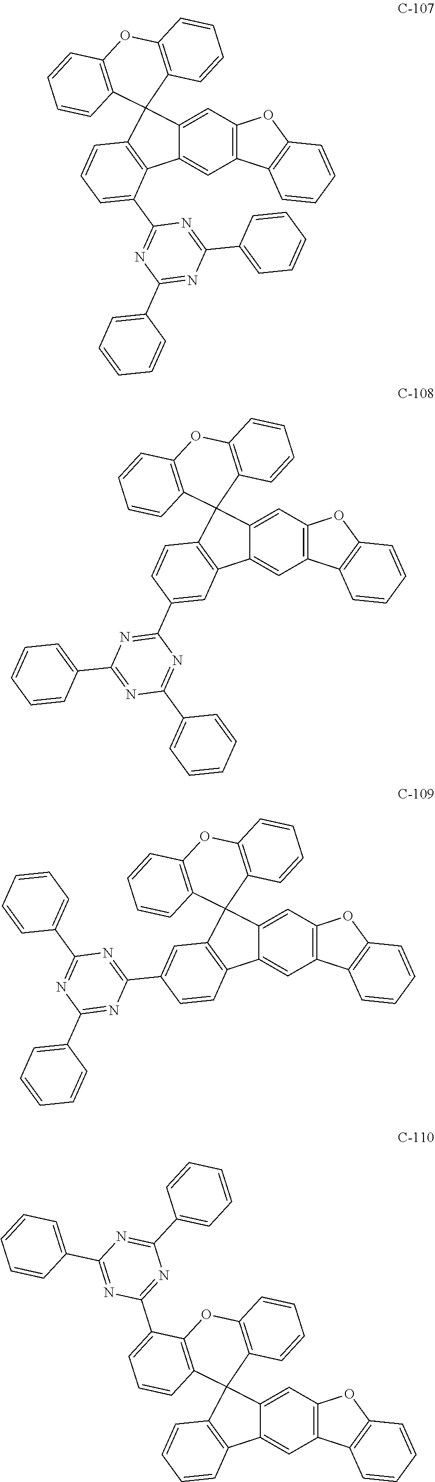 [SMILES: c1ccc(-c2nc(-c3ccccc3)nc(-c3cccc4c3-c3cc5c(cc3C43c4ccccc4Oc4ccccc43)oc3ccccc35)n2)cc1 ; c1ccc(-c2nc(-c3ccccc3)nc(-c3ccc4c(c3)-c3cc5c(cc3C43c4ccccc4Oc4ccccc43)oc3ccccc35)n2)cc1 ; c1ccc(-c2nc(-c3ccccc3)nc(-c3ccc4c(c3)C3(c5ccccc5Oc5ccccc53)c3cc5oc6ccccc6c5cc3-4)n2)cc1 ; c1ccc(-c2nc(-c3ccccc3)nc(-c3cccc4c3Oc3ccccc3C43c4ccccc4-c4cc5c(cc43)oc3ccccc35)n2)cc1]

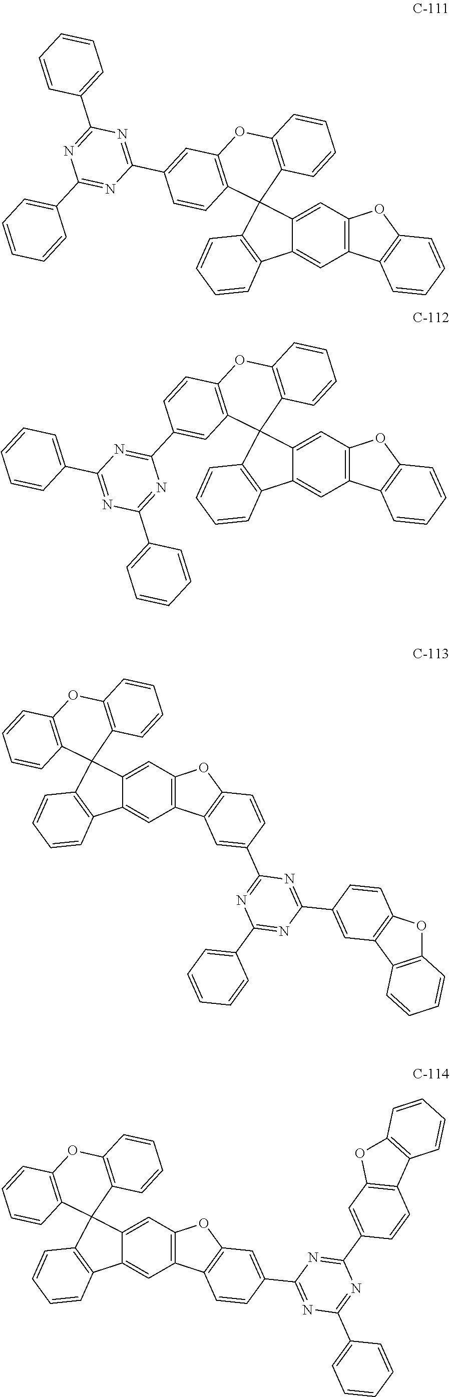 [SMILES: c1ccc(-c2nc(-c3ccccc3)nc(-c3ccc4c(c3)Oc3ccccc3C43c4ccccc4-c4cc5c(cc43)oc3ccccc35)n2)cc1 ; c1ccc(-c2nc(-c3ccccc3)nc(-c3ccc4c(c3)C3(c5ccccc5O4)c4ccccc4-c4cc5c(cc43)oc3ccccc35)n2)cc1 ; c1ccc(-c2nc(-c3ccc4oc5ccccc5c4c3)nc(-c3ccc4oc5cc6c(cc5c4c3)-c3ccccc3C63c4ccccc4Oc4ccccc43)n2)cc1 ; c1ccc(-c2nc(-c3ccc4c(c3)oc3ccccc34)nc(-c3ccc4c(c3)oc3cc5c(cc34)-c3ccccc3C53c4ccccc4Oc4ccccc43)n2)cc1]

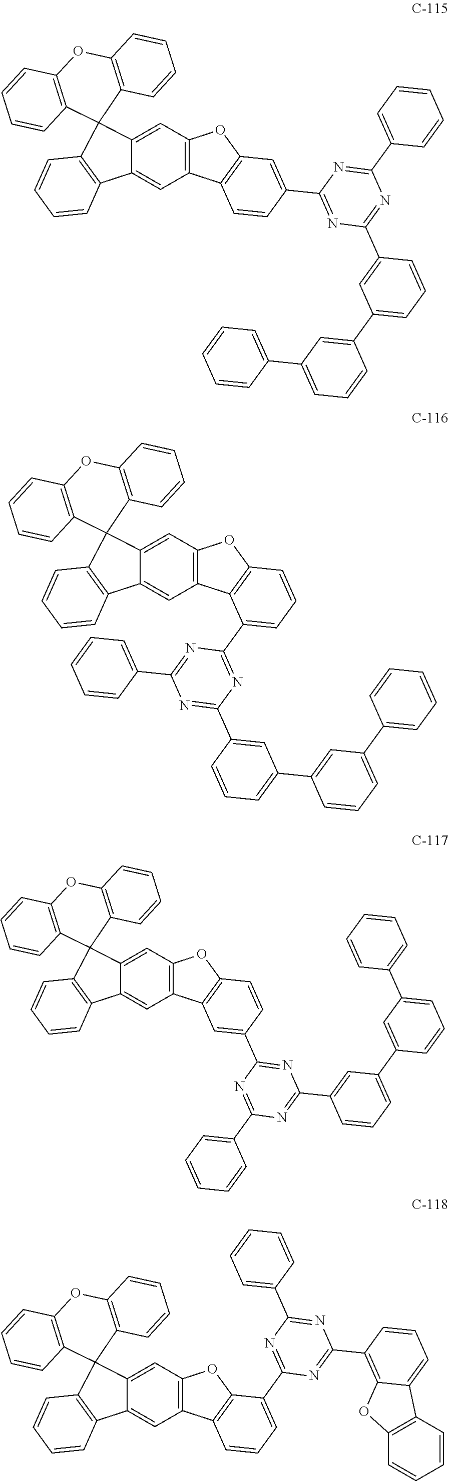

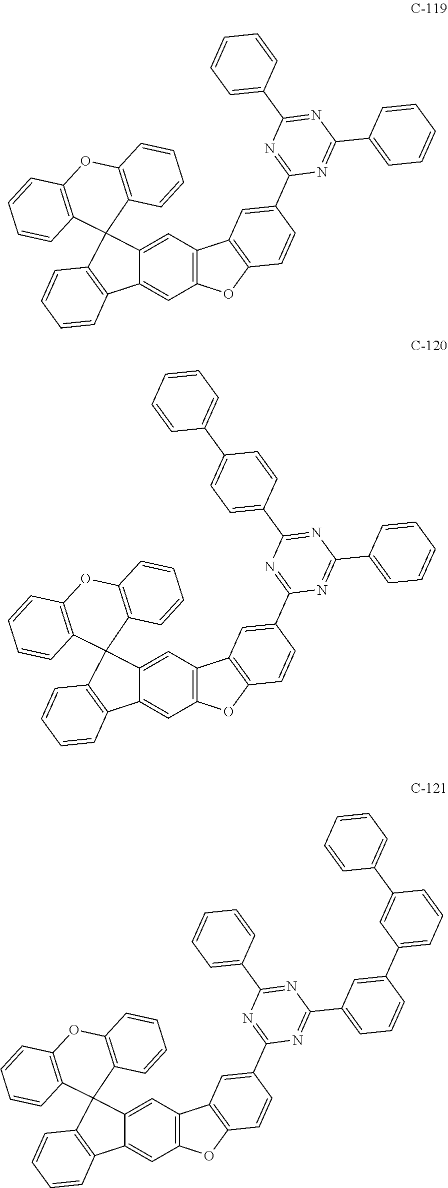 [SMILES: c1ccc(-c2nc(-c3ccccc3)nc(-c3ccc4oc5cc6c(cc5c4c3)C3(c4ccccc4Oc4ccccc43)c3ccccc3-6)n2)cc1 ; c1ccc(-c2ccc(-c3nc(-c4ccccc4)nc(-c4ccc5oc6cc7c(cc6c5c4)C4(c5ccccc5Oc5ccccc54)c4ccccc4-7)n3)cc2)cc1 ; c1ccc(-c2cccc(-c3cccc(-c4nc(-c5ccccc5)nc(-c5ccc6oc7cc8c(cc7c6c5)C5(c6ccccc6Oc6ccccc65)c5ccccc5-8)n4)c3)c2)cc1]

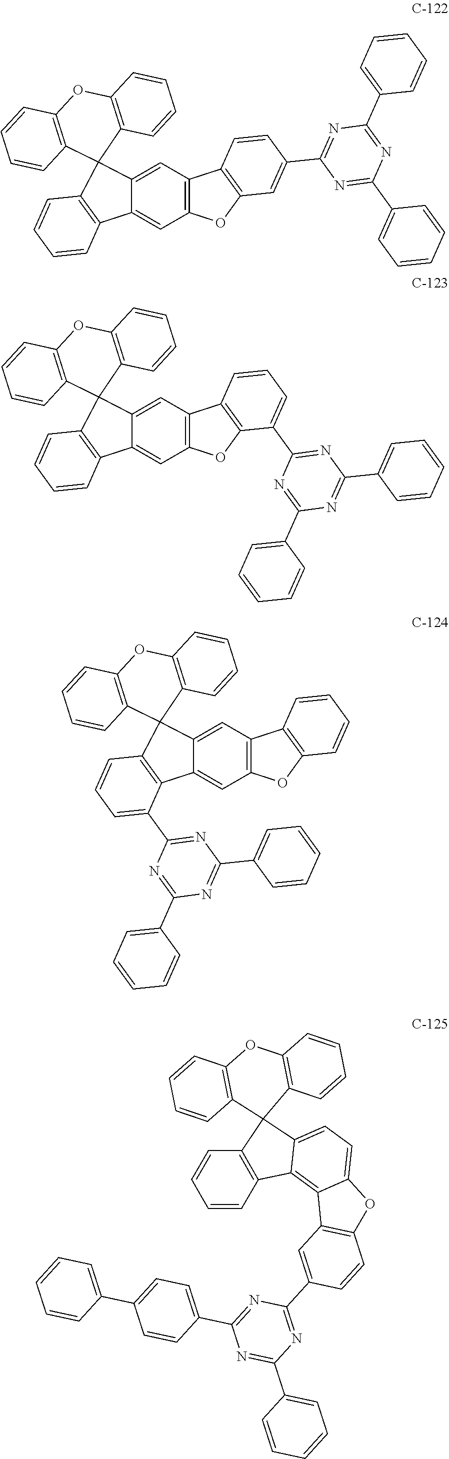 [SMILES: c1ccc(-c2nc(-c3ccccc3)nc(-c3ccc4c(c3)oc3cc5c(cc34)C3(c4ccccc4Oc4ccccc43)c3ccccc3-5)n2)cc1 ; c1ccc(-c2nc(-c3ccccc3)nc(-c3cccc4c3oc3cc5c(cc34)C3(c4ccccc4Oc4ccccc43)c3ccccc3-5)n2)cc1 ; c1ccc(-c2nc(-c3ccccc3)nc(-c3cccc4c3-c3cc5oc6ccccc6c5cc3C43c4ccccc4Oc4ccccc43)n2)cc1 ; c1ccc(-c2ccc(-c3nc(-c4ccccc4)nc(-c4ccc5oc6ccc7c(c6c5c4)-c4ccccc4C74c5ccccc5Oc5ccccc54)n3)cc2)cc1]

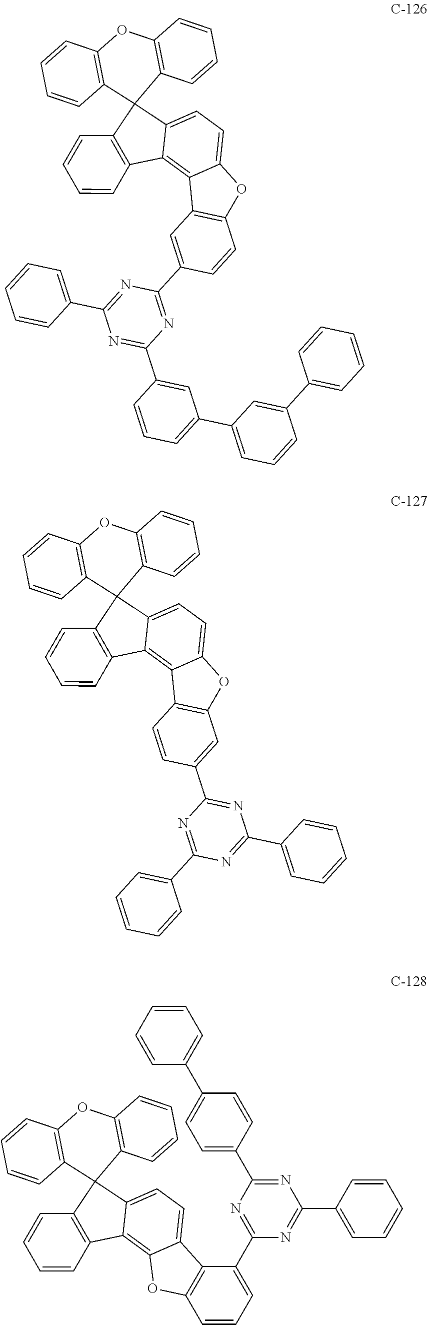 [SMILES: c1ccc(-c2cccc(-c3cccc(-c4nc(-c5ccccc5)nc(-c5ccc6oc7ccc8c(c7c6c5)-c5ccccc5C85c6ccccc6Oc6ccccc65)n4)c3)c2)cc1 ; c1ccc(-c2nc(-c3ccccc3)nc(-c3ccc4c(c3)oc3ccc5c(c34)-c3ccccc3C53c4ccccc4Oc4ccccc43)n2)cc1 ; c1ccc(-c2ccc(-c3nc(-c4ccccc4)nc(-c4cccc5oc6c7c(ccc6c45)C4(c5ccccc5Oc5ccccc54)c4ccccc4-7)n3)cc2)cc1]

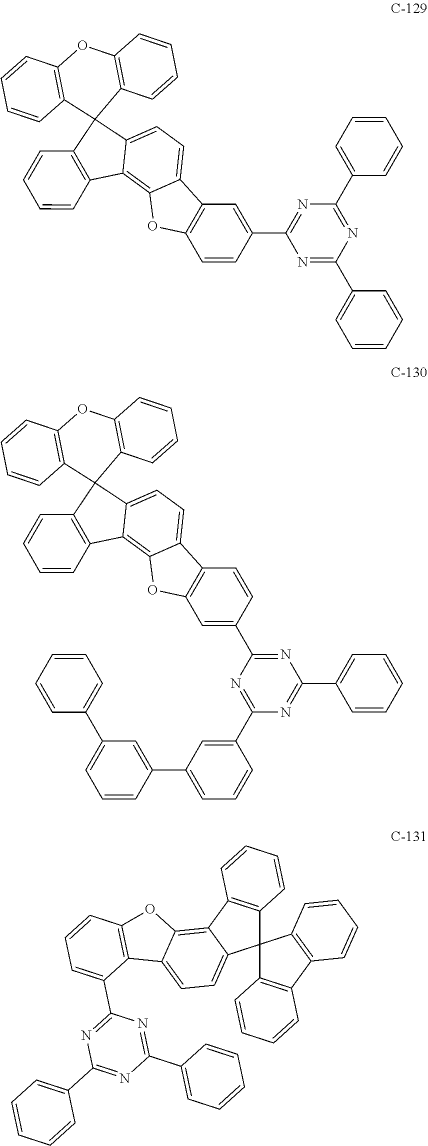 [SMILES: c1ccc(-c2nc(-c3ccccc3)nc(-c3ccc4oc5c6c(ccc5c4c3)C3(c4ccccc4Oc4ccccc43)c3ccccc3-6)n2)cc1 ; c1ccc(-c2cccc(-c3cccc(-c4nc(-c5ccccc5)nc(-c5ccc6c(c5)oc5c7c(ccc56)C5(c6ccccc6Oc6ccccc65)c5ccccc5-7)n4)c3)c2)cc1 ; c1ccc(-c2nc(-c3ccccc3)nc(-c3cccc4oc5c6c(ccc5c34)C3(c4ccccc4-c4ccccc43)c3ccccc3-6)n2)cc1]

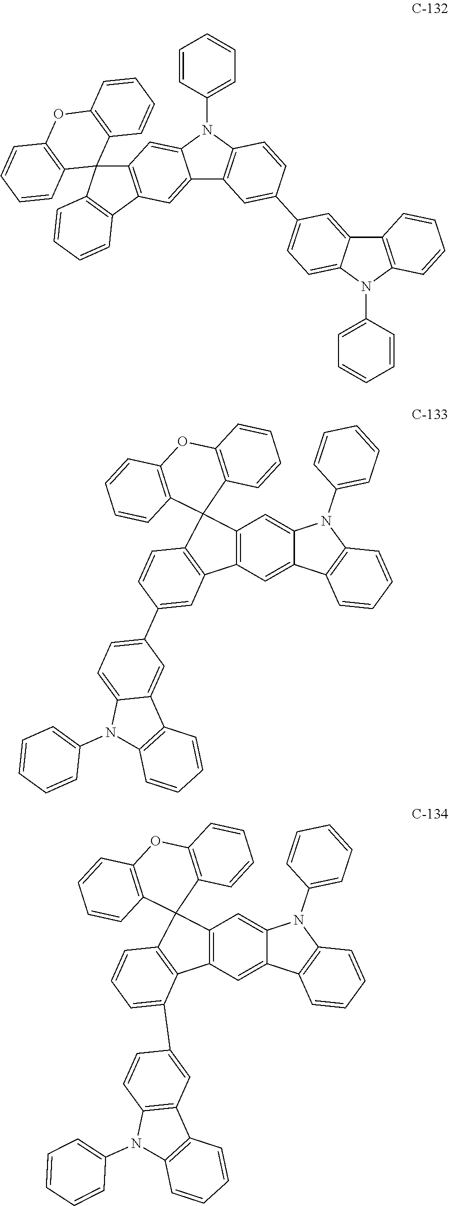

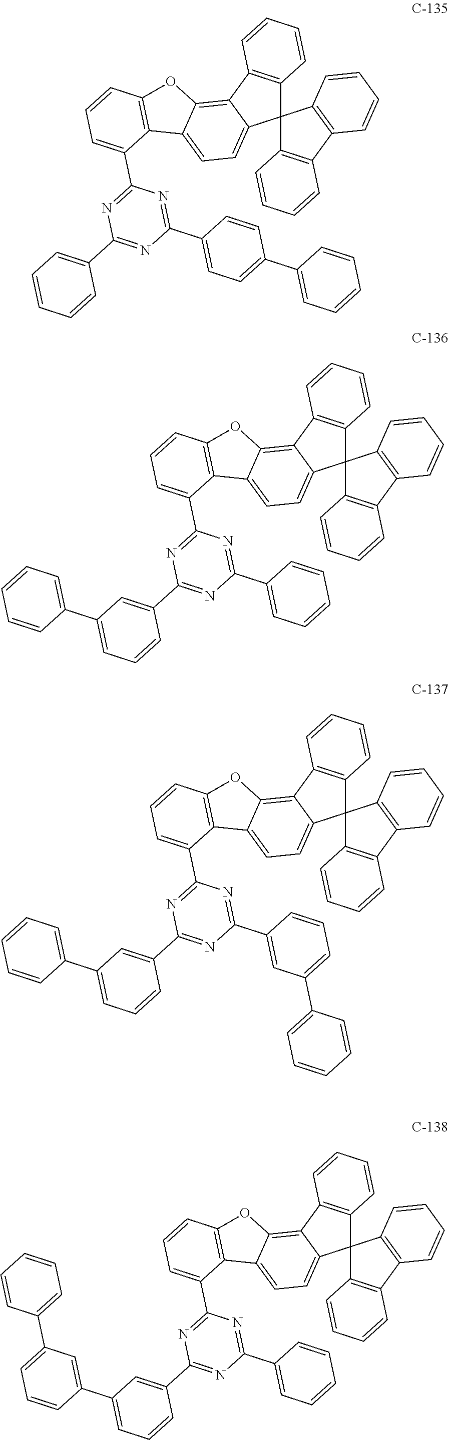 [SMILES: c1ccc(-c2ccc(-c3nc(-c4ccccc4)nc(-c4cccc5oc6c7c(ccc6c45)C4(c5ccccc5-c5ccccc54)c4ccccc4-7)n3)cc2)cc1 ; c1ccc(-c2cccc(-c3nc(-c4ccccc4)nc(-c4cccc5oc6c7c(ccc6c45)C4(c5ccccc5-c5ccccc54)c4ccccc4-7)n3)c2)cc1 ; c1ccc(-c2cccc(-c3nc(-c4cccc(-c5ccccc5)c4)nc(-c4cccc5oc6c7c(ccc6c45)C4(c5ccccc5-c5ccccc54)c4ccccc4-7)n3)c2)cc1 ; c1ccc(-c2cccc(-c3cccc(-c4nc(-c5ccccc5)nc(-c5cccc6oc7c8c(ccc7c56)C5(c6ccccc6-c6ccccc65)c5ccccc5-8)n4)c3)c2)cc1]

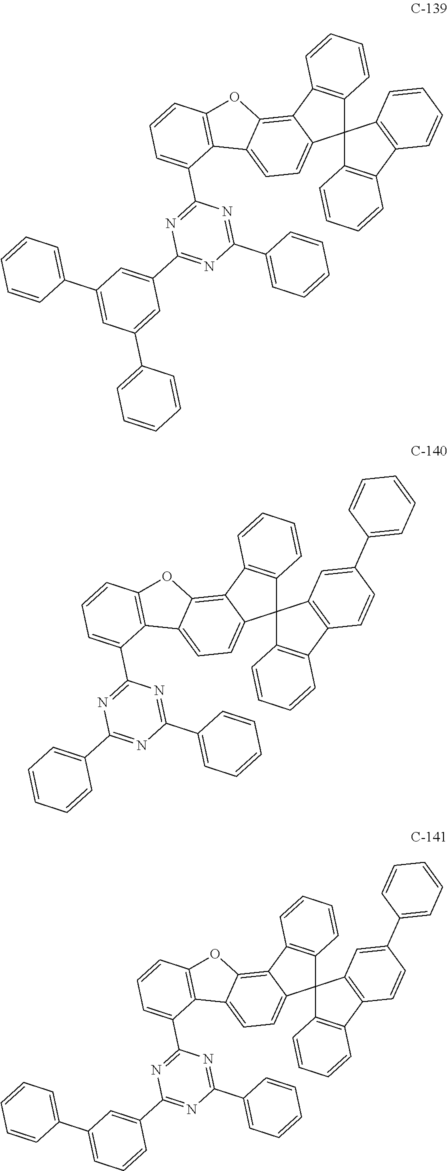 [SMILES: c1ccc(-c2cc(-c3ccccc3)cc(-c3nc(-c4ccccc4)nc(-c4cccc5oc6c7c(ccc6c45)C4(c5ccccc5-c5ccccc54)c4ccccc4-7)n3)c2)cc1 ; c1ccc(-c2ccc3c(c2)C2(c4ccccc4-3)c3ccccc3-c3c2ccc2c3oc3cccc(-c4nc(-c5ccccc5)nc(-c5ccccc5)n4)c32)cc1 ; c1ccc(-c2cccc(-c3nc(-c4ccccc4)nc(-c4cccc5oc6c7c(ccc6c45)C4(c5ccccc5-c5ccc(-c6ccccc6)cc54)c4ccccc4-7)n3)c2)cc1]

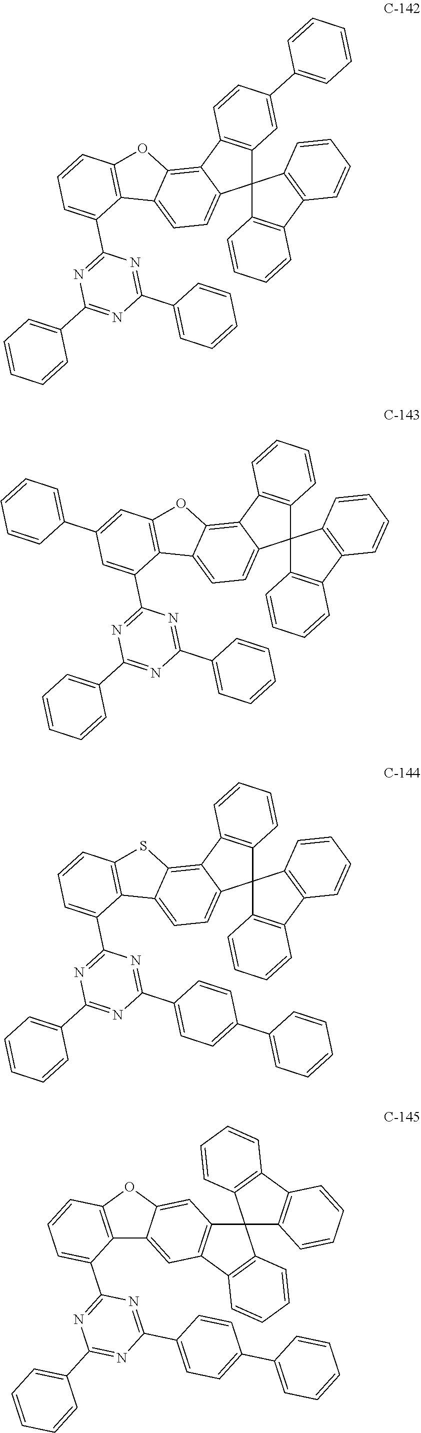 [SMILES: c1ccc(-c2ccc3c(c2)C2(c4ccccc4-c4ccccc42)c2ccc4c(oc5cccc(-c6nc(-c7ccccc7)nc(-c7ccccc7)n6)c54)c2-3)cc1 ; c1ccc(-c2cc(-c3nc(-c4ccccc4)nc(-c4ccccc4)n3)c3c(c2)oc2c4c(ccc23)C2(c3ccccc3-c3ccccc32)c2ccccc2-4)cc1 ; c1ccc(-c2ccc(-c3nc(-c4ccccc4)nc(-c4cccc5sc6c7c(ccc6c45)C4(c5ccccc5-c5ccccc54)c4ccccc4-7)n3)cc2)cc1 ; c1ccc(-c2ccc(-c3nc(-c4ccccc4)nc(-c4cccc5oc6cc7c(cc6c45)-c4ccccc4C74c5ccccc5-c5ccccc54)n3)cc2)cc1]

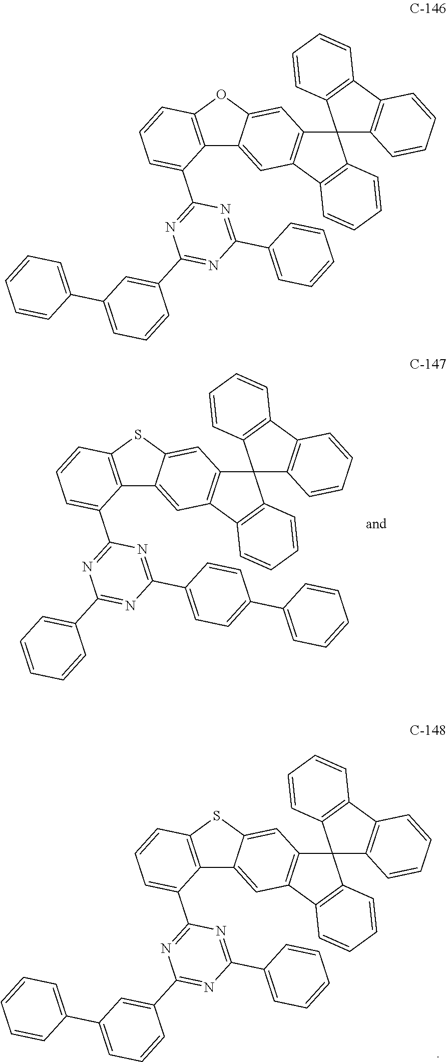 [SMILES: c1ccc(-c2cccc(-c3nc(-c4ccccc4)nc(-c4cccc5oc6cc7c(cc6c45)-c4ccccc4C74c5ccccc5-c5ccccc54)n3)c2)cc1 ; c1ccc(-c2ccc(-c3nc(-c4ccccc4)nc(-c4cccc5sc6cc7c(cc6c45)-c4ccccc4C74c5ccccc5-c5ccccc54)n3)cc2)cc1 ; c1ccc(-c2cccc(-c3nc(-c4ccccc4)nc(-c4cccc5sc6cc7c(cc6c45)-c4ccccc4C74c5ccccc5-c5ccccc54)n3)c2)cc1]

6. The plurality of host materials according to claim 1, wherein the compound represented by formula 2 is at least one selected from the following compounds:

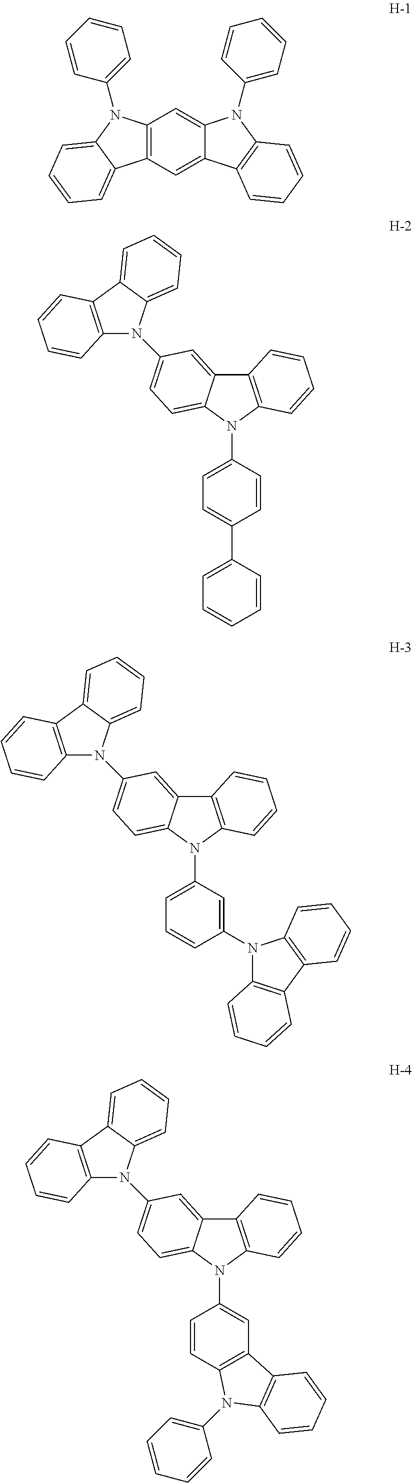 [SMILES: c1ccc(-n2c3ccccc3c3cc4c5ccccc5n(-c5ccccc5)c4cc32)cc1 ; c1ccc(-c2ccc(-n3c4ccccc4c4cc(-n5c6ccccc6c6ccccc65)ccc43)cc2)cc1 ; c1cc(-n2c3ccccc3c3ccccc32)cc(-n2c3ccccc3c3cc(-n4c5ccccc5c5ccccc54)ccc32)c1 ; c1ccc(-n2c3ccccc3c3cc(-n4c5ccccc5c5cc(-n6c7ccccc7c7ccccc76)ccc54)ccc32)cc1]

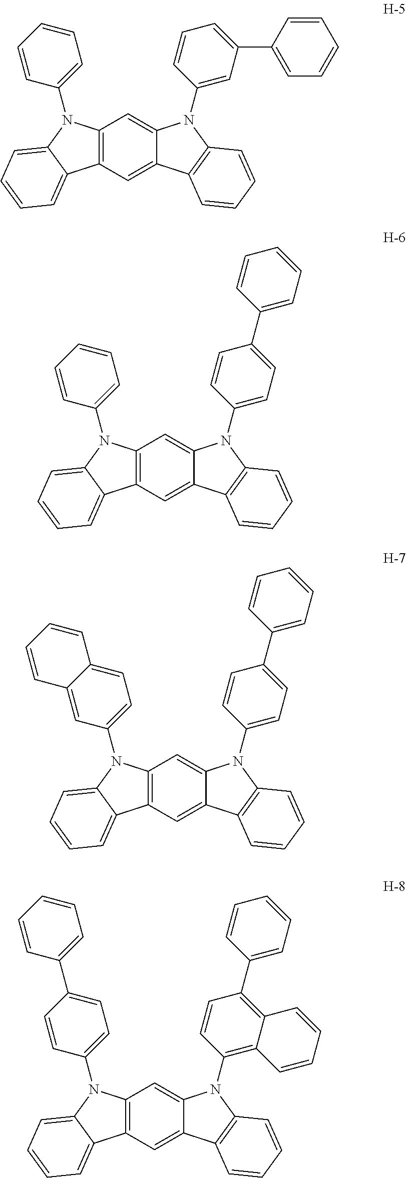

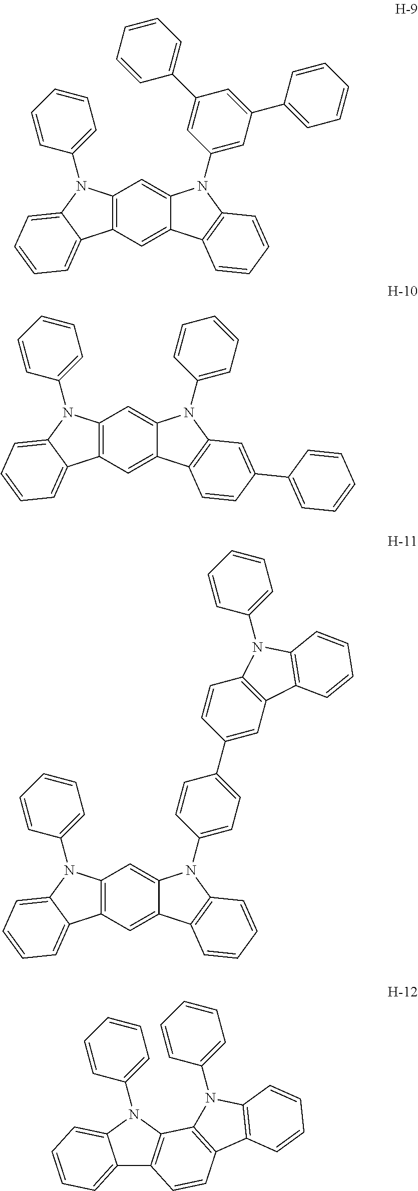

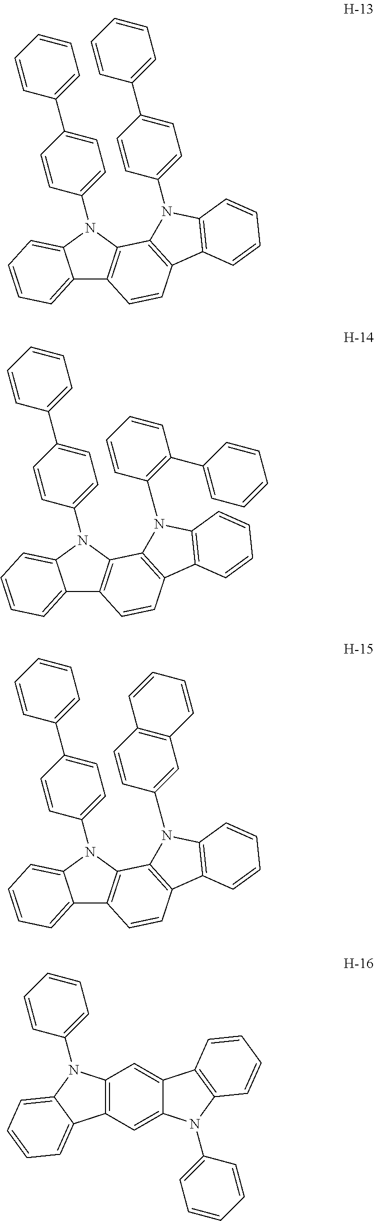

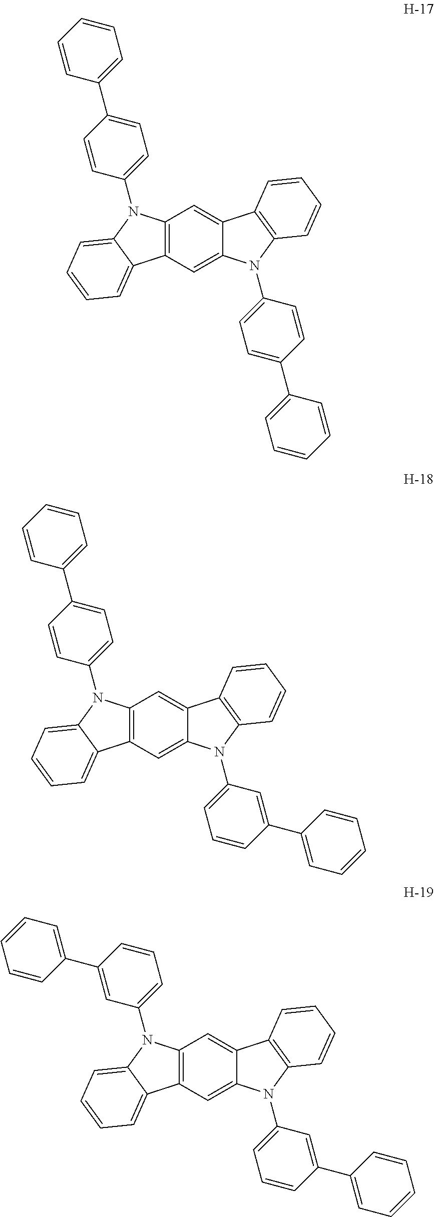 [SMILES: c1ccc(-c2ccc(-n3c4ccccc4c4cc5c(cc43)c3ccccc3n5-c3ccc(-c4ccccc4)cc3)cc2)cc1 ; c1ccc(-c2ccc(-n3c4ccccc4c4cc5c(cc43)c3ccccc3n5-c3cccc(-c4ccccc4)c3)cc2)cc1 ; c1ccc(-c2cccc(-n3c4ccccc4c4cc5c(cc43)c3ccccc3n5-c3cccc(-c4ccccc4)c3)c2)cc1]

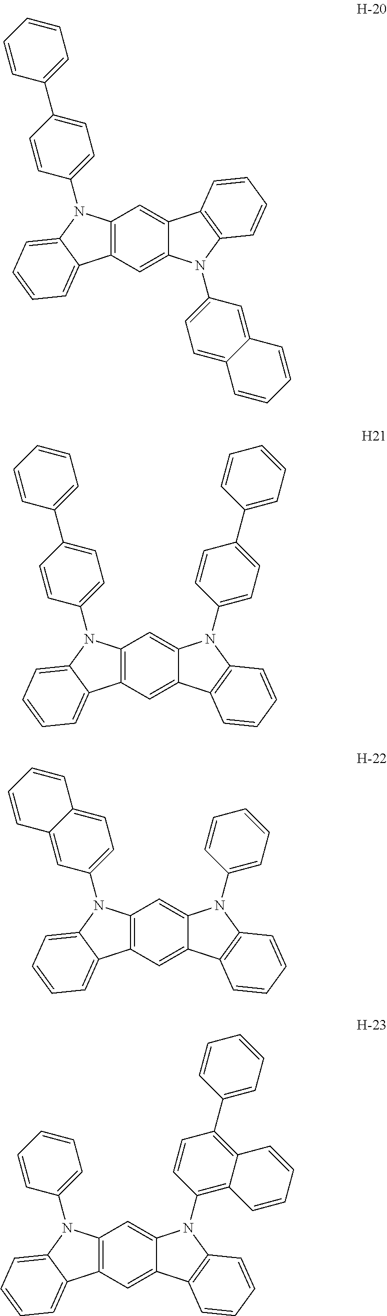

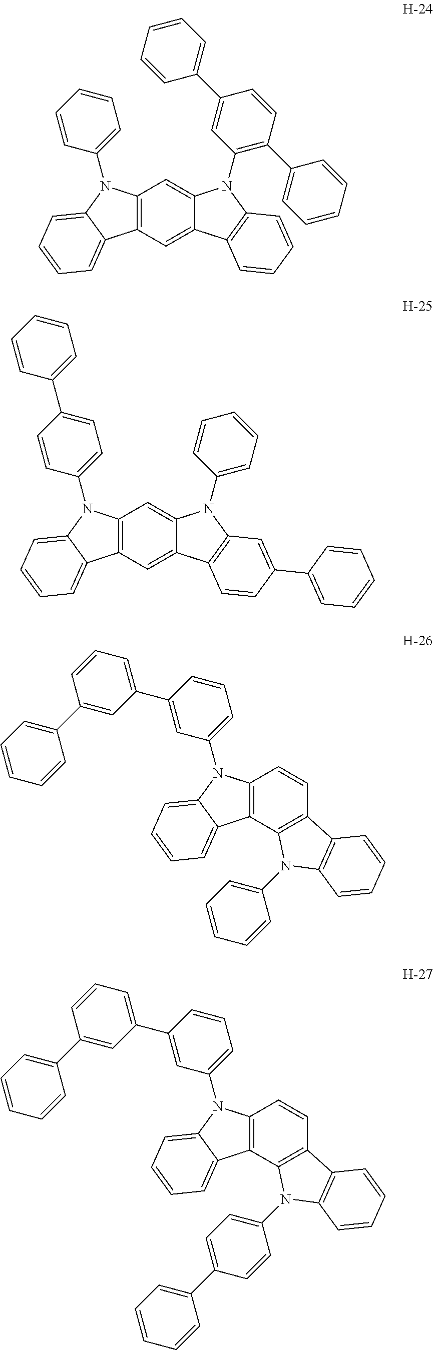 [SMILES: c1ccc(-c2ccc(-c3ccccc3)c(-n3c4ccccc4c4cc5c6ccccc6n(-c6ccccc6)c5cc43)c2)cc1 ; c1ccc(-c2ccc(-n3c4ccccc4c4cc5c6ccc(-c7ccccc7)cc6n(-c6ccccc6)c5cc43)cc2)cc1 ; c1ccc(-c2cccc(-c3cccc(-n4c5ccccc5c5c4ccc4c6ccccc6n(-c6ccccc6)c45)c3)c2)cc1 ; c1ccc(-c2ccc(-n3c4ccccc4c4ccc5c(c6ccccc6n5-c5cccc(-c6cccc(-c7ccccc7)c6)c5)c43)cc2)cc1]

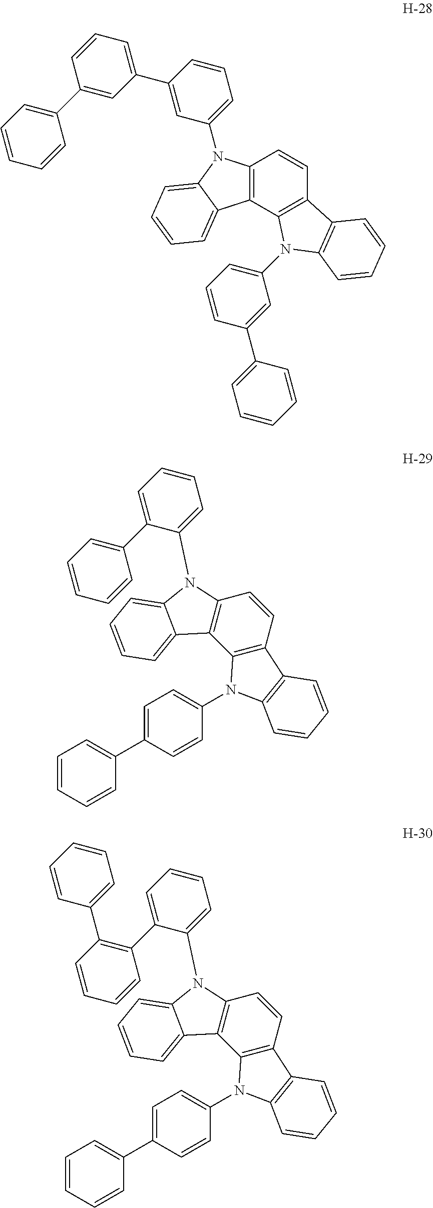

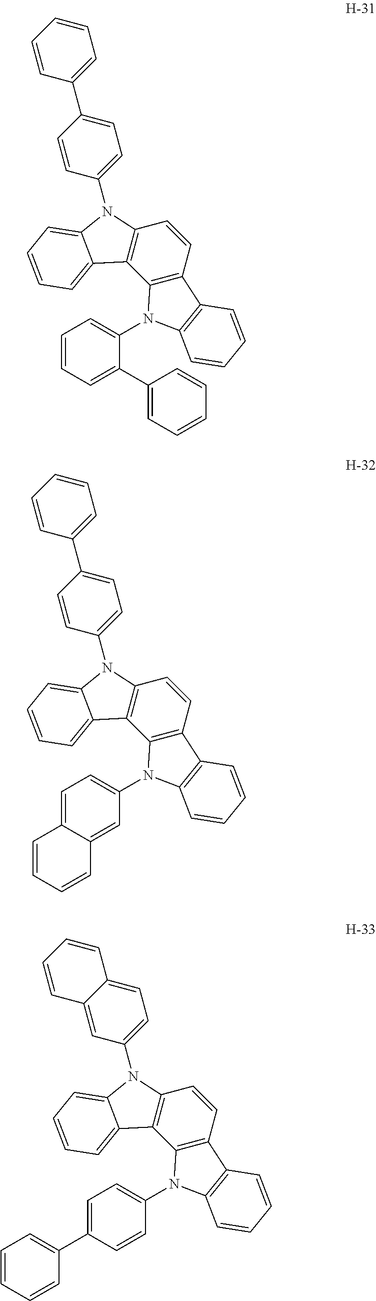 [SMILES: c1ccc(-c2ccc(-n3c4ccccc4c4c3ccc3c5ccccc5n(-c5ccccc5-c5ccccc5)c34)cc2)cc1 ; c1ccc(-c2ccc(-n3c4ccccc4c4c3ccc3c5ccccc5n(-c5ccc6ccccc6c5)c34)cc2)cc1 ; c1ccc(-c2ccc(-n3c4ccccc4c4ccc5c(c6ccccc6n5-c5ccc6ccccc6c5)c43)cc2)cc1]

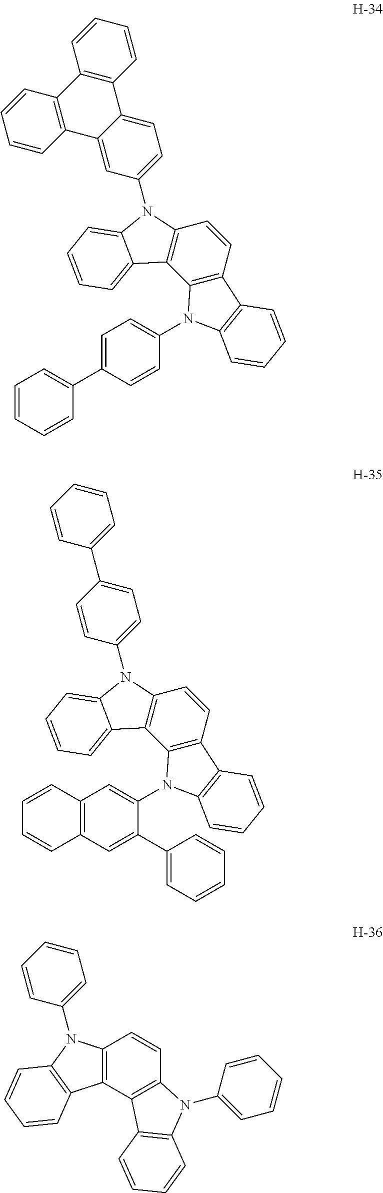 [SMILES: c1ccc(-c2ccc(-n3c4ccccc4c4ccc5c(c6ccccc6n5-c5ccc6c7ccccc7c7ccccc7c6c5)c43)cc2)cc1 ; c1ccc(-c2ccc(-n3c4ccccc4c4c3ccc3c5ccccc5n(-c5cc6ccccc6cc5-c5ccccc5)c34)cc2)cc1 ; c1ccc(-n2c3ccccc3c3c4c5ccccc5n(-c5ccccc5)c4ccc32)cc1]

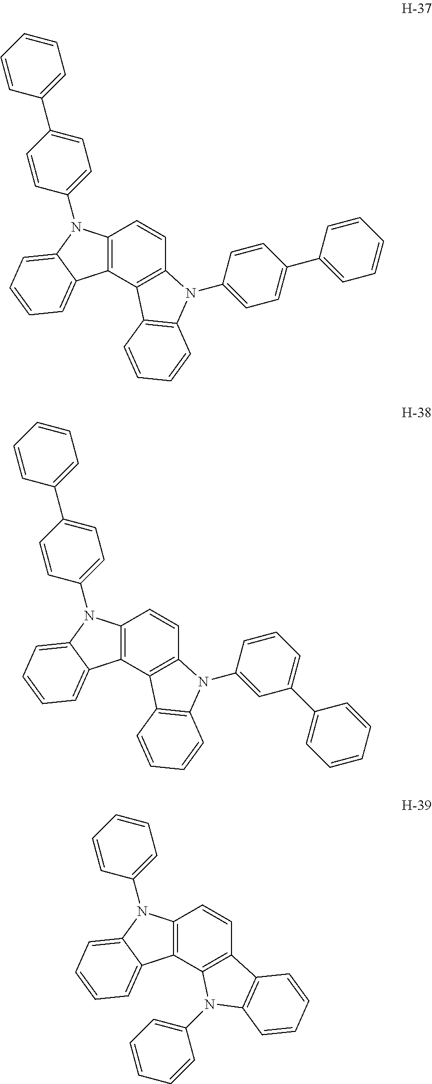 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-n2c3ccccc3c3c2ccc2c4ccccc4n(-c4ccccc4)c23)cc1]

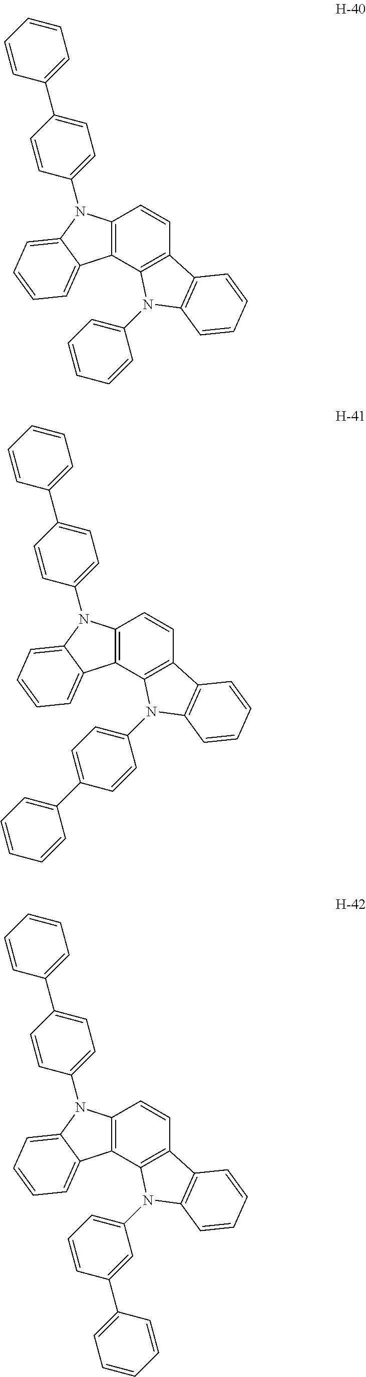 [SMILES: c1ccc(-c2ccc(-n3c4ccccc4c4c3ccc3c5ccccc5n(-c5ccccc5)c34)cc2)cc1 ; c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4c3ccc3c5ccccc5n(-c5cccc(-c6ccccc6)c5)c34)cc2)cc1]

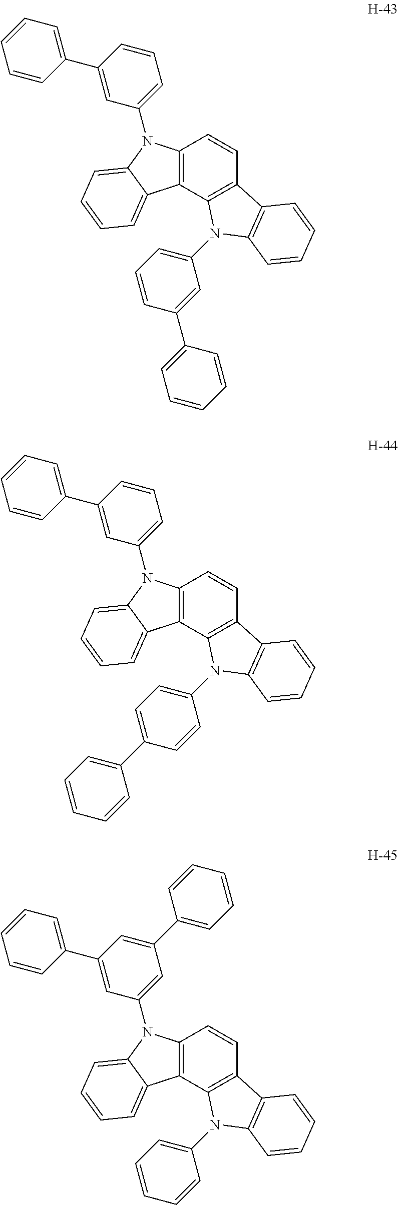 [SMILES: c1ccc(-c2cccc(-n3c4ccccc4c4c3ccc3c5ccccc5n(-c5cccc(-c6ccccc6)c5)c34)c2)cc1 ; c1ccc(-c2ccc(-n3c4ccccc4c4ccc5c(c6ccccc6n5-c5cccc(-c6ccccc6)c5)c43)cc2)cc1 ; c1ccc(-c2cc(-c3ccccc3)cc(-n3c4ccccc4c4c3ccc3c5ccccc5n(-c5ccccc5)c34)c2)cc1]

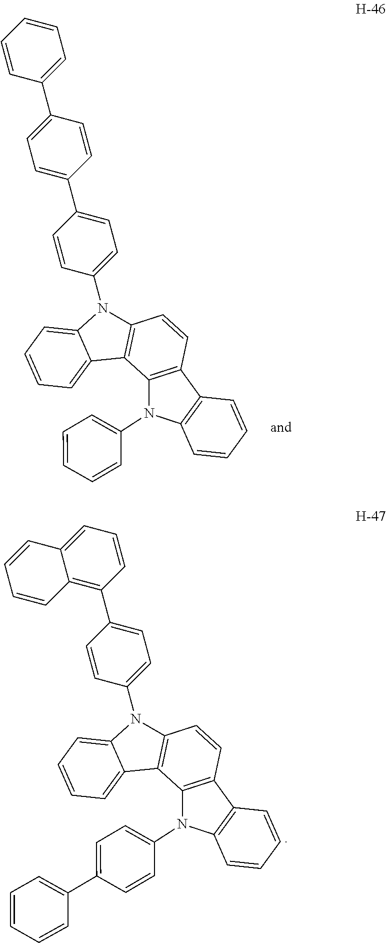

7. An organic electroluminescent device comprising an anode, a cathode, and at least one light-emitting layer between the anode and the cathode, wherein at least one layer of the light-emitting layers comprises the plurality of host materials according to claim 1.

8. An organic electroluminescent compound represented by the following formula 1: 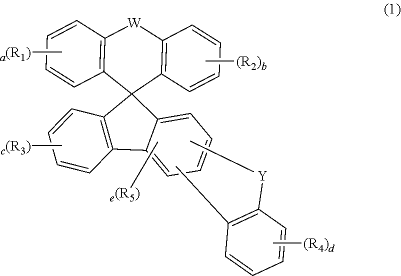
wherein
W represents a single bond, O, S, CR₆R₇, or N-L-R₈;
Y represents O, S, CR₆R₇, or N-L-R₈;
L, each independently, represents a single bond, a substituted or unsubstituted (C6-C30)arylene, or a substituted or unsubstituted (3- to 30-membered)heteroarylene;
R₁ to R₈, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arysilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or adjacent ones of R₁ to R₈ may be linked to each other to form a ring(s);
at least one of R₁ to R₅ comprises a substituted or unsubstituted nitrogen-containing (3- to 30-membered)heteroaryl;
with the proviso that if Y represents N-L-R₈, at least one of R₃ to R₅ comprises a substituted or unsubstituted nitrogen-containing (3- to 30-membered)heteroaryl; if Y N-L-R₈, R₄ is not a 9-phenylcarbazolyl or a (9-carbazolyl)phenyl; and
a to d, each independently, represent an integer of 1 to 4; and e represents an integer of 1 or 2; where each of a to e is an integer of 2 or more, each of R₁, each of R₂, each of R₃, each of R₄, and each of R₅ may be the same or different

9. A plurality of host materials comprising a first host material comprising the compound represented by the formula 1 according to claim 8, and a second host material comprising a compound represented by the following formula 3: 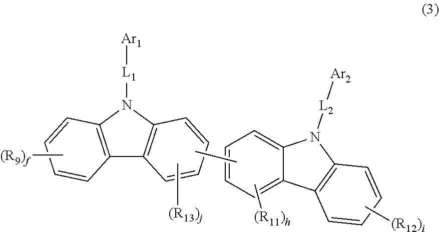
wherein
L₁ and L₂, each independently, represent a single bond, or a substituted or unsubstituted (C6-C30)arylene;
Ar₁ and Ar₂, each independently, represent a substituted or unsubstituted (C6-C30)aryl;
R₉ and R₁₁ to R₁₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arysilyl, a substituted or unsubstituted tri(C6-C30)arylsilyl, a substituted or unsubstituted mono- or di-(C1-C30)alkylamino, a substituted or unsubstituted (C1-C30)alkyl(C6-C30)arylamino, or a substituted or unsubstituted mono- or di-(C6-C30)arylamino; or two or more adjacent R₉'s may be linked to each other to form a ring(s), two or more adjacent R₁₁'s may be linked to each other to form a ring(s), two or more adjacent R₁₂'s may be linked to each other to form a ring(s), and two or more adjacent R₁₃'s may be linked to each other to form a ring(s); and
f and i, each independently, represent an integer of 1 to 4; and h and j, each independently, represent an integer of 1 to 3; where f, h, i, and j is an integer of 2 or more, each of R₉, each of R₁₁, each of R₁₂, and each of R₁₃ may be the same or different.

10. The organic electroluminescent compound according to claim 8, wherein the compound represented by formula 1 is selected from the following compounds:

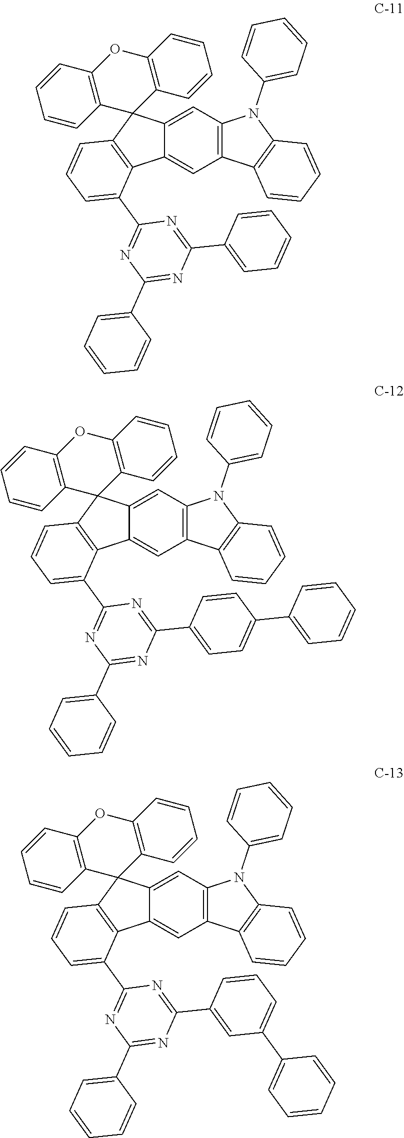

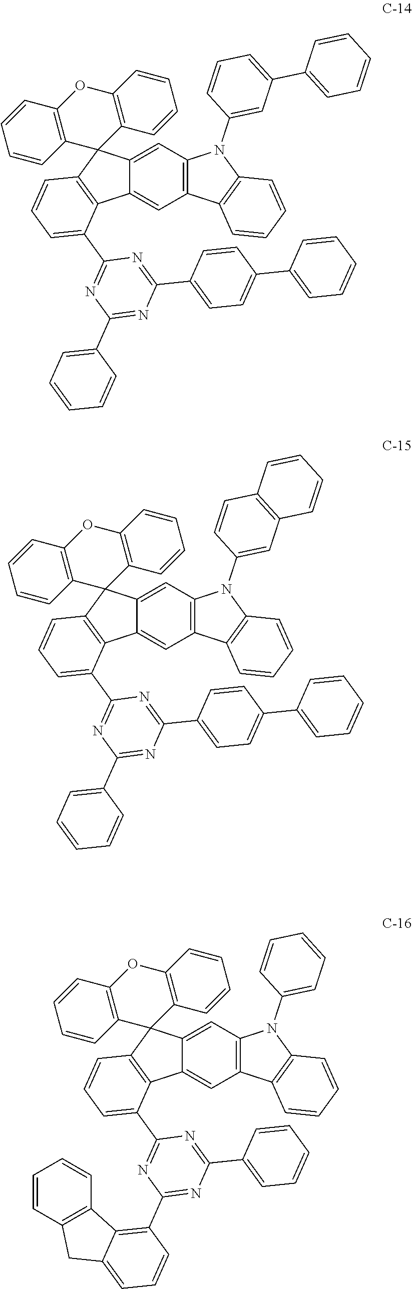 [SMILES: c1ccc(-c2ccc(-c3nc(-c4ccccc4)nc(-c4cccc5c4-c4cc6c7ccccc7n(-c7cccc(-c8ccccc8)c7)c6cc4C54c5ccccc5Oc5ccccc54)n3)cc2)cc1 ; c1ccc(-c2ccc(-c3nc(-c4ccccc4)nc(-c4cccc5c4-c4cc6c7ccccc7n(-c7ccc8ccccc8c7)c6cc4C54c5ccccc5Oc5ccccc54)n3)cc2)cc1 ; c1ccc(-c2nc(-c3cccc4c3-c3ccccc3C4)nc(-c3cccc4c3-c3cc5c6ccccc6n(-c6ccccc6)c5cc3C43c4ccccc4Oc4ccccc43)n2)cc1]

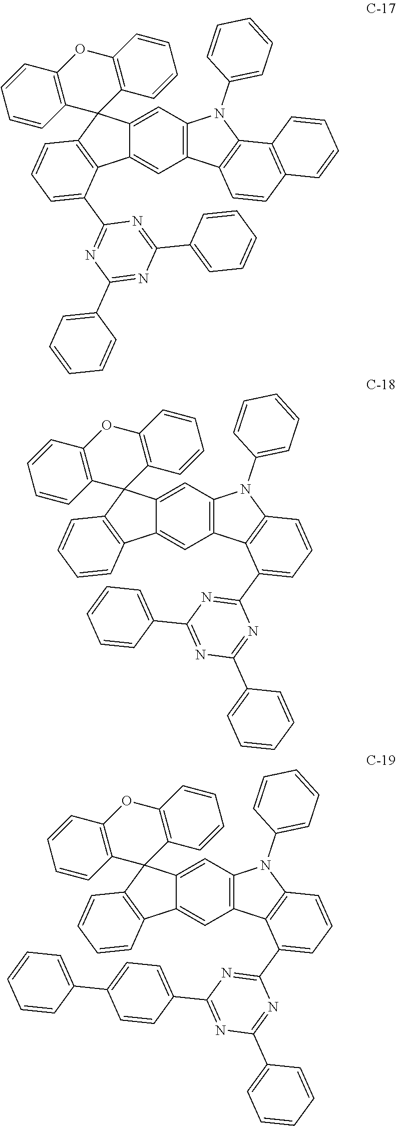

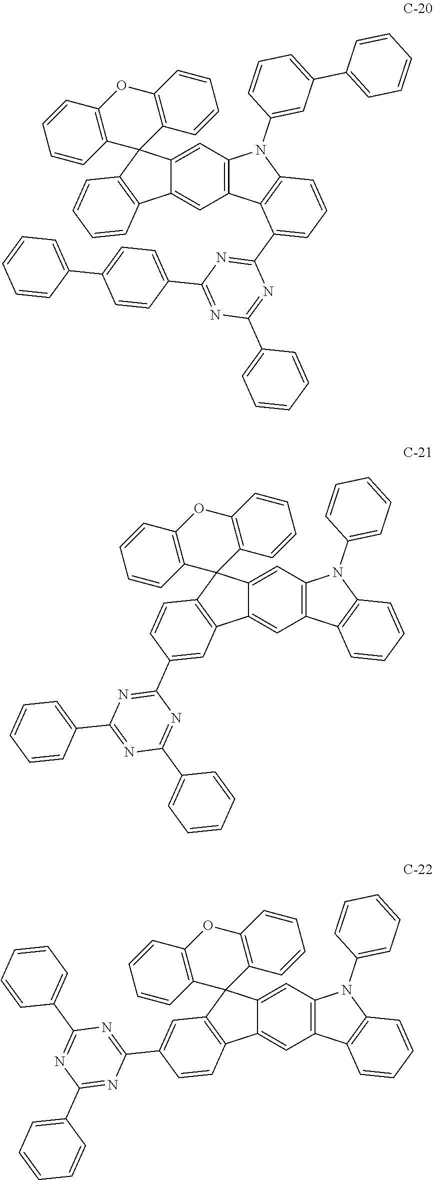 [SMILES: c1ccc(-c2ccc(-c3nc(-c4ccccc4)nc(-c4cccc5c4c4cc6c(cc4n5-c4cccc(-c5ccccc5)c4)C4(c5ccccc5Oc5ccccc54)c4ccccc4-6)n3)cc2)cc1 ; c1ccc(-c2nc(-c3ccccc3)nc(-c3ccc4c(c3)-c3cc5c6ccccc6n(-c6ccccc6)c5cc3C43c4ccccc4Oc4ccccc43)n2)cc1 ; c1ccc(-c2nc(-c3ccccc3)nc(-c3ccc4c(c3)C3(c5ccccc5Oc5ccccc53)c3cc5c(cc3-4)c3ccccc3n5-c3ccccc3)n2)cc1]

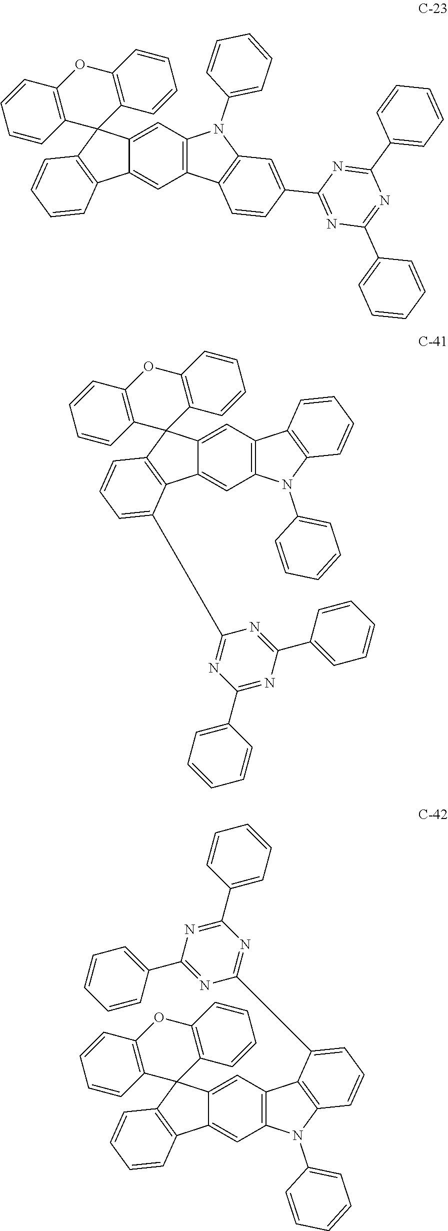

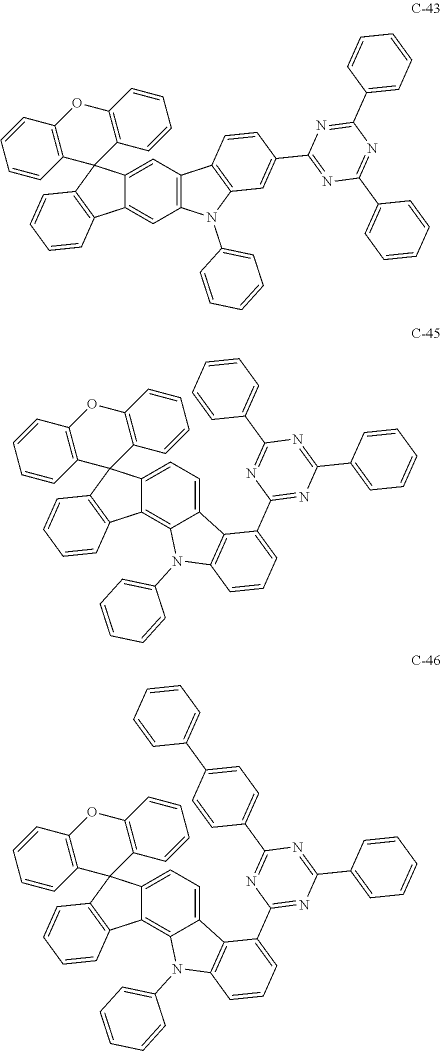

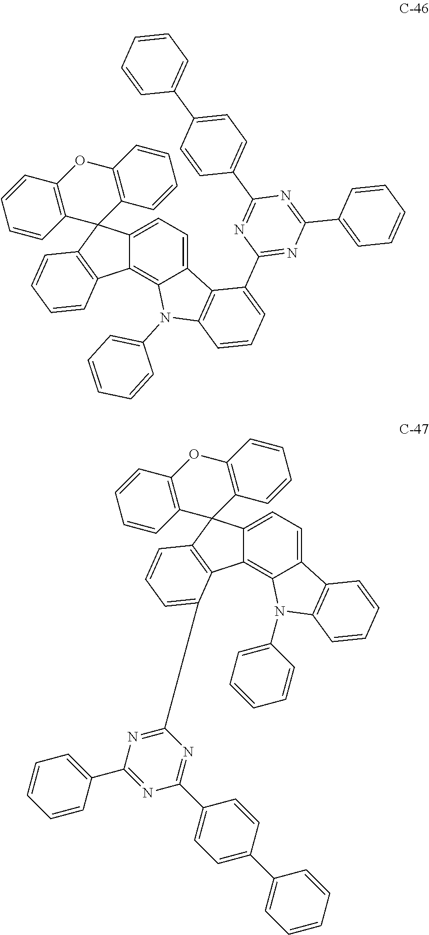 [SMILES: c1ccc(-c2ccc(-c3nc(-c4ccccc4)nc(-c4cccc5c4c4ccc6c(c4n5-c4ccccc4)-c4ccccc4C64c5ccccc5Oc5ccccc54)n3)cc2)cc1 ; c1ccc(-c2ccc(-c3nc(-c4ccccc4)nc(-c4cccc5c4-c4c(ccc6c7ccccc7n(-c7ccccc7)c46)C54c5ccccc5Oc5ccccc54)n3)cc2)cc1]

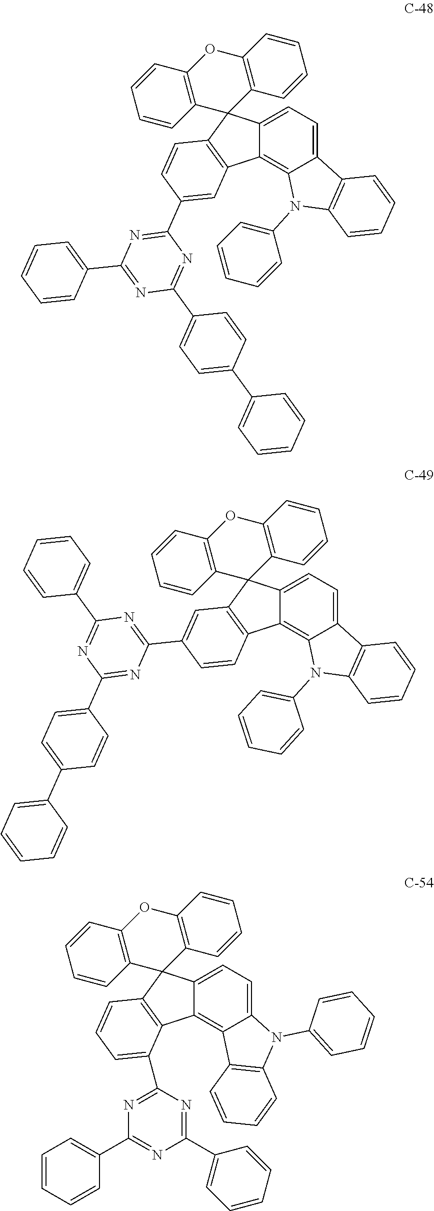 [SMILES: c1ccc(-c2ccc(-c3nc(-c4ccccc4)nc(-c4ccc5c(c4)-c4c(ccc6c7ccccc7n(-c7ccccc7)c46)C54c5ccccc5Oc5ccccc54)n3)cc2)cc1 ; c1ccc(-c2ccc(-c3nc(-c4ccccc4)nc(-c4ccc5c(c4)C4(c6ccccc6Oc6ccccc64)c4ccc6c7ccccc7n(-c7ccccc7)c6c4-5)n3)cc2)cc1 ; c1ccc(-c2nc(-c3ccccc3)nc(-c3cccc4c3-c3c(ccc5c3c3ccccc3n5-c3ccccc3)C43c4ccccc4Oc4ccccc43)n2)cc1]

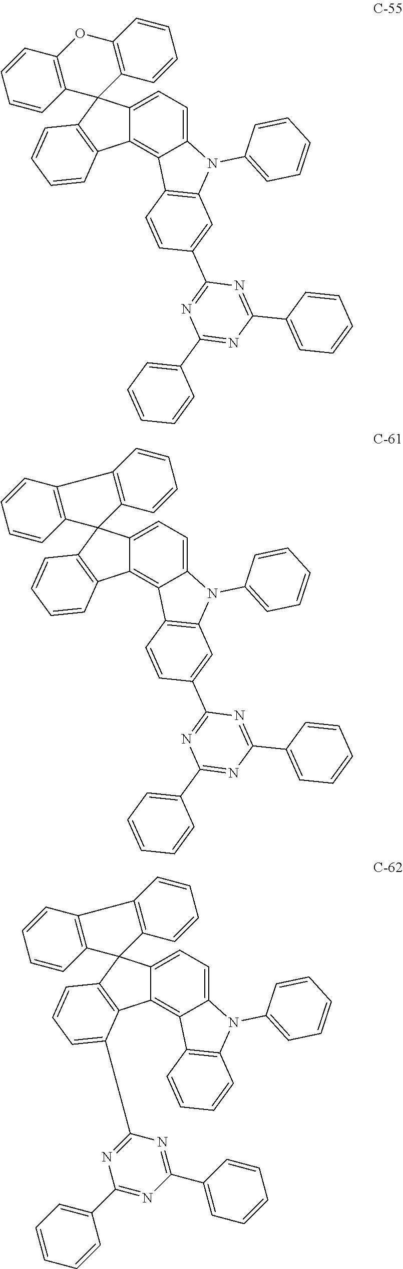

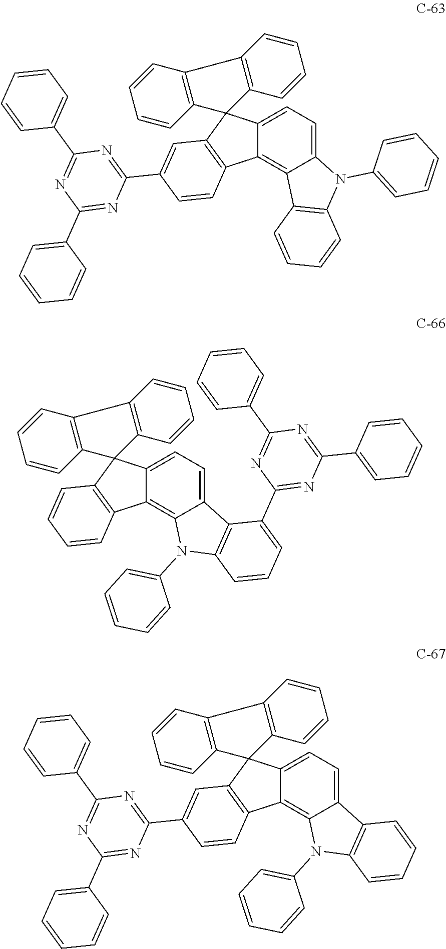 [SMILES: c1ccc(-c2nc(-c3ccccc3)nc(-c3ccc4c(c3)C3(c5ccccc5-c5ccccc53)c3ccc5c(c3-4)c3ccccc3n5-c3ccccc3)n2)cc1 ; c1ccc(-c2nc(-c3ccccc3)nc(-c3cccc4c3c3ccc5c(c3n4-c3ccccc3)-c3ccccc3C53c4ccccc4-c4ccccc43)n2)cc1 ; c1ccc(-c2nc(-c3ccccc3)nc(-c3ccc4c(c3)C3(c5ccccc5-c5ccccc53)c3ccc5c6ccccc6n(-c6ccccc6)c5c3-4)n2)cc1]

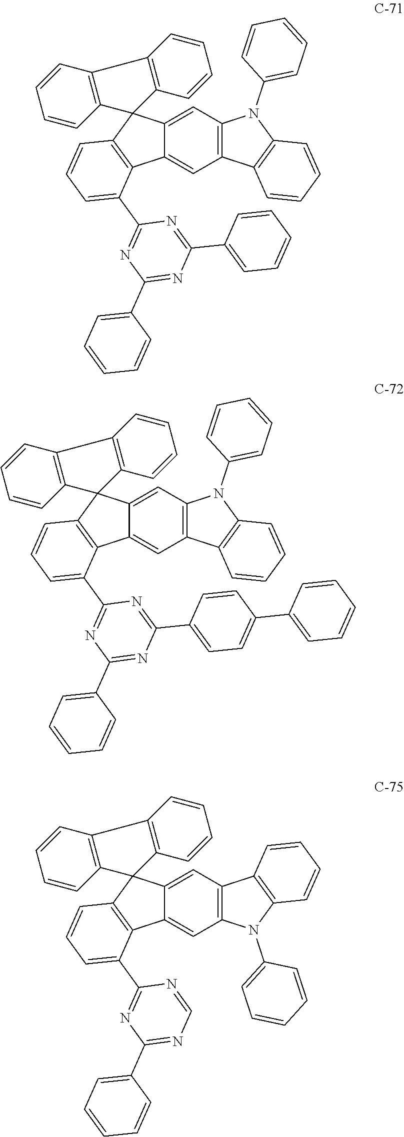

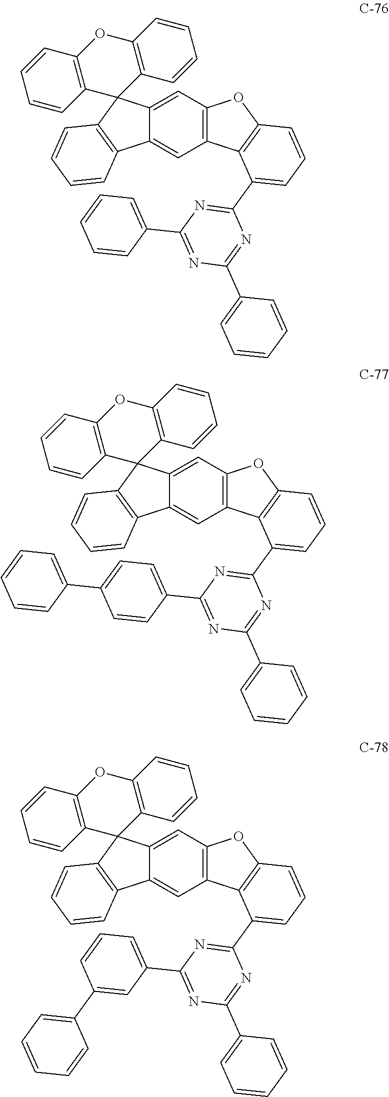

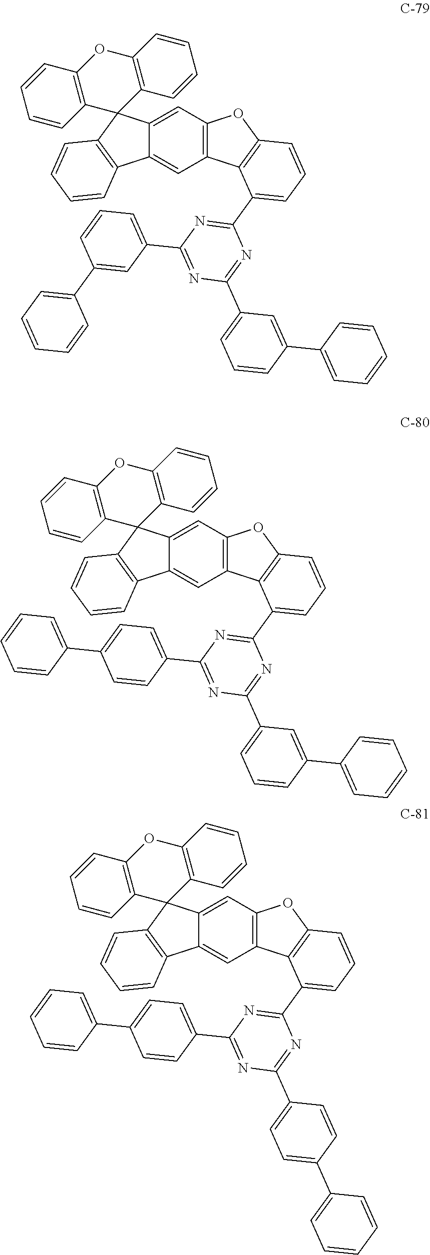 [SMILES: c1ccc(-c2cccc(-c3nc(-c4cccc(-c5ccccc5)c4)nc(-c4cccc5oc6cc7c(cc6c45)-c4ccccc4C74c5ccccc5Oc5ccccc54)n3)c2)cc1 ; c1ccc(-c2ccc(-c3nc(-c4cccc(-c5ccccc5)c4)nc(-c4cccc5oc6cc7c(cc6c45)-c4ccccc4C74c5ccccc5Oc5ccccc54)n3)cc2)cc1 ; c1ccc(-c2ccc(-c3nc(-c4ccc(-c5ccccc5)cc4)nc(-c4cccc5oc6cc7c(cc6c45)-c4ccccc4C74c5ccccc5Oc5ccccc54)n3)cc2)cc1]

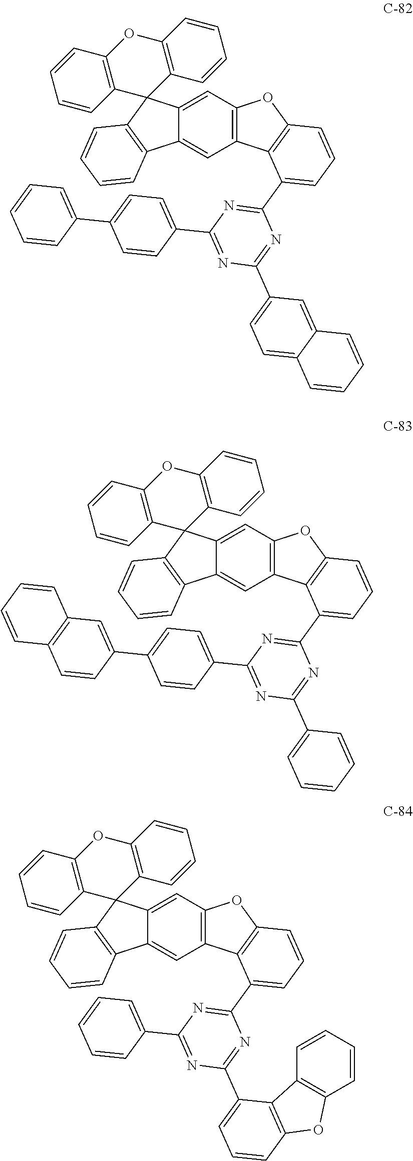 [SMILES: c1ccc(-c2ccc(-c3nc(-c4ccc5ccccc5c4)nc(-c4cccc5oc6cc7c(cc6c45)-c4ccccc4C74c5ccccc5Oc5ccccc54)n3)cc2)cc1 ; c1ccc(-c2nc(-c3ccc(-c4ccc5ccccc5c4)cc3)nc(-c3cccc4oc5cc6c(cc5c34)-c3ccccc3C63c4ccccc4Oc4ccccc43)n2)cc1 ; c1ccc(-c2nc(-c3cccc4oc5ccccc5c34)nc(-c3cccc4oc5cc6c(cc5c34)-c3ccccc3C63c4ccccc4Oc4ccccc43)n2)cc1]

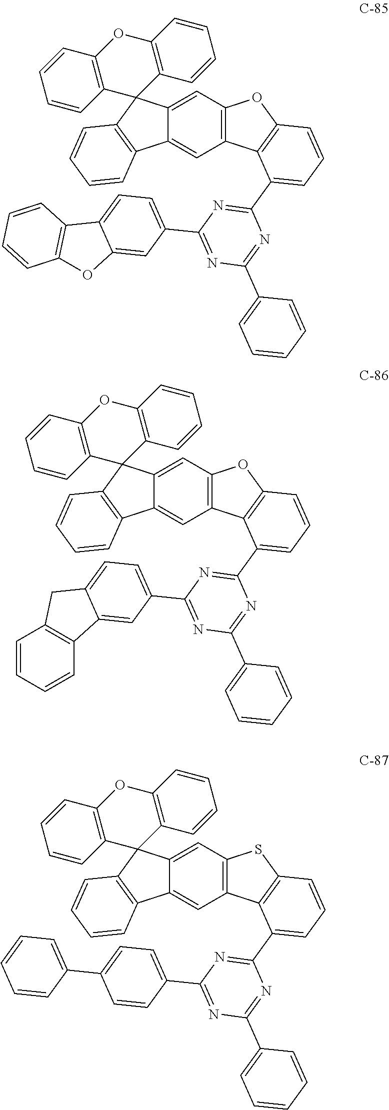 [SMILES: c1ccc(-c2nc(-c3ccc4c(c3)oc3ccccc34)nc(-c3cccc4oc5cc6c(cc5c34)-c3ccccc3C63c4ccccc4Oc4ccccc43)n2)cc1 ; c1ccc(-c2nc(-c3ccc4c(c3)-c3ccccc3C4)nc(-c3cccc4oc5cc6c(cc5c34)-c3ccccc3C63c4ccccc4Oc4ccccc43)n2)cc1 ; c1ccc(-c2ccc(-c3nc(-c4ccccc4)nc(-c4cccc5sc6cc7c(cc6c45)-c4ccccc4C74c5ccccc5Oc5ccccc54)n3)cc2)cc1]

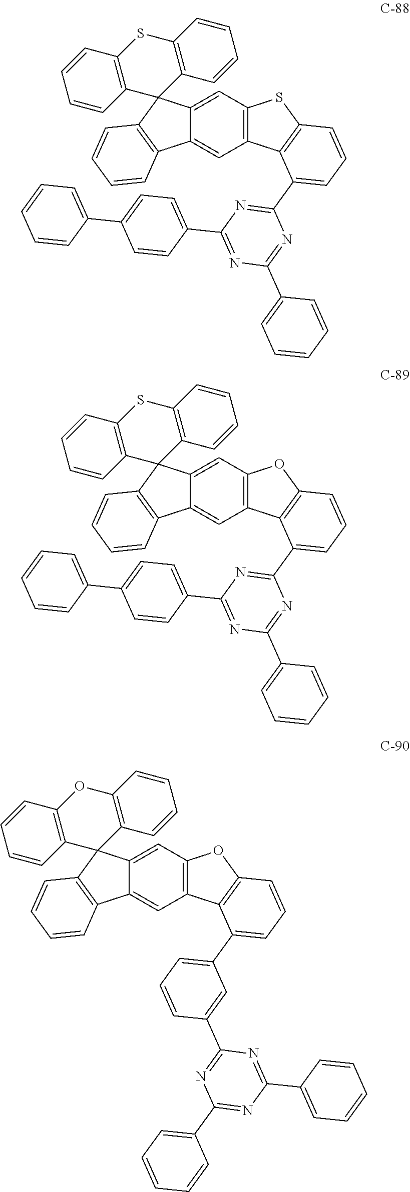 [SMILES: c1ccc(-c2ccc(-c3nc(-c4ccccc4)nc(-c4cccc5sc6cc7c(cc6c45)-c4ccccc4C74c5ccccc5Sc5ccccc54)n3)cc2)cc1 ; c1ccc(-c2ccc(-c3nc(-c4ccccc4)nc(-c4cccc5oc6cc7c(cc6c45)-c4ccccc4C74c5ccccc5Sc5ccccc54)n3)cc2)cc1 ; c1ccc(-c2nc(-c3ccccc3)nc(-c3cccc(-c4cccc5oc6cc7c(cc6c45)-c4ccccc4C74c5ccccc5Oc5ccccc54)c3)n2)cc1]

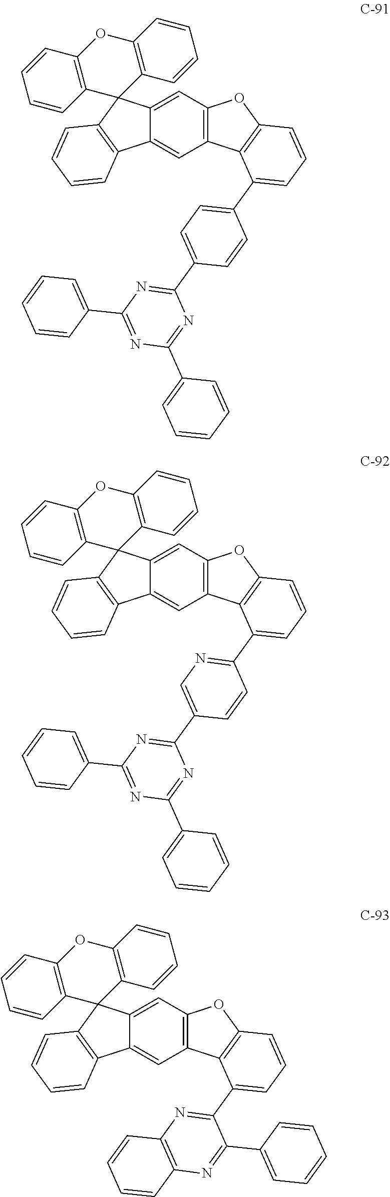

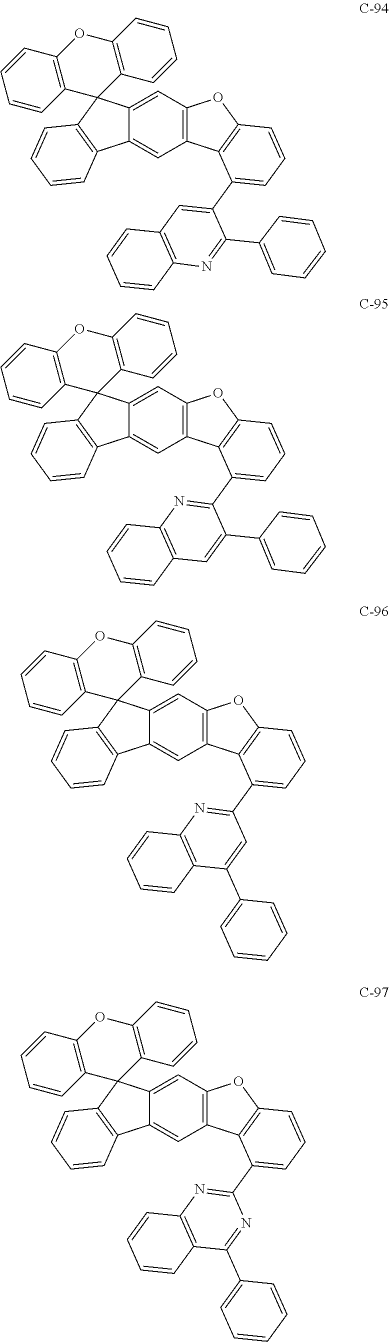

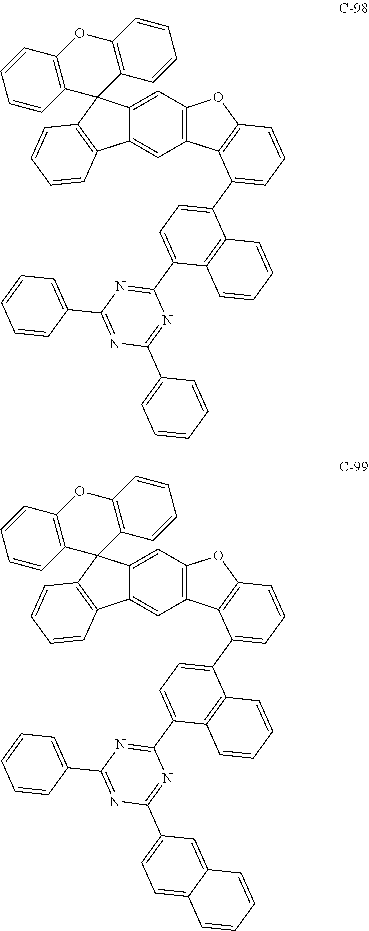

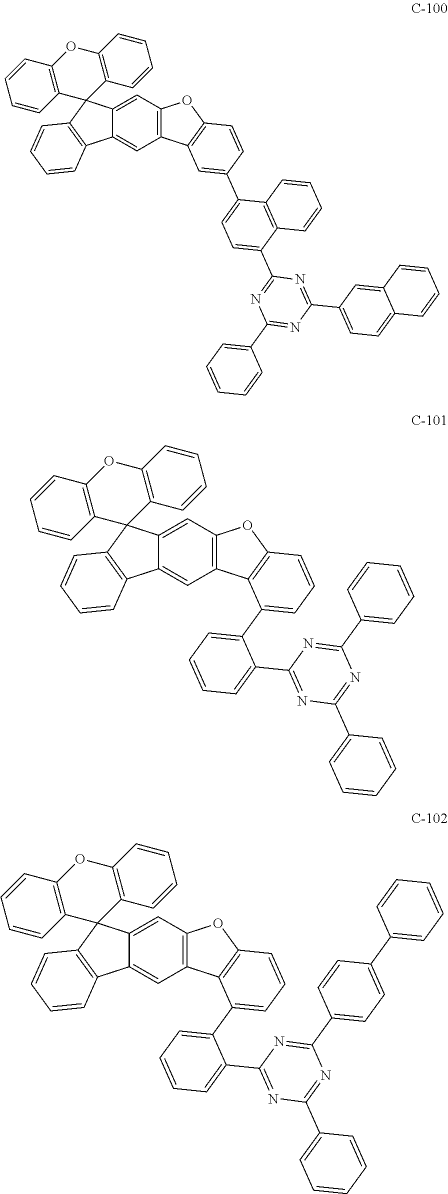 [SMILES: c1ccc(-c2nc(-c3ccc4ccccc4c3)nc(-c3ccc(-c4ccc5oc6cc7c(cc6c5c4)-c4ccccc4C74c5ccccc5Oc5ccccc54)c4ccccc34)n2)cc1 ; c1ccc(-c2nc(-c3ccccc3)nc(-c3ccccc3-c3cccc4oc5cc6c(cc5c34)-c3ccccc3C63c4ccccc4Oc4ccccc43)n2)cc1 ; c1ccc(-c2ccc(-c3nc(-c4ccccc4)nc(-c4ccccc4-c4cccc5oc6cc7c(cc6c45)-c4ccccc4C74c5ccccc5Oc5ccccc54)n3)cc2)cc1]

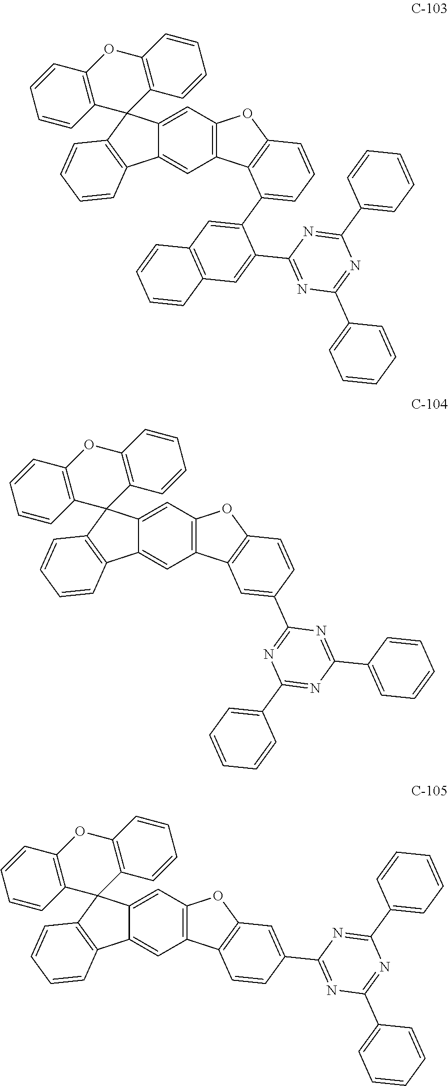 [SMILES: c1ccc(-c2nc(-c3ccccc3)nc(-c3cc4ccccc4cc3-c3cccc4oc5cc6c(cc5c34)-c3ccccc3C63c4ccccc4Oc4ccccc43)n2)cc1 ; c1ccc(-c2nc(-c3ccccc3)nc(-c3ccc4oc5cc6c(cc5c4c3)-c3ccccc3C63c4ccccc4Oc4ccccc43)n2)cc1 ; c1ccc(-c2nc(-c3ccccc3)nc(-c3ccc4c(c3)oc3cc5c(cc34)-c3ccccc3C53c4ccccc4Oc4ccccc43)n2)cc1]

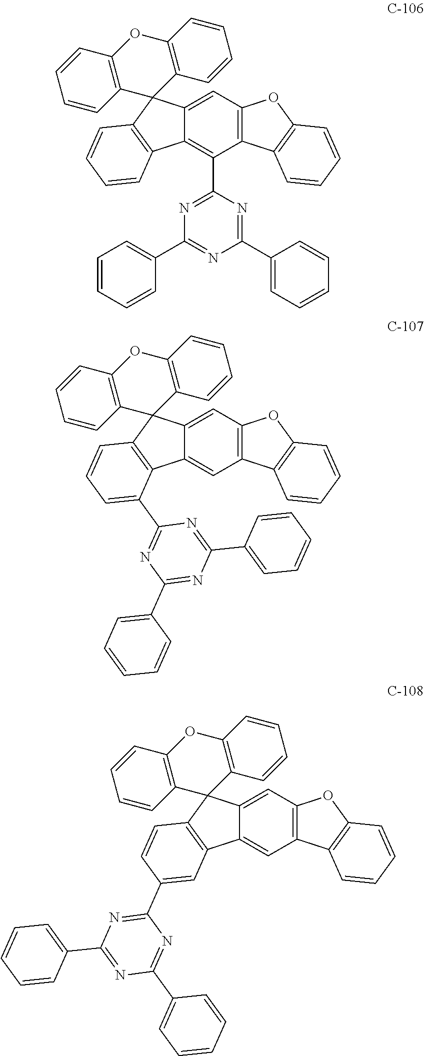

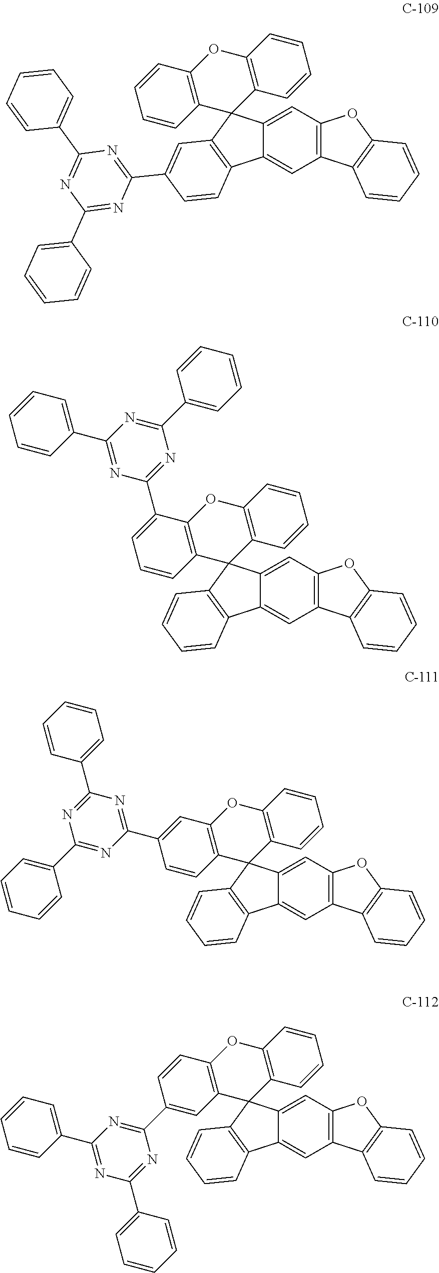 [SMILES: c1ccc(-c2nc(-c3ccccc3)nc(-c3ccc4c(c3)C3(c5ccccc5Oc5ccccc53)c3cc5oc6ccccc6c5cc3-4)n2)cc1 ; c1ccc(-c2nc(-c3ccccc3)nc(-c3cccc4c3Oc3ccccc3C43c4ccccc4-c4cc5c(cc43)oc3ccccc35)n2)cc1 ; c1ccc(-c2nc(-c3ccccc3)nc(-c3ccc4c(c3)Oc3ccccc3C43c4ccccc4-c4cc5c(cc43)oc3ccccc35)n2)cc1 ; c1ccc(-c2nc(-c3ccccc3)nc(-c3ccc4c(c3)C3(c5ccccc5O4)c4ccccc4-c4cc5c(cc43)oc3ccccc35)n2)cc1]

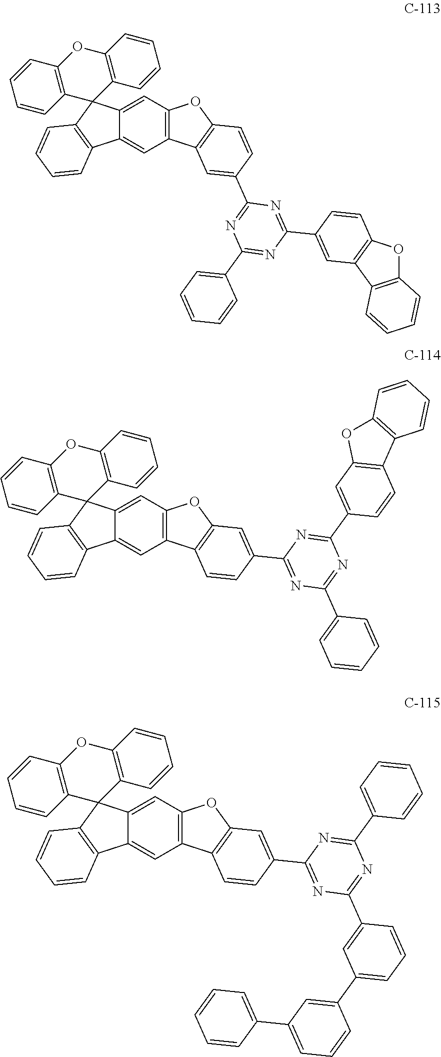 [SMILES: c1ccc(-c2nc(-c3ccc4oc5ccccc5c4c3)nc(-c3ccc4oc5cc6c(cc5c4c3)-c3ccccc3C63c4ccccc4Oc4ccccc43)n2)cc1 ; c1ccc(-c2nc(-c3ccc4c(c3)oc3ccccc34)nc(-c3ccc4c(c3)oc3cc5c(cc34)-c3ccccc3C53c4ccccc4Oc4ccccc43)n2)cc1 ; c1ccc(-c2cccc(-c3cccc(-c4nc(-c5ccccc5)nc(-c5ccc6c(c5)oc5cc7c(cc56)-c5ccccc5C75c6ccccc6Oc6ccccc65)n4)c3)c2)cc1]

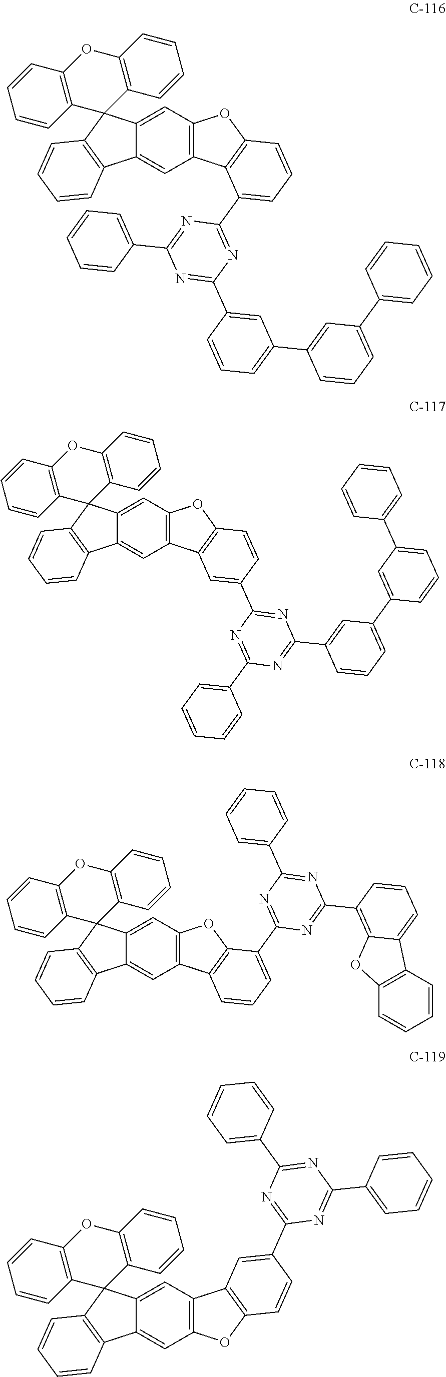 [SMILES: c1ccc(-c2cccc(-c3cccc(-c4nc(-c5ccccc5)nc(-c5cccc6oc7cc8c(cc7c56)-c5ccccc5C85c6ccccc6Oc6ccccc65)n4)c3)c2)cc1 ; c1ccc(-c2cccc(-c3cccc(-c4nc(-c5ccccc5)nc(-c5ccc6oc7cc8c(cc7c6c5)-c5ccccc5C85c6ccccc6Oc6ccccc65)n4)c3)c2)cc1 ; c1ccc(-c2nc(-c3cccc4c3oc3ccccc34)nc(-c3cccc4c3oc3cc5c(cc34)-c3ccccc3C53c4ccccc4Oc4ccccc43)n2)cc1 ; c1ccc(-c2nc(-c3ccccc3)nc(-c3ccc4oc5cc6c(cc5c4c3)C3(c4ccccc4Oc4ccccc43)c3ccccc3-6)n2)cc1]

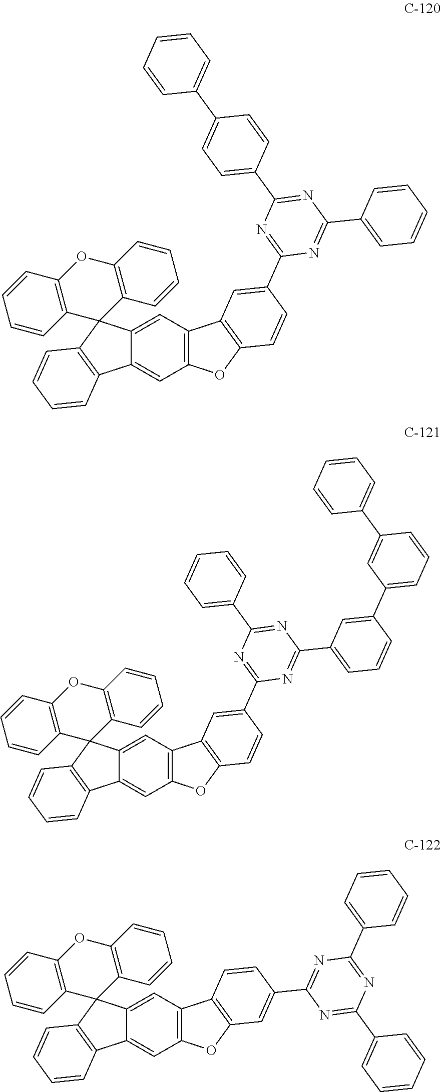 [SMILES: c1ccc(-c2ccc(-c3nc(-c4ccccc4)nc(-c4ccc5oc6cc7c(cc6c5c4)C4(c5ccccc5Oc5ccccc54)c4ccccc4-7)n3)cc2)cc1 ; c1ccc(-c2cccc(-c3cccc(-c4nc(-c5ccccc5)nc(-c5ccc6oc7cc8c(cc7c6c5)C5(c6ccccc6Oc6ccccc65)c5ccccc5-8)n4)c3)c2)cc1 ; c1ccc(-c2nc(-c3ccccc3)nc(-c3ccc4c(c3)oc3cc5c(cc34)C3(c4ccccc4Oc4ccccc43)c3ccccc3-5)n2)cc1]

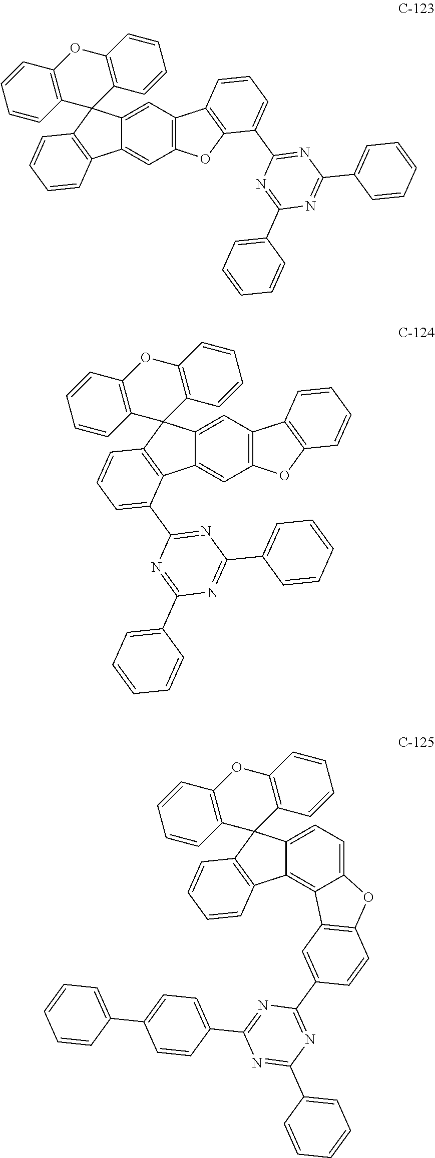 [SMILES: c1ccc(-c2nc(-c3ccccc3)nc(-c3cccc4c3oc3cc5c(cc34)C3(c4ccccc4Oc4ccccc43)c3ccccc3-5)n2)cc1 ; c1ccc(-c2nc(-c3ccccc3)nc(-c3cccc4c3-c3cc5oc6ccccc6c5cc3C43c4ccccc4Oc4ccccc43)n2)cc1 ; c1ccc(-c2ccc(-c3nc(-c4ccccc4)nc(-c4ccc5oc6ccc7c(c6c5c4)-c4ccccc4C74c5ccccc5Oc5ccccc54)n3)cc2)cc1]

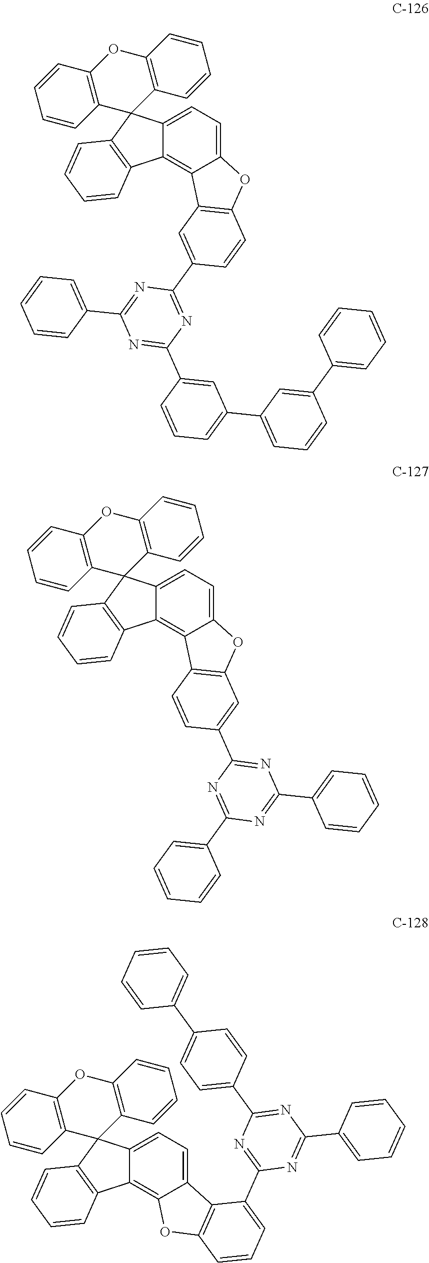 [SMILES: c1ccc(-c2cccc(-c3cccc(-c4nc(-c5ccccc5)nc(-c5ccc6oc7ccc8c(c7c6c5)-c5ccccc5C85c6ccccc6Oc6ccccc65)n4)c3)c2)cc1 ; c1ccc(-c2nc(-c3ccccc3)nc(-c3ccc4c(c3)oc3ccc5c(c34)-c3ccccc3C53c4ccccc4Oc4ccccc43)n2)cc1 ; c1ccc(-c2ccc(-c3nc(-c4ccccc4)nc(-c4cccc5oc6c7c(ccc6c45)C4(c5ccccc5Oc5ccccc54)c4ccccc4-7)n3)cc2)cc1]

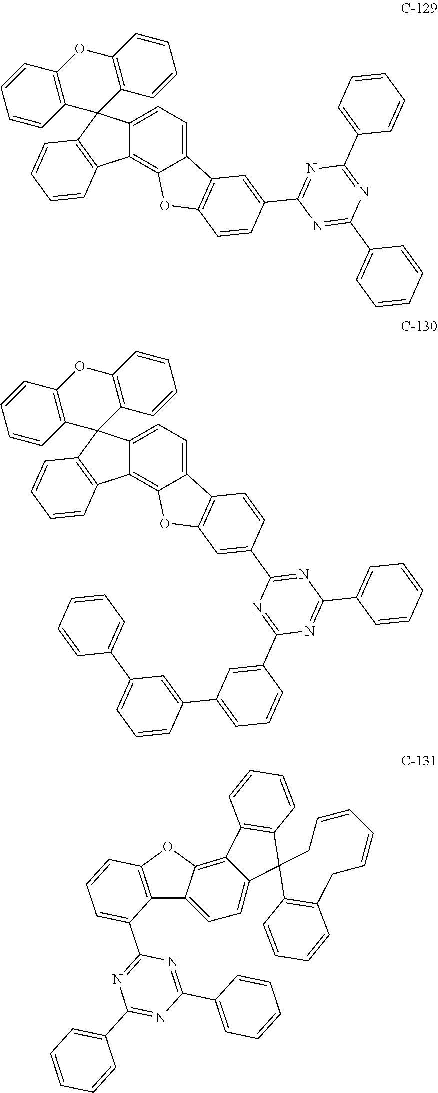

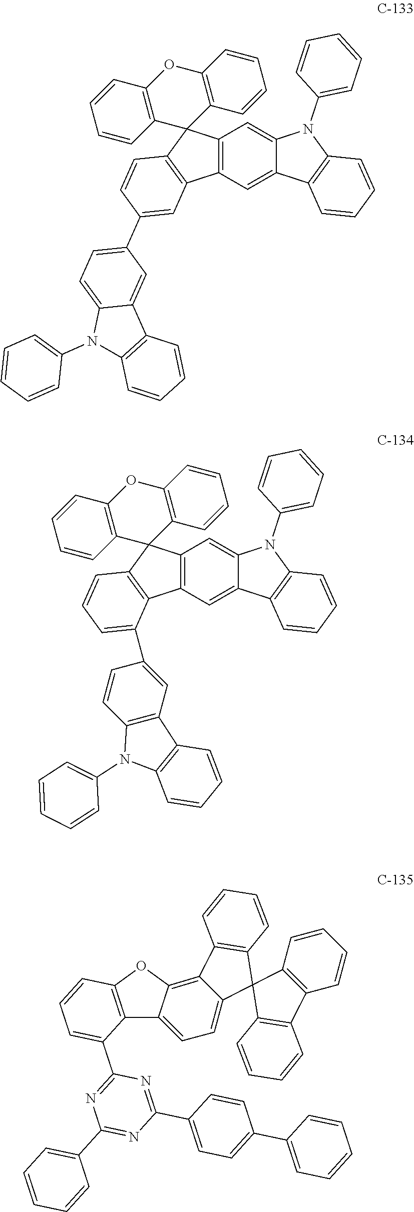 [SMILES: c1ccc(-n2c3ccccc3c3cc(-c4ccc5c(c4)-c4cc6c7ccccc7n(-c7ccccc7)c6cc4C54c5ccccc5Oc5ccccc54)ccc32)cc1 ; c1ccc(-n2c3ccccc3c3cc(-c4cccc5c4-c4cc6c7ccccc7n(-c7ccccc7)c6cc4C54c5ccccc5Oc5ccccc54)ccc32)cc1 ; c1ccc(-c2ccc(-c3nc(-c4ccccc4)nc(-c4cccc5oc6c7c(ccc6c45)C4(c5ccccc5-c5ccccc54)c4ccccc4-7)n3)cc2)cc1]

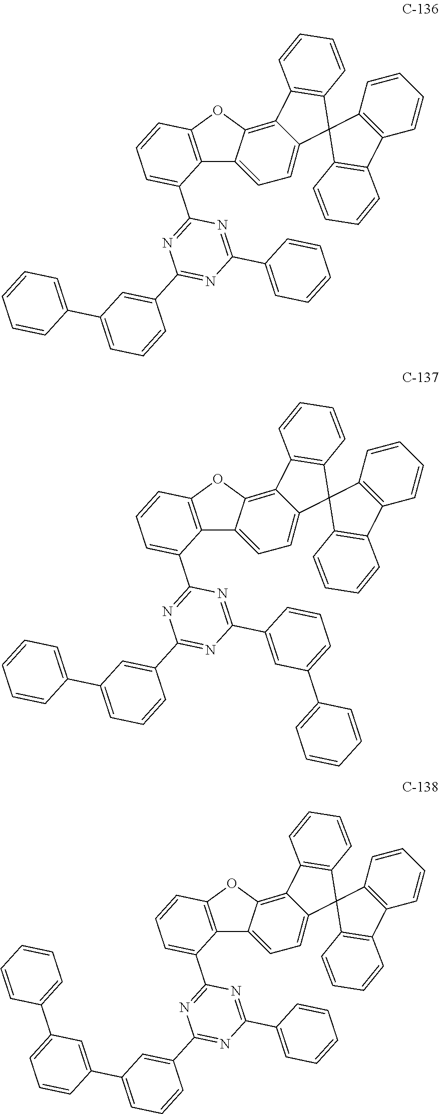 [SMILES: c1ccc(-c2cccc(-c3nc(-c4ccccc4)nc(-c4cccc5oc6c7c(ccc6c45)C4(c5ccccc5-c5ccccc54)c4ccccc4-7)n3)c2)cc1 ; c1ccc(-c2cccc(-c3nc(-c4cccc(-c5ccccc5)c4)nc(-c4cccc5oc6c7c(ccc6c45)C4(c5ccccc5-c5ccccc54)c4ccccc4-7)n3)c2)cc1 ; c1ccc(-c2cccc(-c3cccc(-c4nc(-c5ccccc5)nc(-c5cccc6oc7c8c(ccc7c56)C5(c6ccccc6-c6ccccc65)c5ccccc5-8)n4)c3)c2)cc1]

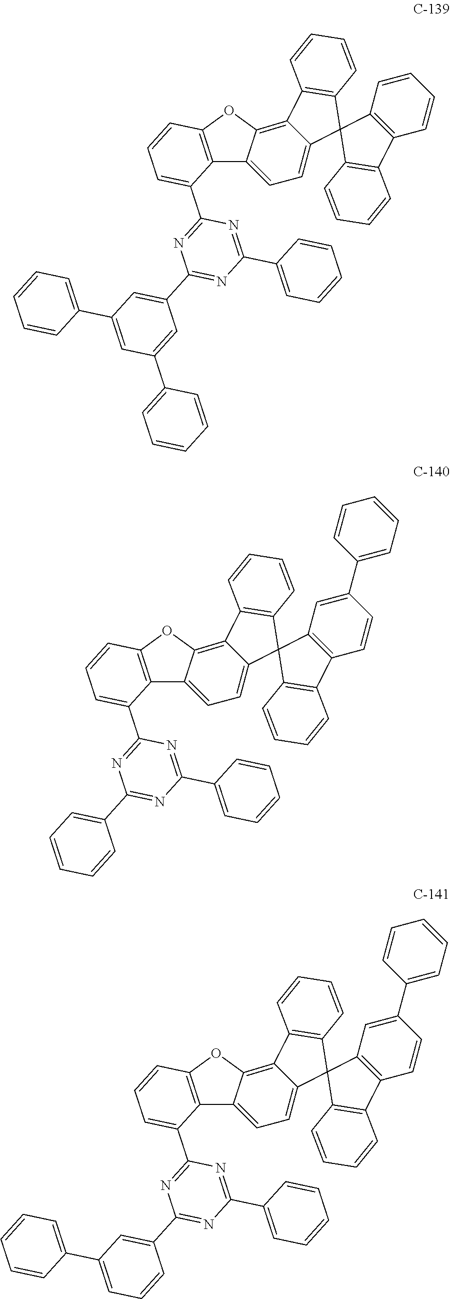 [SMILES: c1ccc(-c2cc(-c3ccccc3)cc(-c3nc(-c4ccccc4)nc(-c4cccc5oc6c7c(ccc6c45)C4(c5ccccc5-c5ccccc54)c4ccccc4-7)n3)c2)cc1 ; c1ccc(-c2ccc3c(c2)C2(c4ccccc4-3)c3ccccc3-c3c2ccc2c3oc3cccc(-c4nc(-c5ccccc5)nc(-c5ccccc5)n4)c32)cc1 ; c1ccc(-c2cccc(-c3nc(-c4ccccc4)nc(-c4cccc5oc6c7c(ccc6c45)C4(c5ccccc5-c5ccc(-c6ccccc6)cc54)c4ccccc4-7)n3)c2)cc1]

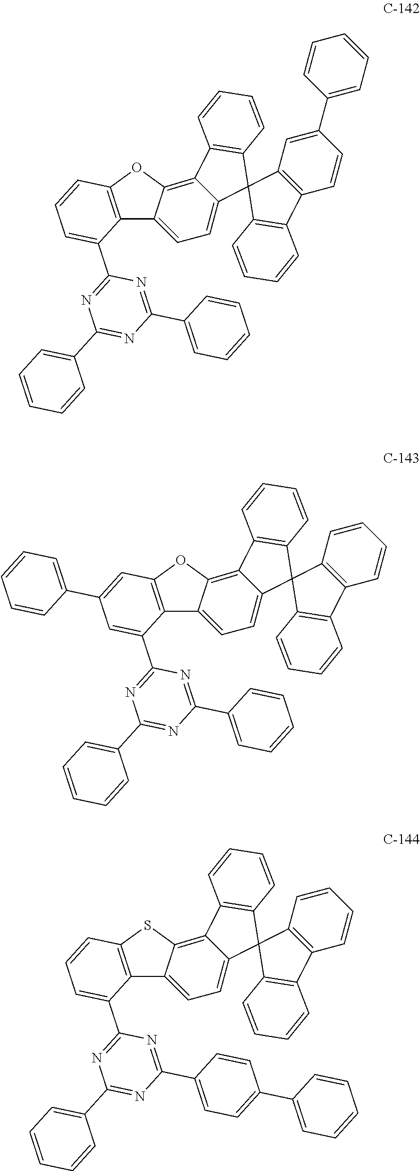 [SMILES: c1ccc(-c2ccc3c(c2)C2(c4ccccc4-3)c3ccccc3-c3c2ccc2c3oc3cccc(-c4nc(-c5ccccc5)nc(-c5ccccc5)n4)c32)cc1 ; c1ccc(-c2cc(-c3nc(-c4ccccc4)nc(-c4ccccc4)n3)c3c(c2)oc2c4c(ccc23)C2(c3ccccc3-c3ccccc32)c2ccccc2-4)cc1 ; c1ccc(-c2ccc(-c3nc(-c4ccccc4)nc(-c4cccc5sc6c7c(ccc6c45)C4(c5ccccc5-c5ccccc54)c4ccccc4-7)n3)cc2)cc1]

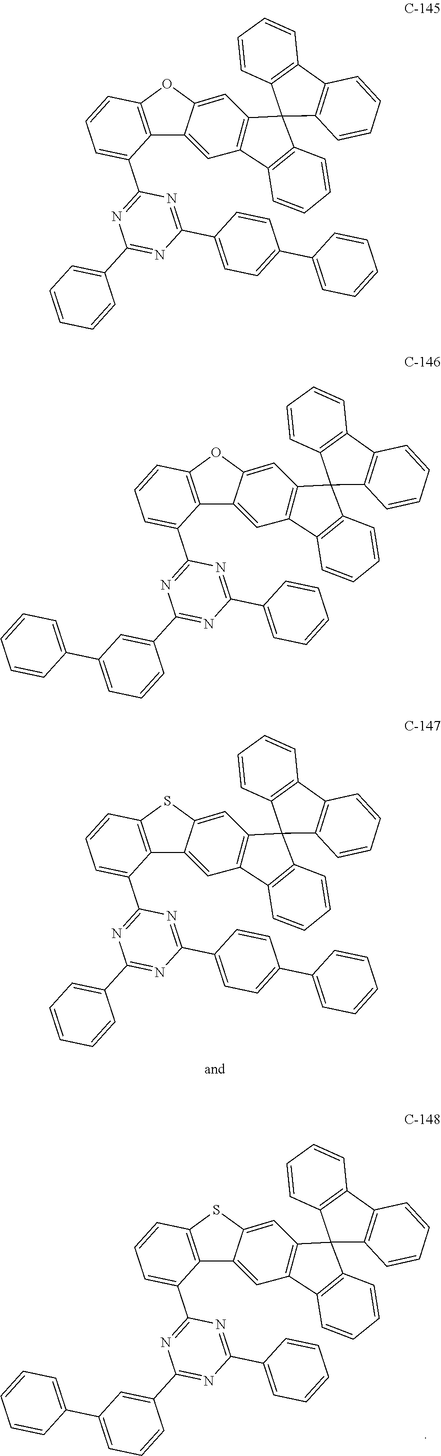 [SMILES: c1ccc(-c2ccc(-c3nc(-c4ccccc4)nc(-c4cccc5oc6cc7c(cc6c45)-c4ccccc4C74c5ccccc5-c5ccccc54)n3)cc2)cc1 ; c1ccc(-c2cccc(-c3nc(-c4ccccc4)nc(-c4cccc5oc6cc7c(cc6c45)-c4ccccc4C74c5ccccc5-c5ccccc54)n3)c2)cc1 ; c1ccc(-c2ccc(-c3nc(-c4ccccc4)nc(-c4cccc5sc6cc7c(cc6c45)-c4ccccc4C74c5ccccc5-c5ccccc54)n3)cc2)cc1 ; c1ccc(-c2cccc(-c3nc(-c4ccccc4)nc(-c4cccc5sc6cc7c(cc6c45)-c4ccccc4C74c5ccccc5-c5ccccc54)n3)c2)cc1]

11. The plurality of host materials according to claim 9, wherein the compound represented by formula 3 is selected from the following compounds:

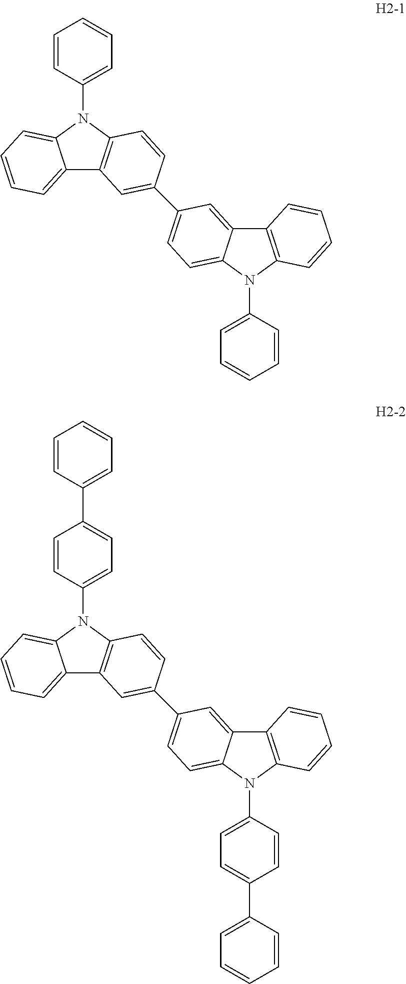 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

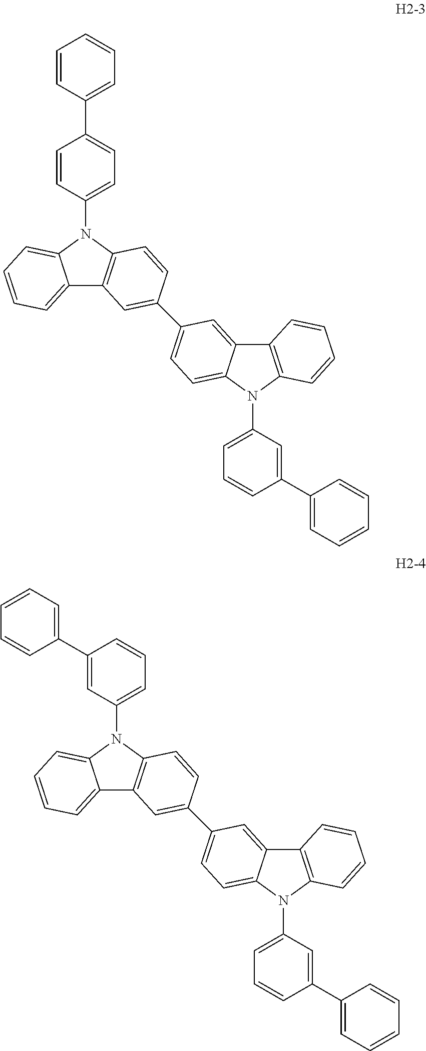 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

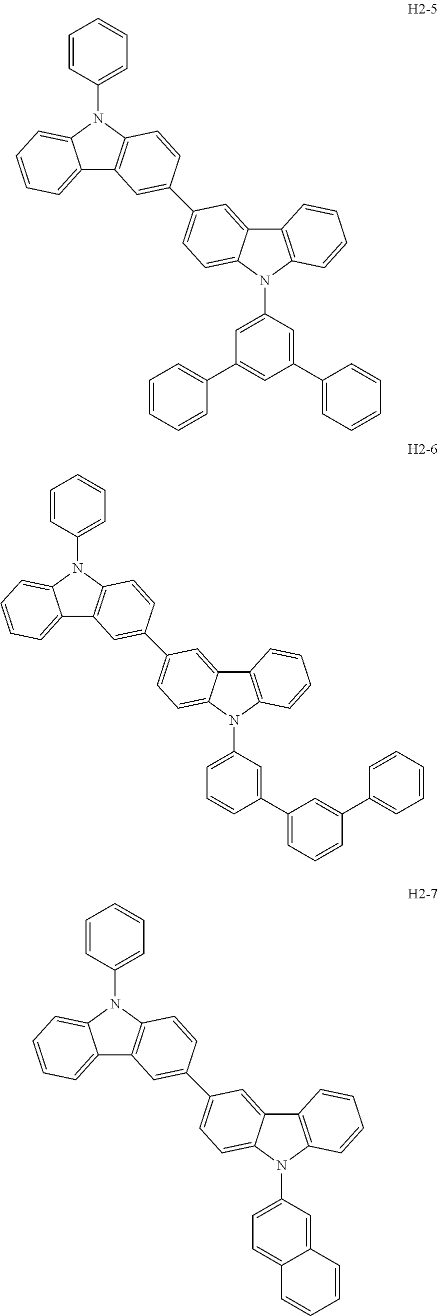 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2cccc(-c3cccc(-n4c5ccccc5c5cc(-c6ccc7c(c6)c6ccccc6n7-c6ccccc6)ccc54)c3)c2)cc1 ; c1ccc(-n2c3ccccc3c3cc(-c4ccc5c(c4)c4ccccc4n5-c4ccc5ccccc5c4)ccc32)cc1]

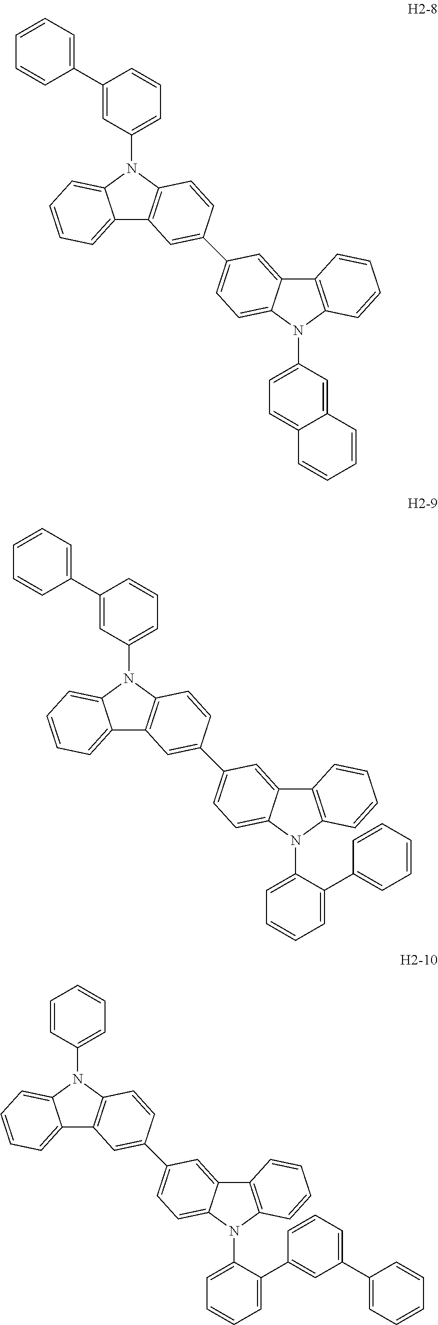 [SMILES: c1ccc(-c2cccc(-n3c4ccccc4c4cc(-c5ccc6c(c5)c5ccccc5n6-c5ccc6ccccc6c5)ccc43)c2)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2cccc(-c3ccccc3-n3c4ccccc4c4cc(-c5ccc6c(c5)c5ccccc5n6-c5ccccc5)ccc43)c2)cc1]

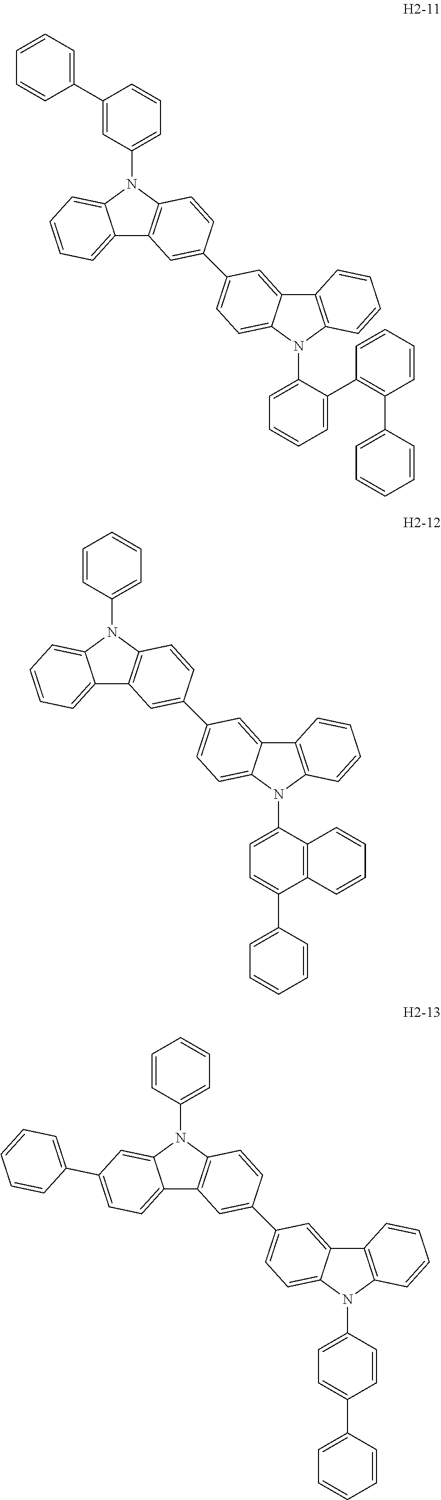 [SMILES: c1ccc(-c2cccc(-n3c4ccccc4c4cc(-c5ccc6c(c5)c5ccccc5n6-c5ccccc5-c5ccccc5-c5ccccc5)ccc43)c2)cc1 ; c1ccc(-c2ccc(-n3c4ccccc4c4cc(-c5ccc6c(c5)c5ccccc5n6-c5ccccc5)ccc43)c3ccccc23)cc1 ; c1ccc(-c2ccc(-n3c4ccccc4c4cc(-c5ccc6c(c5)c5ccc(-c7ccccc7)cc5n6-c5ccccc5)ccc43)cc2)cc1]

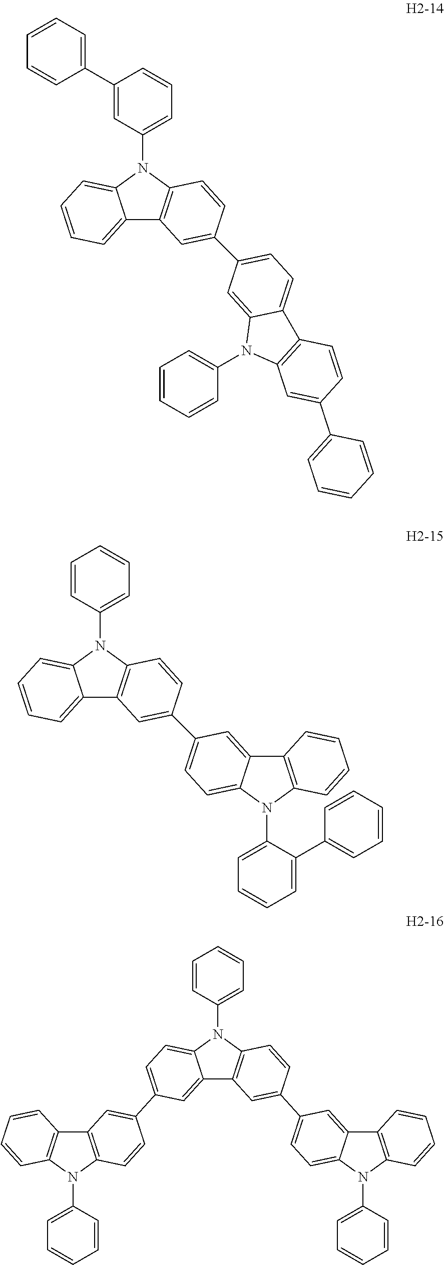 [SMILES: c1ccc(-c2cccc(-n3c4ccccc4c4cc(-c5ccc6c7ccc(-c8ccccc8)cc7n(-c7ccccc7)c6c5)ccc43)c2)cc1 ; c1ccc(-c2ccccc2-n2c3ccccc3c3cc(-c4ccc5c(c4)c4ccccc4n5-c4ccccc4)ccc32)cc1 ; c1ccc(-n2c3ccccc3c3cc(-c4ccc5c(c4)c4cc(-c6ccc7c(c6)c6ccccc6n7-c6ccccc6)ccc4n5-c4ccccc4)ccc32)cc1]

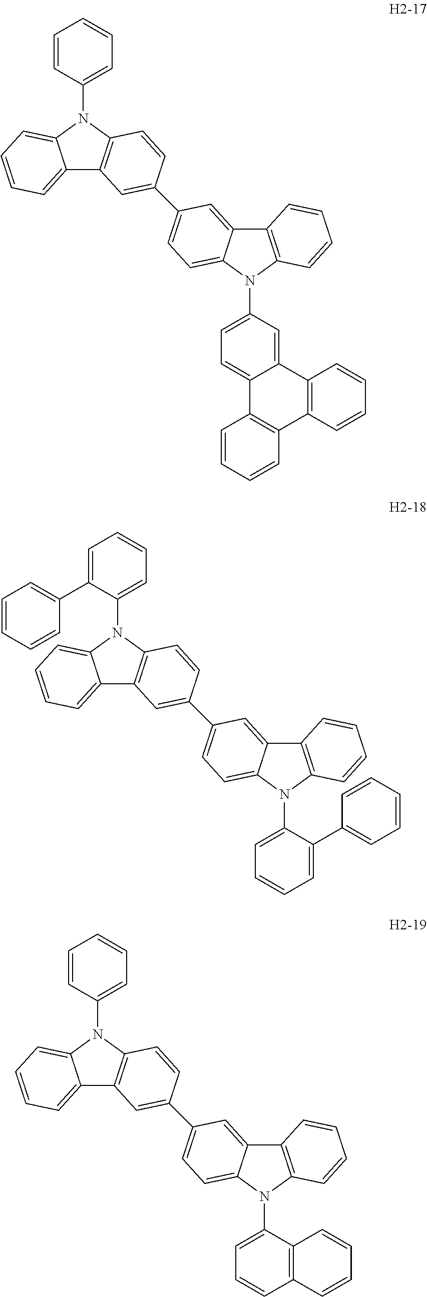 [SMILES: c1ccc(-n2c3ccccc3c3cc(-c4ccc5c(c4)c4ccccc4n5-c4ccc5c6ccccc6c6ccccc6c5c4)ccc32)cc1 ; c1ccc(-c2ccccc2-n2c3ccccc3c3cc(-c4ccc5c(c4)c4ccccc4n5-c4ccccc4-c4ccccc4)ccc32)cc1 ; c1ccc(-n2c3ccccc3c3cc(-c4ccc5c(c4)c4ccccc4n5-c4cccc5ccccc45)ccc32)cc1]

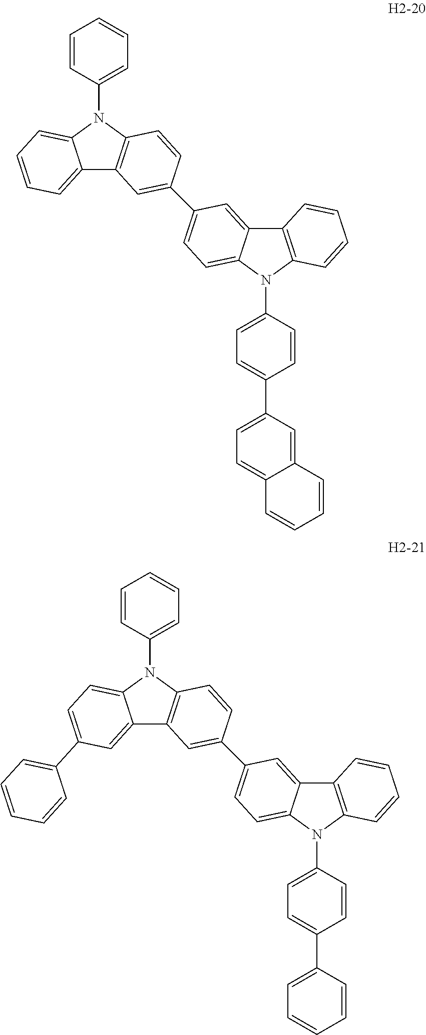 [SMILES: c1ccc(-n2c3ccccc3c3cc(-c4ccc5c(c4)c4ccccc4n5-c4ccc(-c5ccc6ccccc6c5)cc4)ccc32)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

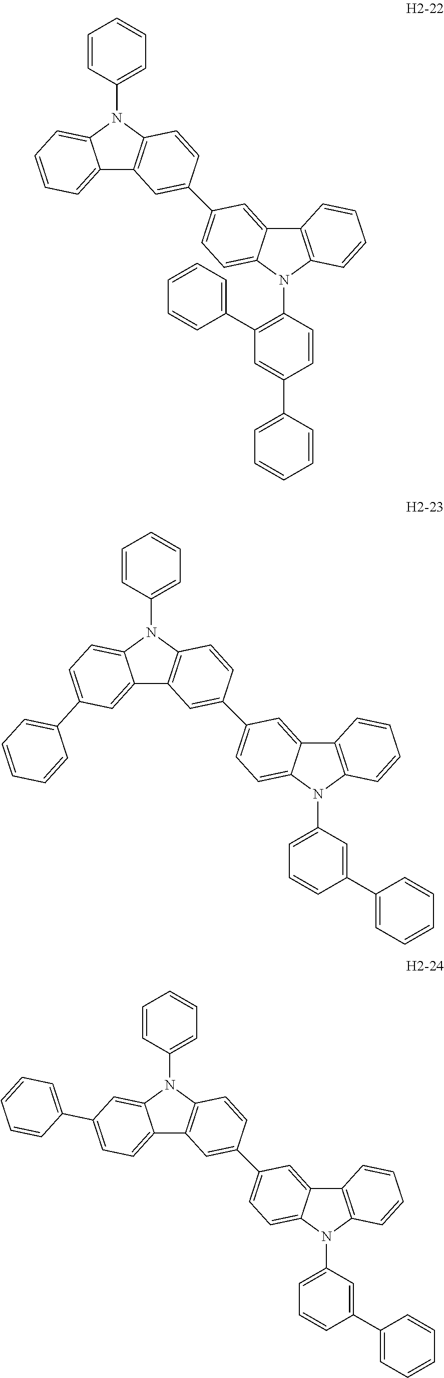 [SMILES: c1ccc(-c2ccc(-n3c4ccccc4c4cc(-c5ccc6c(c5)c5ccccc5n6-c5ccccc5)ccc43)c(-c3ccccc3)c2)cc1 ; c1ccc(-c2cccc(-n3c4ccccc4c4cc(-c5ccc6c(c5)c5cc(-c7ccccc7)ccc5n6-c5ccccc5)ccc43)c2)cc1 ; c1ccc(-c2cccc(-n3c4ccccc4c4cc(-c5ccc6c(c5)c5ccc(-c7ccccc7)cc5n6-c5ccccc5)ccc43)c2)cc1]

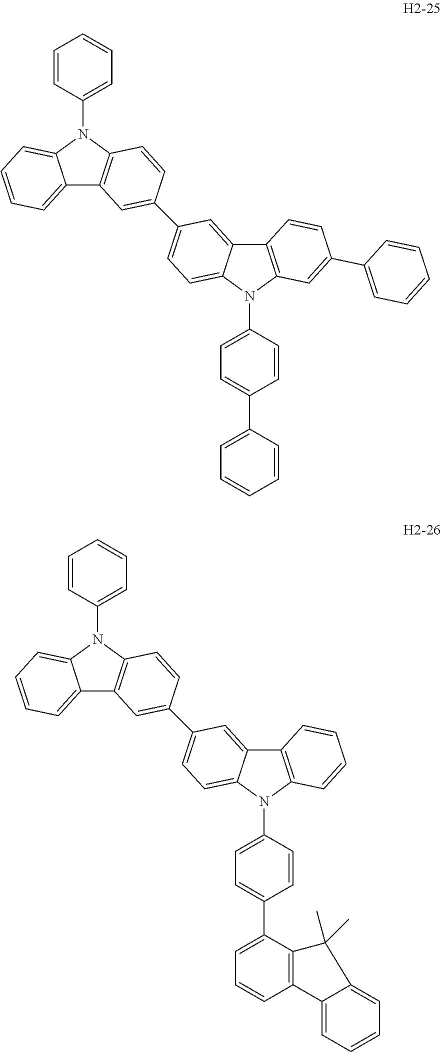 [SMILES: c1ccc(-c2ccc(-n3c4ccc(-c5ccc6c(c5)c5ccccc5n6-c5ccccc5)cc4c4ccc(-c5ccccc5)cc43)cc2)cc1 ; CC1(C)c2ccccc2-c2cccc(-c3ccc(-n4c5ccccc5c5cc(-c6ccc7c(c6)c6ccccc6n7-c6ccccc6)ccc54)cc3)c21]

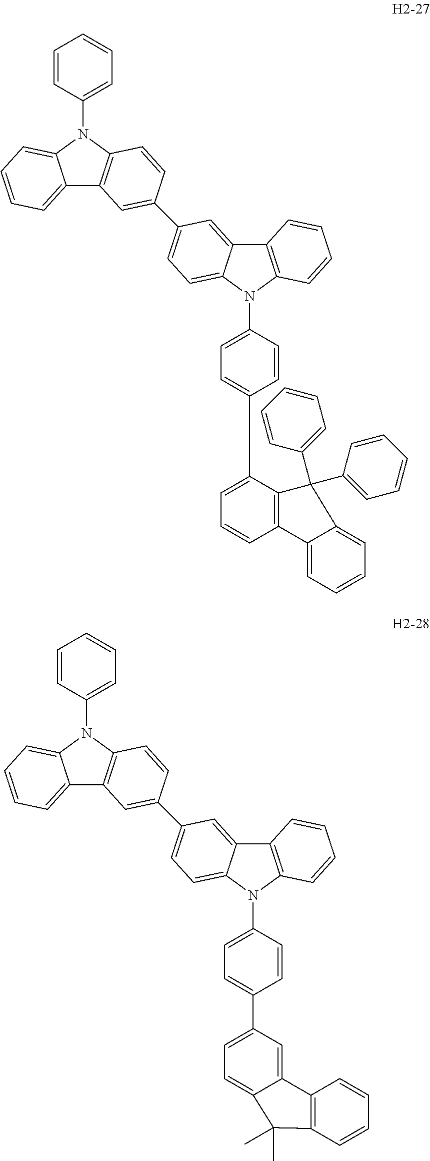 [SMILES: c1ccc(-n2c3ccccc3c3cc(-c4ccc5c(c4)c4ccccc4n5-c4ccc(-c5cccc6c5C(c5ccccc5)(c5ccccc5)c5ccccc5-6)cc4)ccc32)cc1 ; CC1(C)c2ccccc2-c2cc(-c3ccc(-n4c5ccccc5c5cc(-c6ccc7c(c6)c6ccccc6n7-c6ccccc6)ccc54)cc3)ccc21]

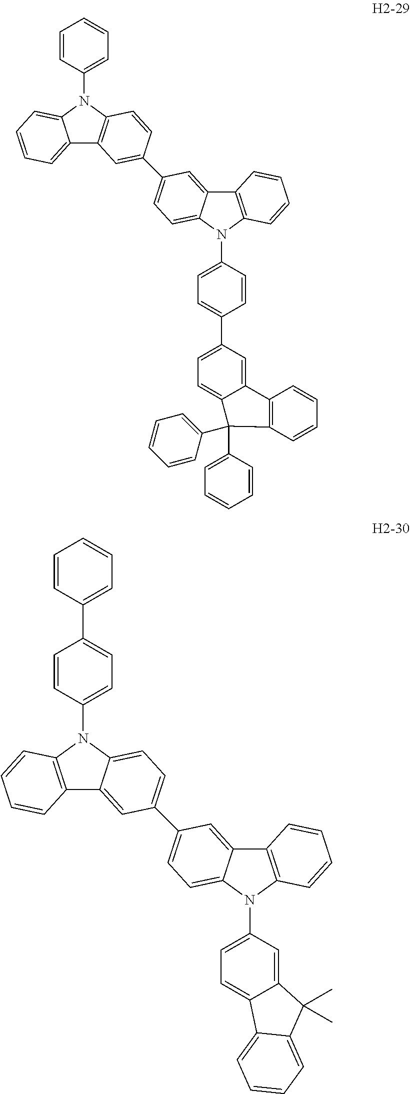 [SMILES: c1ccc(-n2c3ccccc3c3cc(-c4ccc5c(c4)c4ccccc4n5-c4ccc(-c5ccc6c(c5)-c5ccccc5C6(c5ccccc5)c5ccccc5)cc4)ccc32)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21]

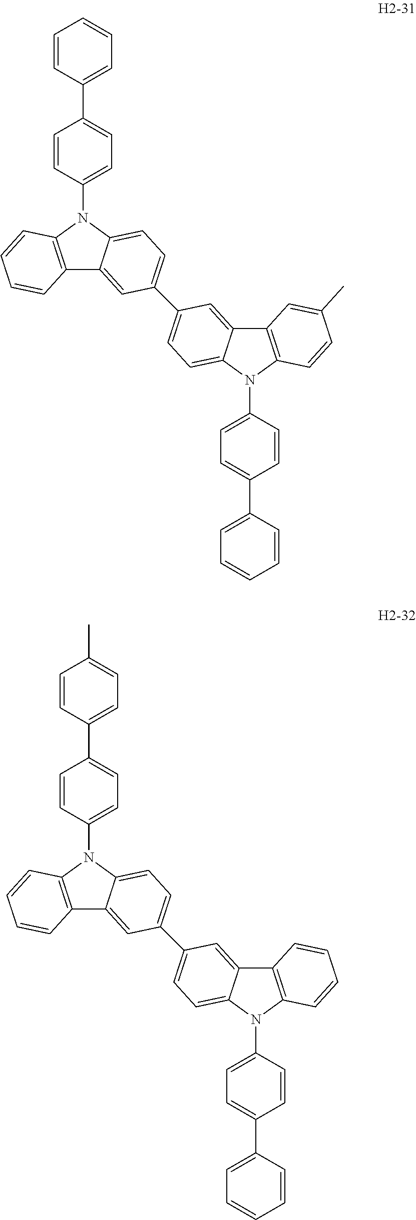 [SMILES: Cc1ccc2c(c1)c1cc(-c3ccc4c(c3)c3ccccc3n4-c3ccc(-c4ccccc4)cc3)ccc1n2-c1ccc(-c2ccccc2)cc1 ; Cc1ccc(-c2ccc(-n3c4ccccc4c4cc(-c5ccc6c(c5)c5ccccc5n6-c5ccc(-c6ccccc6)cc5)ccc43)cc2)cc1]

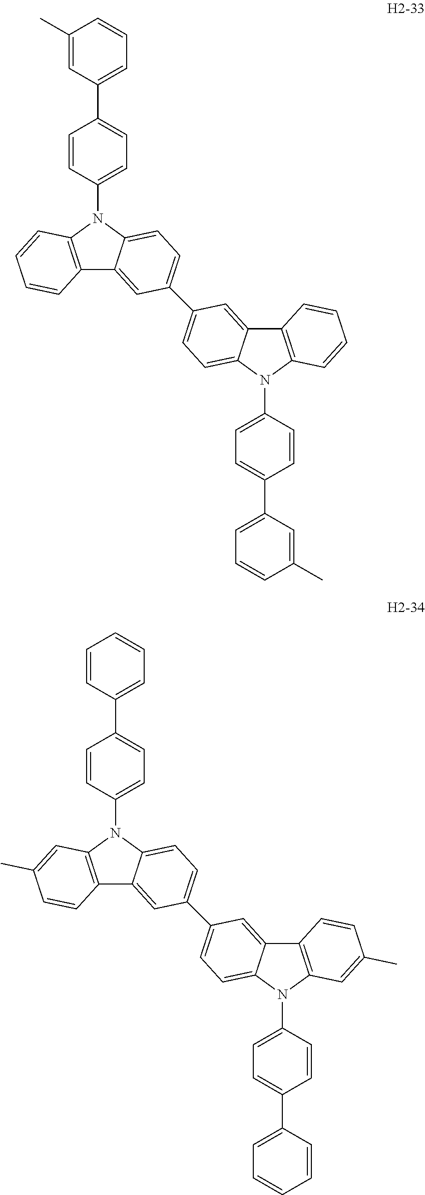

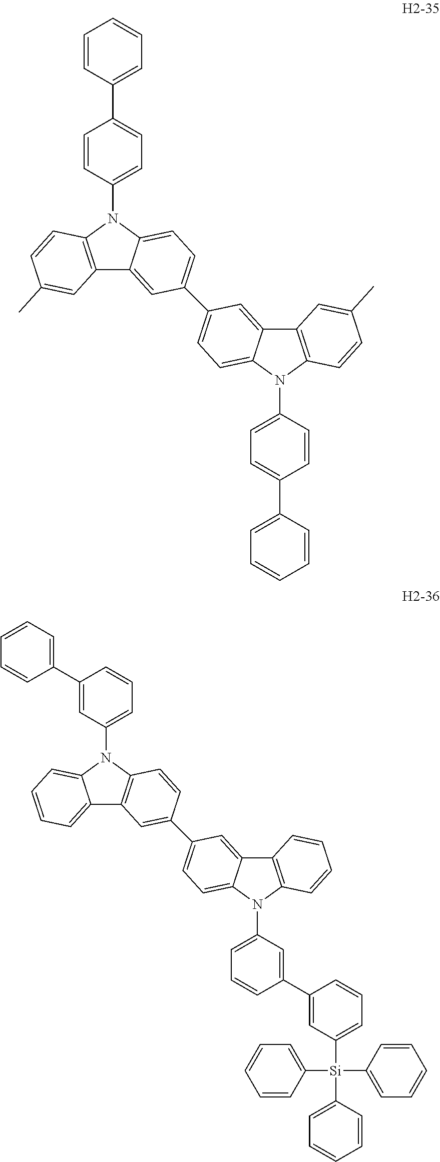

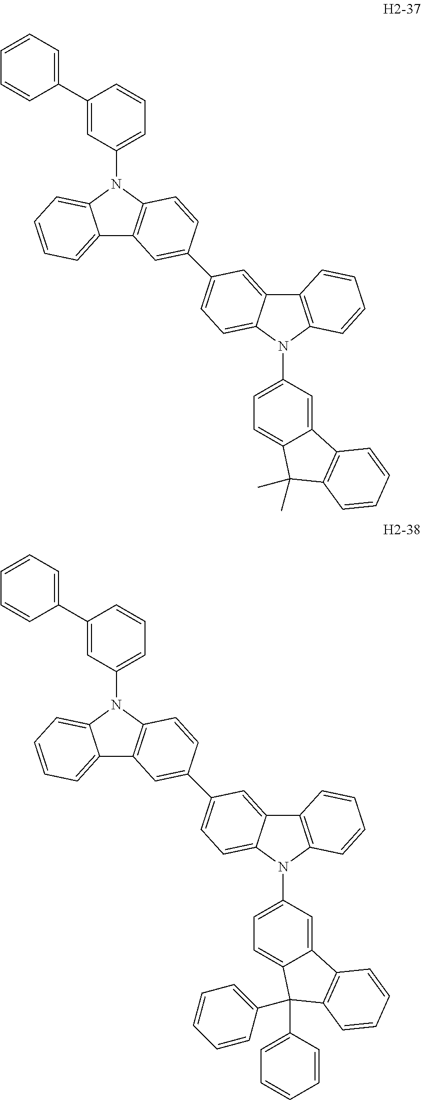 [SMILES: CC1(C)c2ccccc2-c2cc(-n3c4ccccc4c4cc(-c5ccc6c(c5)c5ccccc5n6-c5cccc(-c6ccccc6)c5)ccc43)ccc21 ; c1ccc(-c2cccc(-n3c4ccccc4c4cc(-c5ccc6c(c5)c5ccccc5n6-c5ccc6c(c5)-c5ccccc5C6(c5ccccc5)c5ccccc5)ccc43)c2)cc1]

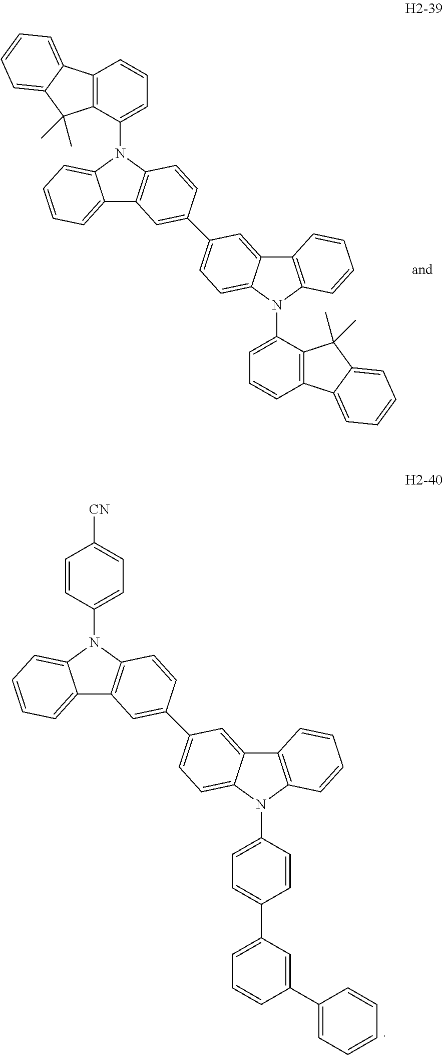 [SMILES: CC1(C)c2ccccc2-c2cccc(-n3c4ccccc4c4cc(-c5ccc6c(c5)c5ccccc5n6-c5cccc6c5C(C)(C)c5ccccc5-6)ccc43)c21 ; N#Cc1ccc(-n2c3ccccc3c3cc(-c4ccc5c(c4)c4ccccc4n5-c4ccc(-c5cccc(-c6ccccc6)c5)cc4)ccc32)cc1]

12. An organic electroluminescent device comprising the organic electroluminescent compound according to claim 8.

13. An organic electroluminescent device comprising an anode, a cathode, and at least one light-emitting layer between the anode and the cathode, wherein at least one layer of the light-emitting layers comprises the plurality of host materials according to claim 9.
